SEQUENCE LISTING (253 sequences)

SEQ ID NO: 1 - protein, 529 aa, unidentified
  REGION 1..529: note = Description of Unknown: Cas14 sequence
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 2 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 3 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 5 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKERP

SEQ ID NO: 6 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKERP

SEQ ID NO: 7 - DNA, 228 bp, unidentified
  misc_feature 1..228: note = Description of Unknown: sgRNA sequence
  variation 206..228: note = n = a, c, t, g, unknown or other
  source 1..228: mol_type = genomic DNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacnnnnnnnnnnnnnnnnnnnnnnn

SEQ ID NO: 8 - DNA, 228 bp, synthetic construct
  misc_feature 1..228: note = Description of sequence: Synthetic polynucleotide
  variation 206..228: note = n = a, c, t, g, unknown or other
  source 1..228: mol_type = other DNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattgttcctctccaattctgcacaagaaagttgcagaacccgaatagacgaatgaaggaatgcaacnnnnnnnnnnnnnnnnnnnnnnn

SEQ ID NO: 9 - DNA, 182 bp, synthetic construct
  misc_feature 1..182: note = Description of sequence: Synthetic polynucleotide
  variation 161..182: note = n = a, c, t, g, unknown or other
  source 1..182: mol_type = other DNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnnnnn

SEQ ID NO: 10 - DNA, 181 bp, synthetic construct
  misc_feature 1..181: note = Description of sequence: Synthetic polynucleotide
  variation 160..181: note = n = a, c, t, g, unknown or other
  source 1..181: mol_type = other DNA
ggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnnnnn

SEQ ID NO: 11 - protein, 529 aa, synthetic construct
  REGION 1..529: note = Description of sequence: Modified CAS protein
MAKNTITKTLRLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 12 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCRARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 13 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKRAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 14 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 15 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAARLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 16 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFRNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 17 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKRAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 18 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVRQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 19 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFRQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 20 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIRVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 21 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVRPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 22 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDRGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 23 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 24 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLNSMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 25 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLRQYGIEIRKVAPNNTSKTCSKCGHLNNYFNFEYRKKNKFPHFKCEKCNFKENADYNAALNISNPKLKSTKEEP

SEQ ID NO: 26 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIRIRKVAPNNTSKTCSKCGHLNNYFNFEYRKKNKFPHFKCEKCNFKENADYNAALNISNPKLKSTKEEP

SEQ ID NO: 27 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKREKCNFKENADYNAALNISNPKLKSTKEEP

SEQ ID NO: 28 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFRREKCNFKENADYNAALNISNPKLKSTKEEP

SEQ ID NO: 29 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCRKCNFKENADYNAALNISNPKLKSTKEEP

SEQ ID NO: 30 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCRFKENADYNAALNISNPKLKSTKEEP

SEQ ID NO: 31 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNADYNAALNISNPKLKSTKEEP

SEQ ID NO: 32 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKEDADYNAALNISNPKLKSTKEEP

SEQ ID NO: 33 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADANAALNISNPKLKSTKEEP

SEQ ID NO: 34 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAGLNISNPKLKSTKEEP

SEQ ID NO: 35 - protein, 528 aa, synthetic construct
  REGION 1..528: note = Description of sequence: Modified CAS protein
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALRISNPKLKSTKEEP

SEQ ID NO: 36 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISRPKLKSTKEEP

SEQ ID NO: 37 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKREP

SEQ ID NO: 38 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKERP

SEQ ID NO: 39 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAARLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 40 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFRNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 41 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFRQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 42 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 43 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLNSMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 44 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNADYNAALNISNPKLKSTKEEP

SEQ ID NO: 45 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALRISNPKLKSTKEEP

SEQ ID NO: 46 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISRPKLKSTKEEP

SEQ ID NO: 47 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKREP

SEQ ID NO: 48 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKERP

SEQ ID NO: 49 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 50 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFRNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 51 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFRQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 52 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLNSMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 53 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNADYNAALNISNPKLKSTKEEP

SEQ ID NO: 54 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALRISNPKLKSTKEEP

SEQ ID NO: 55 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISRPKLKSTKEEP

SEQ ID NO: 56 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKREP

SEQ ID NO: 57 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKERP

SEQ ID NO: 58 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAASLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 59 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 60 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 61 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAALLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 62 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAASLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 63 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAATLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 64 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAWLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 65 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAFLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 66 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAACLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 67 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAKLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 68 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAGLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 69 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAILFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 70 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAPLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 71 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAHLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 72 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 73 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAVLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 74 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAQLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 75 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAMLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 76 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAADLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 77 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAANLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 78 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAYLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 79 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIDVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKREP

SEQ ID NO: 80 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 81 - protein, 529 aa, synthetic construct
  REGION 1..529: note = Description of sequence: Modified CAS protein
MAKNTITKTLRLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 82 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCRARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 83 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKRAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 84 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 85 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 86 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAARLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 87 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFRNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 88 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKRAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 89 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVRQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 90 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFRQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 91 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIRVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 92 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVRPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 93 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIARGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 94 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 95 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLNSMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 96 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLRQYGIEIRKVAPNNTSKTCSKCGHLNNYFNFEYRKKNKFPHFKCEKCNFKENAAYNAALNISNPKLKSTKEEP

SEQ ID NO: 97 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIRIRKVAPNNTSKTCSKCGHLNNYFNFEYRKKNKFPHFKCEKCNFKENAAYNAALNISNPKLKSTKEEP

SEQ ID NO: 98 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKREKCNFKENAAYNAALNISNPKLKSTKEEP

SEQ ID NO: 99 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFRREKCNFKENAAYNAALNISNPKLKSTKEEP

SEQ ID NO: 100 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCRKCNFKENAAYNAALNISNPKLKSTKEEP

SEQ ID NO: 101 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCRFKENAAYNAALNISNPKLKSTKEEP

SEQ ID NO: 102 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNAAYNAALNISNPKLKSTKEEP

SEQ ID NO: 103 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKEDAAYNAALNISNPKLKSTKEEP

SEQ ID NO: 104 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAANAALNISNPKLKSTKEEP

SEQ ID NO: 105 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAGLNISNPKLKSTKEEP

SEQ ID NO: 106 - protein, 528 aa, synthetic construct
  REGION 1..528: note = Description of sequence: Modified CAS protein
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALRISNPKLKSTKEEP

SEQ ID NO: 107 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISRPKLKSTKEEP

SEQ ID NO: 108 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKREP

SEQ ID NO: 109 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 110 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 111 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAARLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 112 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFRNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 113 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFRQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 114 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 115 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLNSMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 116 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNAAYNAALNISNPKLKSTKEEP

SEQ ID NO: 117 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALRISNPKLKSTKEEP

SEQ ID NO: 118 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISRPKLKSTKEEP

SEQ ID NO: 119 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKREP

SEQ ID NO: 120 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 121 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAARLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 122 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFRNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 123 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFRQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 124 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 125 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLNSMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 126 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKRNAAYNAALNISNPKLKSTKEEP

SEQ ID NO: 127 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALRISNPKLKSTKEEP

SEQ ID NO: 128 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISRPKLKSTKEEP

SEQ ID NO: 129 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKREP

SEQ ID NO: 130 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 131 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAASLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 132 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAGLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 133 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 134 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAALLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 135 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAASLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 136 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAATLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 137 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAWLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 138 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAFLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 139 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAACLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 140 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAKLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 141 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAGLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 142 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAILFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 143 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAPLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 144 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAHLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 145 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 146 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAVLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 147 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAQLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 148 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAMLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 149 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAADLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 150 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAANLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 151 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAYLFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 152 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKREP

SEQ ID NO: 153 - protein, 529 aa, synthetic construct
  REGION 1..529: note = Description of sequence: Modified CAS protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAALFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 154 - protein, 1614 aa, synthetic construct
  REGION 1..1614: note = Description of sequence: Modified CAS protein
PKKKRKVGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSYPYDVPDYASLGSGDGIGSGSNGSSLMDAKSLTAWSRTLVTFKDVFVDFTREEWKLLDTAQQIVYRNVMLENYKNLVSLGYQLTKPDVILRLEKGEEPHMGGGSGEDPAAKRVKLDMGSGATNFSLLKQAGDVEENPGPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 155 - protein, 1652 aa, synthetic construct
  REGION 1..1652: note = Description of sequence: Modified CAS protein
PKKKRKVGSGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSYPYDVPDYASLGSGDGIGSGSNGSSLMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKEHMGGGSGEDPAAKRVKLDMGSGATNFSLLKQAGDVEENPGPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 156 - protein, 895 aa, synthetic construct
  REGION 1..895: note = Description of sequence: Modified CAS protein
PKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEPAYPYDVPDYASLGSGDGIGSGSNGSSLMDAKSLTAWSRTLVTFKDVFVDFTREEWKLLDTAQQIVYRNVMLENYKNLVSLGYQLTKPDVILRLEKGEEPHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 157 - protein, 933 aa, synthetic construct
  REGION 1..933: note = Description of sequence: Modified CAS protein
PKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEPAYPYDVPDYASLGSGDGIGSGSNGSSLMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKEHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 158 - protein, 895 aa, synthetic construct
  REGION 1..895: note = Description of sequence: Modified CAS protein
PKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEPAYPYDVPDYASLGSGDGIGSGSNGSSLMDAKSLTAWSRTLVTFKDVFVDFTREEWKLLDTAQQIVYRNVMLENYKNLVSLGYQLTKPDVILRLEKGEEPHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 159 - protein, 933 aa, synthetic construct
  REGION 1..933: note = Description of sequence: Modified CAS protein
PKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEPAYPYDVPDYASLGSGDGIGSGSNGSSLMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKEHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 160 - protein, 895 aa, synthetic construct
  REGION 1..895: note = Description of sequence: Modified CAS protein
PKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEPAYPYDVPDYASLGSGDGIGSGSNGSSLMDAKSLTAWSRTLVTFKDVFVDFTREEWKLLDTAQQIVYRNVMLENYKNLVSLGYQLTKPDVILRLEKGEEPHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 161 - protein, 933 aa, synthetic construct
  REGION 1..933: note = Description of sequence: Modified CAS protein
PKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEPAYPYDVPDYASLGSGDGIGSGSNGSSLMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKEHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 162 - DNA, 23 bp, synthetic construct
  misc_feature 1..23: note = Description of sequence: Synthetic oligonucleotide
  source 1..23: mol_type = other DNA
gctcagcaggcacctgcctcagc

SEQ ID NO: 163 - DNA, 23 bp, synthetic construct
  misc_feature 1..23: note = Description of sequence: Synthetic oligonucleotide
  source 1..23: mol_type = other DNA
tttcccttcagctaaaataaagg

SEQ ID NO: 164 - DNA, 23 bp, synthetic construct
  misc_feature 1..23: note = Description of sequence: Synthetic oligonucleotide
  source 1..23: mol_type = other DNA
gctgaagggaaataaaaggaaaa

SEQ ID NO: 165 - DNA, 23 bp, synthetic construct
  misc_feature 1..23: note = Description of sequence: Synthetic oligonucleotide
  source 1..23: mol_type = other DNA
acatgtcaatctgtccgttcaca

SEQ ID NO: 166 - DNA, 23 bp, synthetic construct
  misc_feature 1..23: note = Description of sequence: Synthetic oligonucleotide
  source 1..23: mol_type = other DNA
ggaagtgtccagggatgcttccc

SEQ ID NO: 167 - DNA, 23 bp, synthetic construct
  misc_feature 1..23: note = Description of sequence: Synthetic oligonucleotide
  source 1..23: mol_type = other DNA
atgtctgcaggccagatgagggc

SEQ ID NO: 168 - DNA, 23 bp, synthetic construct
  misc_feature 1..23: note = Description of sequence: Synthetic oligonucleotide
  source 1..23: mol_type = other DNA
ggactggagttgcttcatgtaca

SEQ ID NO: 169 - DNA, 23 bp, synthetic construct
  misc_feature 1..23: note = Description of sequence: Synthetic oligonucleotide
  source 1..23: mol_type = other DNA
ggaggtcagaaatagggggtcca

SEQ ID NO: 170 - DNA, 23 bp, synthetic construct
  misc_feature 1..23: note = Description of sequence: Synthetic oligonucleotide
  source 1..23: mol_type = other DNA
ctcctggaccccctatttctgac

SEQ ID NO: 171 - DNA, 23 bp, synthetic construct
  misc_feature 1..23: note = Description of sequence: Synthetic oligonucleotide
  source 1..23: mol_type = other DNA
gaaagggggtggggggagtttgc

SEQ ID NO: 172 - DNA, 23 bp, synthetic construct
  misc_feature 1..23: note = Description of sequence: Synthetic oligonucleotide
  source 1..23: mol_type = other DNA
gccagagccggggtgtgcagacg

SEQ ID NO: 173 - RNA, 228 nt, unidentified
  misc_feature 1..228: note = Description of Unknown: sgRNA sequence
  variation 206..228: note = n = a, c, u, g, unknown or other
  source 1..228: mol_type = other RNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacnnnnnnnnnnnnnnnnnnnnnnn

SEQ ID NO: 174 - RNA, 224 nt, synthetic construct
  misc_feature 1..224: note = Description of Artificial Sequence: Synthetic polynucleotide
  variation 206..224: note = n is a, c, u, g, unknown or other
  source 1..224: mol_type = other RNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattgttcctctccaattctgcacaagaaagttgcagaacccgaatagactaatgaaggaatgcaacnnnnnnnnnnnnnnnnnnn

SEQ ID NO: 175 - RNA, 179 nt, synthetic construct
  misc_feature 1..179: note = Description of Artificial Sequence: Synthetic polynucleotide
  variation 161..179: note = n is a, c, u, g, unknown or other
  source 1..179: mol_type = other RNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnn

SEQ ID NO: 176 - RNA, 177 nt, synthetic construct
  misc_feature 1..177: note = Description of Artificial Sequence: Synthetic polynucleotide
  variation 159..177: note = n is a, c, u, g, unknown or other
  source 1..177: mol_type = other RNA
gcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnn

SEQ ID NO: 177 - protein, 14 aa, unidentified
  REGION 1..14: note = Description of Unknown: Cas14 sequence
GIDVGVKSPLVCAI

SEQ ID NO: 178 - protein, 14 aa, Acidaminococcus sp.
GIDRGERNLIYITV

SEQ ID NO: 179 - protein, 14 aa, unidentified
  REGION 1..14: note = Description of Unknown: Lachnospiraceae bacterium sequence
GIDRGERNLLYIVV

SEQ ID NO: 180 - protein, 14 aa, Francisella tularensis
SIDRGERHLAYYTL

SEQ ID NO: 181 - protein, 14 aa, Moraxella bovoculi
GIDRGERHLLYLTV

SEQ ID NO: 182 - protein, 14 aa, Desulfurobacterium thermolithotrophum
GVDLGLRNLAVVST

SEQ ID NO: 183 - protein, 13 aa, unidentified
  REGION 1..13: note = Description of Unknown: Cas14 sequence
TVQMENLESMKRK

SEQ ID NO: 184 - protein, 13 aa, Acidaminococcus sp.
VVVLENLNFGFKS

SEQ ID NO: 185 - protein, 13 aa, unidentified
  REGION 1..13: note = Description of Unknown: Lachnospiraceae bacterium sequence
VIALEDLNSGFKN

SEQ ID NO: 186 - protein, 13 aa, Francisella tularensis
IVVFEDLNFGFKR

SEQ ID NO: 187 - protein, 13 aa, Moraxella bovoculi
IVVLEDLNFGFKR

SEQ ID NO: 188 - protein, 13 aa, Desulfurobacterium thermolithotrophum
VIVLEKLKGIRKR

SEQ ID NO: 189 - protein, 18 aa, unidentified
  REGION 1..18: note = Description of Unknown: Cas14 sequence
NADYNAALNISNPKLKST

SEQ ID NO: 190 - protein, 18 aa, Acidaminococcus sp.
DADANGAYHIALKGQLLL

SEQ ID NO: 191 - protein, 18 aa, unidentified
  REGION 1..18: note = Description of Unknown: Lachnospiraceae bacterium sequence
NADANGAYNIARKVLWAI

SEQ ID NO: 192 - protein, 18 aa, Francisella tularensis
DADANGAYHIGLKGLMLL

SEQ ID NO: 193 - protein, 18 aa, Moraxella bovoculi
NADANGAYHIALKGLWLL

SEQ ID NO: 194 - protein, 18 aa, Desulfurobacterium thermolithotrophum
NADLNASRNIVKNYLASL

SEQ ID NO: 195 - protein, 24 aa, unidentified
  REGION 1..24: note = Description of Unknown: Cas14 sequence
SVEHYLSDVCYTRAAELFKNAAIA

SEQ ID NO: 196 - protein, 29 aa, Acidaminococcus sp.
HENALLRSFDKFTTYFSGFYENRKNVFSA

SEQ ID NO: 197 - protein, 30 aa, unidentified
  REGION 1..30: note = Description of Unknown: Lachnospiraceae bacterium sequence
KDEIALVNSFNGFTTAFTGFFDNRENMFSE

SEQ ID NO: 198 - protein, 30 aa, Francisella tularensis
DEALEIIKSFKGWTTYFKGFHENRKNVYSS

SEQ ID NO: 199 - protein, 30 aa, Moraxella bovoculi
SPKLAHLAHFEKFSTYFTGFHDNRKNMYSD

SEQ ID NO: 200 - protein, 30 aa, Butyrivibrio sp.
EEDYNALESFRNFYTYFTSYNKVRENLYSD

SEQ ID NO: 201 - protein, 30 aa, Helcococcus kunzii
EEDLEGLNLYSKFTTRLKNFWETRKNVFTD

SEQ ID NO: 202 - protein, 30 aa, Lachnospira pectinoschiza
EKALETIALFKGFTTYFTDYFNIRKNMFKE

SEQ ID NO: 203 - protein, 30 aa, Pseudobutyrivibrio ruminis
DTDYKALDSFSNFYTYFSSYNEVRKNLYSD

SEQ ID NO: 204 - protein, 30 aa, Pseudobutyrivibrio xylanivorans
DADNNALDSFSNFYTYFSSYNEVRKNLYSD

SEQ ID NO: 205 - DNA, 30 bp, synthetic construct
  misc_feature 1..30: note = Description of sequence: Synthetic oligonucleotide
  source 1..30: mol_type = other DNA
tttactccctatcagtgatagagaacgtat

SEQ ID NO: 206 - DNA, 44 bp, Homo sapiens
  source 1..44: mol_type = genomic DNA
tgtttgggaggtcagaaatagggggtccaggagcaaactccccc

SEQ ID NO: 207 - DNA, 44 bp, synthetic construct; identical to SEQ ID NO: 206
  misc_feature 1..44: note = Description of sequence: Synthetic oligonucleotide
  source 1..44: mol_type = other DNA

SEQ ID NO: 208 - DNA, 43 bp, synthetic construct
  misc_feature 1..43: note = Description of sequence: Synthetic oligonucleotide
  source 1..43: mol_type = other DNA
tgtttggaggtcagaaatagggggtccaggagcaaactccccc

SEQ ID NO: 209 - DNA, 33 bp, synthetic construct
  misc_feature 1..33: note = Description of sequence: Synthetic oligonucleotide
  source 1..33: mol_type = other DNA
tgtttgggagggggtccaggagcaaactccccc

SEQ ID NO: 210 - DNA, 41 bp, synthetic construct
  misc_feature 1..41: note = Description of sequence: Synthetic oligonucleotide
  source 1..41: mol_type = other DNA
tgtttgggtcagaaatagggggtccaggagcaaactccccc

SEQ ID NO: 211 - DNA, 42 bp, synthetic construct
  misc_feature 1..42: note = Description of sequence: Synthetic oligonucleotide
  source 1..42: mol_type = other DNA
tgtttggggtcagaaatagggggtccaggagcaaactccccc

SEQ ID NO: 212 - DNA, 44 bp, synthetic construct
  misc_feature 1..44: note = Description of sequence: Synthetic oligonucleotide
  source 1..44: mol_type = other DNA
tgtttgggaggtcagaaataggtggtccaggagcaaactccccc

SEQ ID NO: 213 - DNA, 44 bp, synthetic construct
  misc_feature 1..44: note = Description of sequence: Synthetic oligonucleotide
  source 1..44: mol_type = other DNA
tgtttgggaggtaagaaatagggggtccaggagcaaactccccc

SEQ ID NO: 214 - DNA, 40 bp, synthetic construct
  misc_feature 1..40: note = Description of sequence: Synthetic oligonucleotide
  source 1..40: mol_type = other DNA
tgtttggtcagaaatagggggtccaggagcaaactccccc

SEQ ID NO: 215 - DNA, 43 bp, synthetic construct
  misc_feature 1..43: note = Description of sequence: Synthetic oligonucleotide
  source 1..43: mol_type = other DNA
tgtttgggggtcagaaatagggggtccaggagcaaactccccc

SEQ ID NO: 216 - DNA, 35 bp, synthetic construct
  misc_feature 1..35: note = Description of sequence: Synthetic oligonucleotide
  source 1..35: mol_type = other DNA
tgtttgggatagggggtccaggagcaaactccccc

SEQ ID NO: 217 - DNA, 37 bp, synthetic construct
  misc_feature 1..37: note = Description of sequence: Synthetic oligonucleotide
  source 1..37: mol_type = other DNA
tgtttgggaaatagggggtccaggagcaaactccccc

SEQ ID NO: 218 - DNA, 44 bp, synthetic construct
  misc_feature 1..44: note = Description of sequence: Synthetic oligonucleotide
  source 1..44: mol_type = other DNA
tgtttgggcggtcagaaatagggggtccaggagcaaactccccc

SEQ ID NO: 219 - DNA, 28 bp, Homo sapiens
  source 1..28: mol_type = genomic DNA
gtttgggaggtcagaaatagggggtcca

SEQ ID NO: 220 - DNA, 28 bp, synthetic construct
  misc_feature 1..28: note = Description of sequence: Synthetic oligonucleotide
  source 1..28: mol_type = other DNA
gtttgggaggtcagaaatagggggtcca

SEQ ID NO: 221 - DNA, 28 bp, synthetic construct
  misc_feature 1..28: note = Description of sequence: Synthetic oligonucleotide
  source 1..28: mol_type = other DNA
gtttggggggtcagaaatagggggtcca

SEQ ID NO: 222 - DNA, 28 bp, synthetic construct
  misc_feature 1..28: note = Description of sequence: Synthetic oligonucleotide
  source 1..28: mol_type = other DNA
gtttggggggtcggaaatagggggtcca

SEQ ID NO: 223 - DNA, 28 bp, synthetic construct
  misc_feature 1..28: note = Description of sequence: Synthetic oligonucleotide
  source 1..28: mol_type = other DNA
gtttggggggtcggaaatggggggtcca

SEQ ID NO: 224 - DNA, 28 bp, synthetic construct
  misc_feature 1..28: note = Description of sequence: Synthetic oligonucleotide
  source 1..28: mol_type = other DNA
gtttggggggtcgggaatggggggtcca

SEQ ID NO: 225 - DNA, 28 bp, synthetic construct
  misc_feature 1..28: note = Description of sequence: Synthetic oligonucleotide
  source 1..28: mol_type = other DNA
gtttggggggtcagaaatggggggtcca

SEQ ID NO: 226 - DNA, 28 bp, synthetic construct
  misc_feature 1..28: note = Description of sequence: Synthetic oligonucleotide
  source 1..28: mol_type = other DNA
gtttgggaggtcacaaatagggggtcca

SEQ ID NO: 227 - DNA, 28 bp, synthetic construct
  misc_feature 1..28: note = Description of sequence: Synthetic oligonucleotide
  source 1..28: mol_type = other DNA
gtttggggggtcgggaatagggggtcca

SEQ ID NO: 228 - DNA, 28 bp, Homo sapiens
  source 1..28: mol_type = genomic DNA
gcaaactccccccaccccctttccaaag

SEQ ID NO: 229 - DNA, 28 bp, synthetic construct
  misc_feature 1..28: note = Description of sequence: Synthetic oligonucleotide
  source 1..28: mol_type = other DNA
gcaaactccccccaccccctttccaaag

SEQ ID NO: 230 - DNA, 28 bp, synthetic construct
  misc_feature 1..28: note = Description of sequence: Synthetic oligonucleotide
  source 1..28: mol_type = other DNA
gcaaactccccccaccccctctccaaag

SEQ ID NO: 231 - DNA, 28 bp, synthetic construct
  misc_feature 1..28: note = Description of sequence: Synthetic oligonucleotide
  source 1..28: mol_type = other DNA
gcaaactccccccaccccccttccaaag

SEQ ID NO: 232 - DNA, 28 bp, synthetic construct
  misc_feature 1..28: note = Description of sequence: Synthetic oligonucleotide
  source 1..28: mol_type = other DNA
gcaaactccccccaccccccctccaaag

SEQ ID NO: 233 - DNA, 28 bp, synthetic construct
  misc_feature 1..28: note = Description of sequence: Synthetic oligonucleotide
  source 1..28: mol_type = other DNA
gcaaactcccctcaccccctttccaaag

SEQ ID NO: 234 - DNA, 28 bp, synthetic construct
  misc_feature 1..28: note = Description of sequence: Synthetic oligonucleotide
  source 1..28: mol_type = other DNA
gcaaactccccccaccccccccccaaag

SEQ ID NO: 235 - DNA, 54 bp, Homo sapiens
  source 1..54: mol_type = genomic DNA
gtagctgtttgggaggtcagaaatagggggtccaggagcaaactccccccaccc

SEQ ID NO: 236 - DNA, 54 bp, synthetic construct; identical to SEQ ID NO: 235
  misc_feature 1..54: note = Description of sequence: Synthetic oligonucleotide
  source 1..54: mol_type = other DNA

SEQ ID NO: 237 - DNA, 43 bp, synthetic construct
  misc_feature 1..43: note = Description of sequence: Synthetic oligonucleotide
  source 1..43: mol_type = other DNA
gtagctgtttgggagggggtccaggagcaaactccccccaccc

SEQ ID NO: 238 - DNA, 47 bp, synthetic construct
  misc_feature 1..47: note = Description of sequence: Synthetic oligonucleotide
  source 1..47: mol_type = other DNA
gtagctgtttgggaaatagggggtccaggagcaaactccccccaccc

SEQ ID NO: 239 - DNA, 40 bp, synthetic construct
  misc_feature 1..40: note = Description of sequence: Synthetic oligonucleotide
  source 1..40: mol_type = other DNA
gtagctgtttggggggtccaggagcaaactccccccaccc

SEQ ID NO: 240 - DNA, 52 bp, synthetic construct
  misc_feature 1..52: note = Description of sequence: Synthetic oligonucleotide
  source 1..52: mol_type = other DNA
gtagctgtttggggtcagaaatagggggtccaggagcaaactccccccaccc

SEQ ID NO: 241 - DNA, 53 bp, synthetic construct
  misc_feature 1..53: note = Description of sequence: Synthetic oligonucleotide
  source 1..53: mol_type = other DNA
gtagctgtttgggggtcagaaatagggggtccaggagcaaactccccccaccc

SEQ ID NO: 242 - DNA, 51 bp, synthetic construct
  misc_feature 1..51: note = Description of sequence: Synthetic oligonucleotide
  source 1..51: mol_type = other DNA
gtagctgtttgggtcagaaatagggggtccaggagcaaactccccccaccc

SEQ ID NO: 243 - DNA, 45 bp, synthetic construct
  misc_feature 1..45: note = Description of sequence: Synthetic oligonucleotide
  source 1..45: mol_type = other DNA
gtagctgtttgggatagggggtccaggagcaaactccccccaccc

SEQ ID NO: 244 - DNA, 53 bp, synthetic construct
  misc_feature 1..53: note = Description of sequence: Synthetic oligonucleotide
  source 1..53: mol_type = other DNA
gtagctgtttggaggtcagaaatagggggtccaggagcaaactccccccaccc

SEQ ID NO: 245 - DNA, 46 bp, synthetic construct
  misc_feature 1..46: note = Description of sequence: Synthetic oligonucleotide
  source 1..46: mol_type = other DNA
gtagctgtttgggaatagggggtccaggagcaaactccccccaccc

SEQ ID NO: 246 - DNA, 50 bp, synthetic construct
  misc_feature 1..50: note = Description of sequence: Synthetic oligonucleotide
  source 1..50: mol_type = other DNA
gtagctgtttggtcagaaatagggggtccaggagcaaactccccccaccc

SEQ ID NO: 247 - DNA, 44 bp, synthetic construct
  misc_feature 1..44: note = Description of sequence: Synthetic oligonucleotide
  source 1..44: mol_type = other DNA
gtagctgtttggatagggggtccaggagcaaactccccccaccc

SEQ ID NO: 248 - DNA, 49 bp, synthetic construct
  misc_feature 1..49: note = Description of sequence: Synthetic oligonucleotide
  source 1..49: mol_type = other DNA
gtagctgtttgggagaaatagggggtccaggagcaaactccccccaccc

SEQ ID NO: 249 - DNA, 44 bp, synthetic construct
  misc_feature 1..44: note = Description of sequence: Synthetic oligonucleotide
  source 1..44: mol_type = other DNA
gtagctgtttgggtagggggtccaggagcaaactccccccaccc

SEQ ID NO: 250 - DNA, 44 bp, synthetic construct
  misc_feature 1..44: note = Description of sequence: Synthetic oligonucleotide
  source 1..44: mol_type = other DNA
gtagctgtttgggaggggggtccaggagcaaactccccccaccc

SEQ ID NO: 251 - DNA, 39 bp, synthetic construct
  misc_feature 1..39: note = Description of sequence: Synthetic oligonucleotide
  source 1..39: mol_type = other DNA
gtagctgtttgggggtccaggagcaaactccccccaccc

SEQ ID NO: 252 - RNA, 224 nt, unidentified
  misc_feature 1..224: note = Description of Unknown: tracrRNA sequence
  variation 206..224: note = n is a, c, u, g, unknown or other
  source 1..224: mol_type = other RNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacnnnnnnnnnnnnnnnnnnn

SEQ ID NO: 253 - protein, 5 aa, synthetic construct
  REGION 1..5: note = Description of sequence: Motif
  VARIANT 1: note = Position may also be glutamic acid, lysine or asparagine
  VARIANT 2: note = X is any known amino acid
  VARIANT 3: note = Position may also arginine
  VARIANT 4: note = Position may also be lysine
DXFEN

(19) United States
(12) Patent Application Publication
(10) Pub. No.: US 2026/0035681 A1
(43) Pub. Date: Feb. 5, 2026

(54) SYNTHETIC MINIATURE CRISPR-CAS (CASMINI) SYSTEM FOR EUKARYOTIC GENOME ENGINEERING

(71) Applicant: The Board of Trustees of the Leland Stanford Junior University, Stanford, CA (US)

(72) Inventors: Lei S. Qi, Stanford, CA (US); Xiaoshu Xu, Shanghai (CN)

(73) Assignee: The Board of Trustees of the Leland Stanford Junior University, Stanford, CA (US)

(21) Appl. No.: 19/303,196

(22) Filed: Aug. 18, 2025

Related U.S. Application Data
(63) Continuation of application No. 18/174,552, filed on Feb. 24, 2023, now Pat. No. 12,415,994.
Application No. 18/174,552 is a continuation of application No. PCT/US2021/048362, filed on Aug. 31, 2021.
(60) Provisional application No. 63/073,377, filed on Sep. 1, 2020.
(60) Provisional application No. 63/191,611, filed on May 21, 2021.

(51) Int. Cl. C12N 9/22; C07K 14/31; C12N 15/86

(52) U.S. Cl. CPC C12N 9/22; C07K 14/31; C12N 15/86; C12N 2310/20

(57) ABSTRACT

Provided herein are Cas proteins and guide RNA molecules engineered to exhibit increased activity in eukaryotic cells. The provided Cas proteins and RNA molecules are particularly useful for applications where modulation of eukaryotic nucleic acids with relatively small molecules is advantageous. Also provided are nucleic acids and vectors encoding the disclosed Cas proteins and guide RNA molecules, pharmaceutical compositions including the Cas proteins and guide RNA molecules, and methods for using the disclosed materials.

### CROSS-REFERENCE TO RELATED APPLICATIONS

The present application is a continuation of U.S. application Ser. No. 18/174,552 filed Feb. 24, 2023, which is a continuation of International Patent Application No. PCT/US2021/048362 filed Aug. 31, 2021, which claims priority to U.S. Provisional Application No. 63/073,377 filed Sep. 1, 2020, and U.S. Provisional Application No. 63/191,611 filed May 21, 2021, the full disclosures of which are incorporated by reference in their entireties for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML inTF-8 text. The electronic document, created on Jan. 12, 2024, is entitled “079445-007620US-1374494_SL_ST26.xml”, and is 409,367 bytes in size.

### BACKGROUND

Techniques involving Clustered Regularly Interspaced Short Palindromic Repeat (CRISPR)-associated (Cas) protein have brought revolutionary capability to genome engineering applications (M. Jinek et al., Science 337, (2012): 816-21; L. Cong et al., Science 339, (2013): 819-23. While Cas nucleases (e.g., Streptococcus pyogenes Cas9, Lachnospiraceae bacterium Cas12a) allow for efficient and specific genome editing, nuclease-deactivated Cas (dCas) molecules fused with transcriptional and epigenome effectors enable targeted regulation of endogenous genes in mammalian cells (L. S. Qi et al., Cell 152, (2013): 1173-83; B. Zetsche et al., Cell 163, (2015): 759-71; Y. E. Tak et al., Nat. Methods 14, (2017): 1163-66; B. P. Kleinstiver et al., Nat. Biotechnol. 37, (2019): 276-82; X. Xu & S. L. Qi, J. Mol. Biol. 431, (2019): 34-47; D. C. Swarts, J. van der Oost, & M. Jinek, Mol. Cell 66, (2017): 221-33). These systems offer promising approaches to gene therapies against genetic diseases (B. I. Hilton et al., Nat. Biotechnol. 33, (2015): 510-17; T. S. Klann et al., Nat. Biotechnol. 35, (2017): 561-68; C. Fellmann, B. G. Gowen, P. C. Lin, J. A. Doudna, & J. E. Corn, Nat. Rev. Drug Discov. 16, (2017): 89-100). However, their large size usually prohibits applications. For example, adeno-associated virus (AAV) has a limited payload packaging capacity (<4.5 kb), and many Cas effectors or fusion proteins are beyond this limit.

The discovery of naturally occurring Cas effectors, including Cas14 (Cas12f) and Caso, having smaller sizes when compared to Cas9 or Cas12a (usually 1000 to 1500 amino acids) has offered a natural reservoir of compact Cas effectors (L. B. Harrington et al., Science 362, (2018): 839-42; T. Karvelis et al., Nucleic Acids Res. 48, (2020): 5016-23; S. N. Takeda et al., Mol. Cell 81, (2021): 558-70; P. Pausch et al., Science 369, (2020): 330-37) (FIG. 1). For example, the class 2 type V-F system, CRISPR-Cas14 (400-700 amino acids) is a family of exceptionally compact RNA-guided nucleases from uncultivated archaea. The compact Cas14 effector has not, though, been shown useful in mammalian cells (L. B. Harrington et al., Science 362, (2018): 839-42; T. Karvelis et al., Nucleic Acids Res. 48,(2020): 5016-23). Similarly, the reported compact CasΦ has been found to exhibit only moderate activity in eukaryotic cells (P. Pausch et al., Science 369, (2020): 330-37).

In view of these and other challenges associated with the use of many existing CRISPR-Cas systems, there is a need in the art for improved CRISPR-Cas system components, e.g., compact and highly efficient Cas effectors and/or related guide RNA molecules, for genome engineering applications. In particular, there is a need for improved CRISPR-Cas systems capable of sufficient functioning in mammalian cells. The present disclosure addresses this need and provides associated and other advantages.

### BRIEF SUMMARY

In general, provided herein are miniature Cas effectors engineered, for example, from the type V-F Cas14 (529 amino acids) for efficient gene activation and base editing in mammalian cells. In cases where a natural Cas effector fails to work in mammalian cells, the provided engineered Cas effectors, via guide RNA and protein engineering, can exhibit thousands-fold improvements in activation levels of reporter and endogenous genes in these cell types. The engineered Cas effectors can further have high specificity with no detected off-targets, and allow for robust base editing when fused with an adenine base editor, and robust deletion-insertion gene editing. The synthetic materials and related methods disclosed herein thus provide useful tools for broad applications including those in the fields of gene therapy and cell engineering.

In one aspect, the disclosure provides an engineered Clustered Regularly Interspaced Short Palindromic Repeat (CRISPR)-associated (Cas) protein that is functional in eukaryotic cells. The Cas protein includes a modified amino acid sequence that is at least 80% identical to a native amino acid sequence of a wild-type Cas protein. The native amino acid sequence has a length of less than 700 amino acids and includes a (D/E/K/N)X(R/F)(E/K)N (SEQ ID NO: 253) motif. The modified amino acid sequence includes one or more substitutions in the native amino acid sequence. At least one of the one or more substitutions is at a position either (1) within or no more than 30 amino acids upstream or downstream of the (D/E/K/N)X(R/F)(E/K)N (SEQ ID NO: 253) motif, (2) at or no more than 30 amino acids upstream or downstream of position 241 of the native amino acid sequence, (3) at or no more than 30 amino acids upstream or downstream of position 516 of the native amino acid sequence, or (4) having an electrically charged amino acid in the native amino acid sequence.

In another aspect, the disclosure provides a single-guide RNA (sgRNA) including an engineered CRISPR RNA (crRNA/trans-activating CRISPR RNA (tracrRNA) fusion nucleotide sequence that is at least 60% identical to a wild-type crRNA/tracrRNA fusion nucleotide sequence. The wild-type crRNA/tracrRNA fusion nucleotide sequence includes (1) a 3′ region corresponding to an RNA stem-loop hairpin structure, (2) a poly-U region proximate to the 3′ region, and (3) a 5′ poly-G region. The engineered crRNA/tracrRNA fusion nucleotide sequence includes one or more modifications to the wild-type crRNA/tracrRNA fusion nucleotide sequence. The modifications include substitution of at least one U of the poly-U region, e.g., with a G, deletion of at least a portion of the 3′ region, deletion of at least a portion of the 5′ poly-G region, or a combination of any of these modifications.

In another aspect, the disclosure provides a nucleic acid encoding any of the engineered Cas proteins disclosed herein. In another aspect, the disclosure provides a nucleic acid encoding any of the sgRNA molecules disclosed herein.

In another aspect, the disclosure provides a vector including a nucleic acid encoding any of the engineered Cas proteins disclosed herein, a nucleic acid encoding any of the sgRNA molecules disclosed herein, or a combination thereof.

In another aspect, the disclosure provides a system including an sgRNA and any of the engineered Cas proteins disclosed herein. In another aspect, the disclosure provides a Cas protein and any of the sgRNA molecules disclosed herein. In another aspect, the disclosure provides a system including both a nucleic acid encoding an sgRNA, and a nucleic acid encoding any of the engineered Cas proteins disclosed herein. In another aspect, the disclosure provides a system including both a nucleic acid encoding a Cas protein, and a nucleic acid encoding any of the sgRNA molecules disclosed herein.

In another aspect, the disclosure provides a method of modulating one or more target nucleic acids in a cell. The method includes contacting the cell with any of the engineered Cas proteins disclosed herein, any of the sgRNA molecules disclosed herein, any of the nucleic acids disclosed herein, any of the vectors disclosed herein, or any of the systems disclosed herein.

In another aspect, the disclosure provides a pharmaceutical composition. The pharmaceutical composition includes any of the engineered Cas proteins disclosed herein, any of the sgRNA molecules disclosed herein, any of the nucleic acids disclosed herein, any of the vectors disclosed herein, or any of the systems disclosed herein.

In another aspect, the disclosure provides a method of preventing or treating a disorder, e.g., a genetic disorder, in a subject. The method includes administering to the subject an amount of any of the pharmaceutical compositions disclosed herein, where the amount is sufficient to modulate one or more target nucleic acids associated with the disorder.

In another aspect, the disclosure provides a method of treating an infection in a subject. The method includes administering to the subject an amount of any of the pharmaceutical compositions disclosed herein, where the amount is sufficient to modulate one or more target nucleic acids associated with the infection.

### BRIEF DESCRIPTION OF THE DRAWINGS

FIG. 1 illustrates that engineered novel miniature Cas effector (CasMINI) can serve as a tool for RNA-guided targeted genome engineering requiring better delivery and expression.

FIG. 2 presents schematic construct designs for testing dCas14-VPR fusion for CRISPR activation in a TRE3G-GFP HEK293T reporter cell line. Two mutations were introduced in the RuvC domain of Cas14 to generate nuclease-deactivated dCas14. The sgRNA targets the TRE3G promoter with a TTTR PAM.

FIG. 3 presents graphs showing performance of GFP activation by the constructs of FIG. 2 as measured by flow cytometry. Representative histograms of target and non-target groups show the percentages of GFP positive population and that dCas14 fails to function in mammalian cells.

FIG. 4 presents a schematic illustration of strategies for sgRNA engineering (SEQ ID NO: 173). Design 1, G-U swap; Design 2, stem-loop truncation; Design 3, 5′ poly G removal.

FIG. 5 presents a schematic illustration of wild-type Cas14 sgRNA (SEQ ID NO: 252).

FIG. 6 presents a schematic illustration of sgRNA engineering Design 1 of FIG. 4 (SEQ ID NO: 174).

FIG. 7 presents a schematic illustration of sgRNA engineering Design 2 of FIG. 4 (SEQ ID NO: 175).

FIG. 8 presents a schematic illustration of sgRNA engineering Design 3 of FIG. 4 (SEQ ID NO: 176).

FIG. 9 presents graphs showing performance of GFP activation by transfecting the HEK293T TRE3G reporter line with plasmids containing the dCas14-VPR constructs and four targeting sgRNA of FIGS. 4-8, as well as a non-targeting sgRNA. Left, bars represent activated GFP positive percentage; Right, bars represent GFP mean values; Dash line, the mean value of non-targeting sgRNA group. Dots represent three biological replicates. Fold changes are calculated relative to the non-targeting sgRNA.

FIG. 10 presents schematic illustrations of the design of sgRNA plasmids with the designs of FIGS. 4-8 for mammalian expression.

FIG. 11 presents representative flow cytometry scatter plots showing the percentage of GFP positive cells for each of the sgRNA designs of FIGS. 4-8.

FIG. 12 presents schematic illustrations of a library of designs for dCas14-VPR fusions by fusing VPR to the N or C terminus of dCas14, combining different SV40 or c-MYC nuclear localization signals (NLSs), and different linkers (P2A, glycine-serine linker).

FIG. 13 is a graph showing percentage activated GFP values characterizing gene activation activity of the different dCas14-VPR fusions of FIG. 12. Dots represent three biological replicates. Dash line represents the GFP mean value of the non-targeting sgRNA. Values showing the fold of activation normalized to the non-target sgRNA are labeled.

FIG. 14 is a graph showing mean activated GFP values characterizing gene activation activity of the different dCas14-VPR fusions of FIG. 12. Dots represent three biological replicates. Dash line represents the GFP mean value of the non-targeting sgRNA. Values showing the fold of activation normalized to the non-target sgRNA are labeled.

FIG. 15 presents an overview of the iterative protein engineering strategy. The TRE3G-GFP HEK293T cell line is used to measure GFP activation efficiency of dCas14-VPR variants by flow cytometry 48 h post transfection. The best dCas14-VPR variant for GFP activation is used as the starting sequence for the next round of screening.

FIG. 16 shows alignment of Cas14 to reported Cas 12a protein and DtTnpB, with the conserved active residues of three RuvC domains indicated. FIG. 16 discloses SEQ ID NOS 177-194, respectively, in order of appearance.

FIG. 17 is a schematic illustration indicating the positions of residues chosen for mutagenesis for iterative engineering of the dCas 14 protein.

FIG. 18 is a schematic illustration showing dCas14 protein residues targeted for mutagenesis, the double-stranded DNA substrate, the sgRNA, and the binding centers of the dimer.

FIG. 19 presents graphs showing the GFP activation performance of 28 variants from the first screening round of the iterative protein engineering strategy of FIG. 15. Top, GFP mean values. Bottom, GFP percentage values. The GFP activation folds normalized to a non-target sgRNA targeting lacZl are labeled.

FIG. 20 presents representative flow cytometry histograms showing the percentage of GFP positive cells for wild-type dCas14-VPR using non-targeting sgRNA (top) and targeting sgRNA.

FIG. 21 presents representative flow cytometry histograms showing the percentage of GFP positive cells for wild-type dCasMINI-V1-VPR using non-targeting sgRNA (top) and targeting sgRNA.

FIG. 22 presents representative flow cytometry histograms showing the percentage of GFP positive cells for variants with single (red), double (blue), triple (green) and quadruple (beige) mutations. Values show the percentage of GFP positive cells.

FIG. 23 presents graphs showing the GFP activation performance of variants in two libraries from the second screening round of the iterative protein engineering strategy of FIG. 15.

FIG. 24 presents representative flow cytometry histograms showing the percentage of GFP positive cells for wild-type dCasMINI-V2-VPR using non-targeting sgRNA (top) and targeting sgRNA.

FIG. 25 is a graph showing the GFP activation performance of variants from the third and fourth screening rounds of the iterative protein engineering strategy of FIG. 15.

FIG. 26 presents representative flow cytometry histograms showing the percentage of GFP positive cells for wild-type dCasMINI-V3-VPR using non-targeting sgRNA (top) and targeting sgRNA.

FIG. 27 presents representative flow cytometry histograms showing the percentage of GFP positive cells for wild-type dCasMINI-V4-VPR using non-targeting sgRNA (top) and targeting sgRNA.

FIG. 28 is a graph showing the gradual improvement of CasMINI performance in terms of GFP activation. The fold change of each group is calculated by normalizing gene activation to wild-type dCas 14-VPR.

FIG. 29 illustrates sequence alignment between Cas14 and representative Cas12a proteins. The activity-enhancing residues and the conserved motif (D/E)XRKN (SEQ ID NO: 253) are indicated. FIG. 29 discloses SEQ ID NOS 195-204, respectively, in order of appearance.

FIG. 30 presents schematic illustrations of constructs used to test endogenous gene activation by a dCasMINI-VPR system, and confocal microscopy images showing expression and nuclear localization. Cell nuclei are stained using Hoechst 33342. Bars, 20 μm.

FIG. 31 presents results from gene activation of endogenous HBG in HEK293T cells using dCasMINI-VPR with various single sgRNAs. Top, a schematic illustration of the sgRNA distributions and PAMs. Transcriptional start site (TSS) is designated as ‘0’, and the positions of the first ‘T’ in each PAM are labeled. Bottom, fold changes of top sgRNAs are shown.

FIG. 32 presents results from gene activation of endogenous IL1RN in HEK293T cells using dCasMINI-VPR with various single sgRNAs. Top, a schematic illustration of the sgRNA distributions and PAMs. Transcriptional start site (TSS) is designated as ‘0’, and the positions of the first ‘T’ in each PAM are labeled. Bottom, fold changes of top sgRNAs are shown.

FIG. 33 presents results from gene activation of endogenous ASCL1 in HEK293T cells using dCasMINI-VPR with various single sgRNAs. Top, a schematic illustration of the sgRNA distributions and PAMs. Transcriptional start site (TSS) is designated as ‘0’, and the positions of the first ‘T’ in each PAM are labeled. Bottom, fold changes of top sgRNAs are shown.

FIG. 34 presents results from characterization of a library of 10 sgRNAs using dCasMINI-VPR for activating the human endogenous IFNγ gene. The top schematic illustration shows the binding sites and PAMs using by each sgRNA, designed to target within 500 bp of the transcriptional start site (TSS, position 0). The position of the first ‘T’ of each PAM is shown. The bottom diagram shows characterized gene activation activity of all sgRNAs using qPCR. Dots represent biological replicates.

FIG. 35 presents results from characterization of a library of 10 sgRNAs using dCasMINI-VPR for activating the human endogenous CD2 gene. The top schematic illustration shows the binding sites and PAMs using by each sgRNA, designed to target within 500 bp of the transcriptional start site (TSS, position 0). The position of the first ‘T’ of each PAM is shown. The bottom diagram shows characterized gene activation activity of all sgRNAs using immunostaining followed by flow cytometry. Dots represent biological replicates.

FIG. 36 presents results from characterization of a library of 10 sgRNAs using dCasMINI-VPR for activating the human endogenous CXCR4 gene. The top schematic illustration shows the binding sites and PAMs using by each sgRNA, designed to target within 500 bp of the transcriptional start site (TSS, position 0). The position of the first ‘T’ of each PAM is shown. The bottom diagram shows characterized gene activation activity of all sgRNAs using immunostaining followed by flow cytometry. Dots represent biological replicates.

FIG. 37 presents results from characterization of a library of 20 sgRNAs using dCasMINI-VPR for activating the human endogenous IFNγ gene. The top schematic illustration shows the binding sites and PAMs using by each sgRNA, designed to target within 500 bp of the transcriptional start site (TSS, position 0). The position of the first ‘T’ of each PAM is shown. The bottom diagram shows characterized gene activation activity of all sgRNAs using qPCR. Dots represent biological replicates.

FIG. 38 presents schematic illustrations of constructs used for dCasMINI-VPR, dCas14-VPR, and sgRNA to compare CasMINI and Cas14.

FIG. 39 presents graphs comparing endogenous IFNγ gene activation by dCasMINI-VPR and dCas14-VPR. Fold changes of improvement of dCasMINI-VPR over dCas14-VPR are shown.

FIG. 40 presents graphs comparing endogenous HBB gene activation by dCasMINI-VPR and dCas14-VPR. Fold changes of improvement of dCasMINI-VPR over dCas14-VPR are shown.

FIG. 41 presents graphs comparing endogenous CD2 gene activation by dCasMINI-VPR and dCas14-VPR. Fold changes of improvement of dCasMINI-VPR over dCas14-VPR are shown.

FIG. 42 presents graphs comparing endogenous CXCR4 gene activation by dCasMINI-VPR and dCas14-VPR. Fold changes of improvement of dCasMINI-VPR over dCas14-VPR are shown.

FIG. 43 presents schematic illustrations of dCas12a-VPR and crRNA used for comparisons of dCasMINI-VPR and LbdCas12a-VPR systems using sgRNAs targeting the same genomic sites. FIG. 43 discloses SEQ ID NO: 205.

FIG. 44 is a graph showing activation of GFP in the dCasMINI-VPR and LbdCas12a-VPR system comparison of FIG. 43.

FIG. 45 presents graphs showing activation of endogenous genes in the dCasMINI-VPR and LbdCas12a-VPR system comparison of FIG. 43.

FIG. 46 is a graph showing RNA-seq data of cell samples transfected with the targeting sgRNA vs. the non-targeting sgRNA and dCasMINI-VPR from the comparison of FIG. 43. Mean values of two biological replicates are plotted. The data points for GFP transcripts are labeled.

FIG. 47 is a graph showing RNA-seq data of cell samples transfected with the targeting sgRNA vs. the non-targeting sgRNA and dCas12a-VPR from the comparison of FIG. 43. Mean values of two biological replicates are plotted. The data points for GFP transcripts are labeled.

FIG. 48 presents graphs showing comparisons of biological replicates for each condition tested in the experiment of FIGS. 43-47. From top left to bottom right, dCasMINI-VPR+non-targeting sgRNA, dCasMINI-VPR+targeting sgRNA, dCas12a-VPR+non-targeting sgRNA, dCas12-VPR+targeting sgRNA. The calculated Pearson Correlation for each condition is shown.

FIG. 49 presents graphs showing correlation of side-by-side comparison of dCasMINI-VPR vs. dCas12a-VPR for the non-targeting guide (left) and the targeting guide (right). The calculated Pearson Correlation for each condition is shown.

FIG. 50 is a graph overlaying the data of FIGS. 46 and 47.

FIG. 51 is a graph from the comparison of FIG. 43 showing the distribution of standard deviations for log₁₀[transcripts per million (TPM)+1] values of all genes in RNA sequencing library among non-targeting and targeting replicates for each gene for dCasMINI-VPR and LbdCas12a-VPR, respectively.

FIG. 52 illustrates a comparison of base editing activity using dCasMINI or dCas12a fused to the same ABE8e editor. The schematic diagram shows the constructs used in the experiments. The graph shows the percentage of A⋅T to G⋅C conversion observed when using dCas12a-ABE and dCasMINI-ABE for selected genomic sites. NT, non-targeting sgRNA or crRNA. T, T1, T2, targeting sgRNAs or crRNAs.

FIG. 53 illustrates the silencing of GFP reporter genes using dCasMINI fused to a transcriptional repressor KRAB. The schematic diagrams show the constructs used in the experiments. The graph shows that repression activity observed with the dCasMINI-KRAB construct is comparable to that observed with dCas12a-KRAB.

FIG. 54 presents graphs showing the percentage of gene editing indels, including deletions, insertions, and substitutions, observed when using systems including one of three provided Cas proteins and one of eleven different sgRNAs.

FIG. 55 shows representative sequences from the genomic region targeted by sgVEGFA05 and the corresponding percentages of the sgRNA in the gene editing tests of FIG. 54. FIG. 55 discloses SEQ ID NOS 206-215, 206, 210-211, 215, 208, 213, 216, 212, 206, 209, 210, 217, 215, and 218, respectively, in order of appearance.

FIG. 56 presents schematic illustrations of constructs for four designs by fusing TadA-8e (TadA*) to dCasMINI at the N terminus without (Design 1) or with mCherry (Design 2), fusing TadA* to dCasMINI at the C terminus (Design 3), or fusing a heterodimer TadA-TadA* to dCasMINI at the N terminus (Design 4). The construct of sgRNA is shown on the bottom.

FIG. 57 is a graph comparing four dCasMINI-ABE designs for base editing efficiencies at three different genomic sites in HEK293T cells. The data shown are the percentage of reads with A⋅T to G⋅C conversion over the total aligned reads using deep sequencing. Data are representative of three biological replicates. GS0, genomic site 0. Bars represent mean values and points represent two independent biological replicates.

FIG. 58 presents schematic illustrations of constructs for dCasMINI-ABE Design 4 and its sgRNA and constructs for dCas12a-ABE and its crRNA used for side-by-side comparison for base editing.

FIG. 59 is a graph showing base editing activity using dCasMINI-ABE and dCas12a-ABE at three genomic sites using sgRNAs or crRNAs targeting the same genomic sites. GS1, genomic site 1. Bars represent mean values, and data represent three biological replicates.

FIG. 60 is a graph showing base editing efficiencies in HEK293T cells of more genomic sites with dCasMINI-ABE Design 4, including two sites in the IFNγ locus, three sites in the HBB locus, and four sites in the VEGFA locus. The data shown are the percentage of reads with A⋅T to G⋅C conversion over the total aligned reads using deep sequencing. GS1-3, genomic sites 1-3. Bars represent mean values and points represent three independent biological replicates.

FIG. 61 shows raw sequencing reads from deep sequencing using dCasMINI-ABE and sgRNA targeting site 3 in the IFNγ locus or site 4 in the VEGFA locus. The sequenced reads and percentage among the total aligned reads are shown on the right. Representative variants with >0.2% of the total reads generated by CRISPResso2 are shown. FIG. 61 discloses SEQ ID NOS 219-234, respectively, in order of appearance.

FIG. 62 is a graph showing A⋅T to G⋅C conversion base editing frequency in HEK293T cells by dCasMINI-ABE at adenines for five sites. The schematic of the nucleotide position is shown on the top: the ‘R’ in TTTR PAM is position ‘0’. The arrowed boxes represent the observed most efficient A⋅T to G⋅C conversion positions (position 3 and 4). The data shown is the number of reads with A⋅T to G⋅C conversion at a specific position over the total number of reads for A⋅T to G⋅C conversion using deep sequencing. GS1, genomic site 1. Bars represent mean values and data represent three independent biological replicates.

FIG. 63 presents schematic illustrations of constructs encoding the nuclease active CasMINI and its sgRNA for gene editing, and a table showing three CasMINI variants tested. All experiments used sgRNA Design 2.

FIG. 64 is a graph showing indel activity of each CasMINI variant at four sites of the VEGFA locus measured by deep sequencing in HEK293T cells. The data using a non-targeting (NT) sgRNA is shown as a representative negative control. The dotted line shows the basal indel level detected from wild type HEK293T cells. Bars represent mean values, and data represent three independent biological replicates.

FIG. 65 is a graph showing indel activity of the wild type Cas12f, CasMINI-V2, and CasMINI-V3.1 at two sites of the HBB and IFNγ loci in HEK293T cells. The dotted line shows the data using a sgNT as a representative negative control. Bars represent mean values and data represent three independent biological replicates.

FIG. 66 shows raw sequencing reads from deep sequencing using the wild type Cas12f, CasMINI-V2, CasMINI-V3.1, CasMINI-V4 by targeting the site 3 in the VEGFA locus. The sequenced reads and percentage among the total aligned reads are shown on the right. Representative variants with >0.2% of the total reads generated by CRISPResso2 are shown. FIG. 66 discloses SEQ ID NOS 235-241, 236, 242-244, 238, 237, 240, 245-246, 236, 242, 237, 240, 244, 247, 243, 246, 248-249, 241, 250, 238, 251, 236, 242, 250, and 237, respectively, in order of appearance.

FIG. 67 presents a series of graphs showing the largest indel length during genome editing over eight distinct sites (except for V4 which has four active sites). The data represent the percentage of aligned reads with an insertion or deletion of the given length.

FIG. 68 presents a series of graphs showing indel activity at each nucleotide position during genome editing over eight distinct sites (except for V4 which has four active sites). The data represent the percentage of total reads with a deletion at the position. The schematic on the top show the PAM (4 bp) and the spacer (23 bp), which is aligned to each nucleotide position. The ‘R’ in TTTR PAM is position ‘0’.

FIG. 69 presents an illustration of the engineering, features, and applications of the provided Cas system.

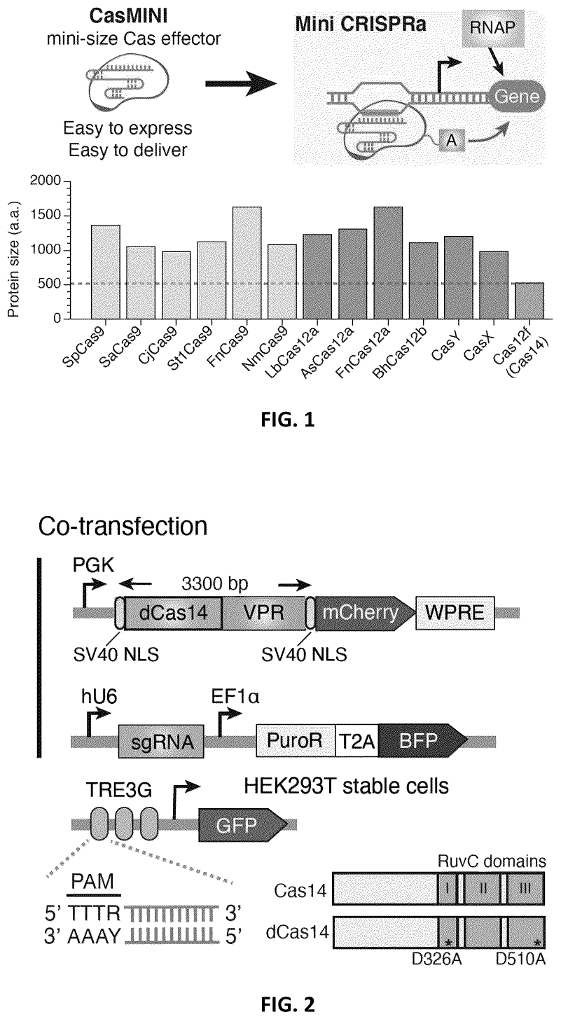

### DETAILED DESCRIPTION

Provided herein are materials and methods involving synthetic compact, efficient, and specific genome engineering systems that advantageously are highly effective in mammalian cells. The provided systems have been developed through particular improvement strategies involving, for example, optimization of the single guide RNA (sgRNA) design and targeted Cas protein engineering (M. T. Reetz & J. D. Carballeira, Mat. Protoc. 2, (2007): 891-903; G. Qu, A. Li, C. G Acevedo-Rocha, Z. Sun, & M. T. Reetz, Angew Chem Int. Ed. Engl. 59, (2020): 13204-31; X. Xu et al., Chembiochem 17 (2016): 56-64). Many of the optimization and engineering approaches described herein use principles not previously appreciated or implemented. As a result of these approaches, the provided variants can, for example, efficiently activate reporter and endogenous gene expression in eukaryotic, e.g., mammalian, cells. Notably, the provided systems exhibit 2 to 3 logs of improvement over the wild-type Cas14, outperform the type V-A dCas12a system, and are specific in mammalian cells without detectable off-targets. Further, when fused to an adenine base editor, the systems can allow robust conversion of A⋅T to G⋅C. Thus, the systems and processes disclosed herein provide useful tools for a variety of genome engineering applications, including those within eukaryotic cells and/or those that require compact effector sizes for delivery and function.

Certain embodiments provide an engineered miniature Cas effector, named CasMINI, derived from the naturally occurring type V-F Cas12f (Cas14) system, which was only 529 amino acids compared to 1,368 amino acids of commonly used SpCas9 or 1,228 amino acid of LbCas12a. While the natural Cas14 shows no activity in mammalian cells, synthetic CasMINI engineered via iterative protein screening and optimized sgRNA designs exhibits highly efficient activation of target genes. The efficiency of activation is, for example, comparable to or better than that of the Cas12a system. Also beneficially, no significant detectable off-target activity is detected with the CasMINI system, and the system has been shown useful for other genetic engineering applications such as base editing.

Despite the rapid advancement of CRISPR-Cas systems, a biotechnological challenge for cell engineering or in vivo delivery remains, particular with respect to eukaryotic cells, due to the large size of Cas effectors. The engineered compact molecules disclosed herein have greatly reduced sizes, making them more suitable than other Cas effectors for medical treatment methods. For example, the small size of the CasMINI system makes it compatible with adeno-associated virus (AAV) packaging and can enhance delivery efficiency when using lipid nanoparticles to carry mRNA payloads. Furthermore, the small size advantageously makes the provided materials less immunogenic when compared to large protein payloads. Moreover, the general RNA and protein engineering approach used in the work also makes it possible to engineer Cas effectors, e.g., Cas14/Cas12f effectors, from other bacterial species.

### I. Cas Proteins

In one aspect, an engineered Cas protein is provided. The engineered Cas protein disclosed herein provides surprising improvements in its ability to function in eukaryotic cells. The engineered Cas protein has a modified amino acid sequence that is at least 80% identical to a native amino acid sequence of a wild-type Cas protein. The modified amino acid sequence of the engineered Cas protein can be, for example, at least 80%, at least 82%, at least 84%, at least 86%, at least 88%, at least 90%, at least 91%, at least 92%, at least 93%, at least 94%, at least 95%, at least 96%, at least 97%, at least 98%, or at least 99% identical to a native amino acid sequence of a wild-type Cas protein.

CRISPR systems are generally divided into two classes, with class 1 systems using a complex of multiple Cas proteins to degrade foreign nucleic acids, and class 2 systems using a single, generally larger, Cas protein for the same purpose. Class 1 is divided into types I, III, and IV, and class 2 is divided into types II, V, and VI. In some embodiments, the wild-type Cas protein that the engineered Cas protein is a modification of is a type V Cas protein. The wild-type Cas protein can be, for example, a type V-A Cas protein, a type V-B Cas protein, a type V-C Cas protein, a type V-D Cas protein, a type V-E Cas protein, a type V-F Cas protein, a type V-G Cas protein, a type V-H Cas protein, a type V-I Cas protein, a type V-J Cas protein, a type V-K Cas protein, or a type V-U Cas protein. In some embodiments, the wild-type Cas protein is a type V-J protein, such as a wild-type CasΦ (Cas 12J) protein.

In some embodiments, the wild-type Cas protein is a type V-F Cas protein such as Cas14. In some embodiments, the wild-type Cas protein is a Cas14 (Cas12f) protein having the native amino sequence of SEQ ID NO: 1. Cas14 proteins are Type V subtype F RNA-guided nucleic acid-binding proteins that can be targeted to DNA and/or RNA, and are much smaller than typical CRISPR effectors, ranging in size from about 400 amino acids to about 700 amino acids. At least 24 different Cas14 variants have been identified that cluster into three subgroups, Cas14a, Cas14b, and Cas14c, based on sequence comparison, all of which share a predicted RuvC nuclease domain characteristic of type V CRISPR-Cas DNA-targeting enzymes. The small size of Cas14 proteins allows Cas14 proteins and effector domain fusions thereof to be paired with a CRISPR array encoding multiple guide RNAs while remaining under the packaging size limit of the versatile adeno-associated virus (AAV) delivery vehicle for primary cell and in vivo delivery. Targeted AAV delivery of dCas14 systems to cells can allow for long-term expression of a corrective payload that avoids permanent genetic modifications or frequent re-administration, complementing other nucleic acid-targeting technologies such as DNA nuclease editing or antisense oligonucleotides. CRISPR-Cas14 and engineered variants such as dCas14 allow for flexible nucleic acid engineering, regulation of gene expression, and therapeutics, expanding the genome editing and regulation toolbox.

In some embodiments, the wild-type Cas protein that the engineered Cas protein is a modification of has a native amino acid sequence with a length of less than 700 amino acids. This relatively small size provides several advantages to the provided engineered Cas protein. For example, the small size can allow the Cas protein to be delivered to a host cell, e.g., a cell of a human patient, via a single adeno-associated virus delivery system that would be otherwise incapable of delivering a larger protein. The native amino acid sequence can have a length that is, for example, between 500 amino acids and 700 amino acids, e.g., between 500 amino acids and 620 amino acids, between 540 amino acids and 660 amino acids, between 560 amino acids and 680 amino acids, or between 580 amino acids and 700 amino acids. In terms of upper limits, the native amino acid sequence can have a length that is less than 700 amino acids, e.g., less than 680 amino acids, less than 660 amino acids, less than 640 amino acids, less than 620 amino acids, less than 600 amino acids, less than 580 amino acids, less than 560 amino acids, less than 540 amino acids, or less than 520 amino acids. In terms of lower limits, the native amino acid sequence can have a length that is greater than 500 amino acids, e.g., greater than 520 amino acids, greater than 540 amino acid, greater than 560 amino acids, greater than 580 amino acids, greater than 600 amino acids, greater than 620 amino acids, greater than 640 amino acids, greater than 660 amino acids, or greater than 700 amino acids. Larger lengths, e.g., greater than 700 amino acids, and smaller lengths, e.g., less than 500 amino acids, are also contemplated.

In some embodiments, the modified amino acid sequence of the engineered Cas protein includes one or more substitutions in the native amino acid sequence, where the positions of at least some of these substitutions follow one or more particular rules determined to have surprising advantages for the characteristics of the engineered Cas protein. For example, the particular substitution rules have been selected for their ability to produce engineered Cas proteins capable of functioning within eukaryotic cells. According to these particular rules, all or some of the one or more substitutions in the native amino acid sequence are either (1) within or no more than 30 amino acids downstream of a (D/E/K/N)X(R/F)(E/K)N (SEQ ID NO: 253) motif of the native amino acid sequence, (2) at or no more than 30 amino acids upstream or downstream of position 241 of the native amino acid sequence, (3) at or no more than 30 amino acids upstream or downstream of position 516 of the native amino acid sequence, or (4) having an electrically charged amino acid in the native amino acid sequence.

In some embodiments, the one or more substitutions in the native amino acid sequence include substitutions in one of the above four categories. The one or more substitutions can include substitutions in one category that is either category (1), (2), (3) or (4). The one or more substitutions can consist of substitutions in one category that is either category (1), (2), (3), or (4). In some embodiments, the one or more substitutions are each independently in one of two categories selected from categories (1) and (2), (1) and (3), (1) and (4), (2) and (3), (2) and (4), or (3) and (4). In some embodiments, the one or more substitutions include substitutions in each of the two categories (1) and (2), (1) and (3), (1) and (4), (2) and (3), (2) and (4), or (3) and (4). In some embodiments, the one or more substitutions consist of substitutions in each of the two categories (1) and (2), (1) and (3), (1) and (4), (2) and (3), (2) and (4), or (3) and (4). In some embodiments, the one or more substitutions are each independently in one of the three categories (1), (2) and (3); (1), (2), and (4); (1), (3), and (4); or (2), (3), and (4). In some embodiments, the one or more substitutions include substitutions in each of the three categories (1), (2) and (3); (1), (2), and (4); (1), (3), and (4); or (2), (3), and (4). In some embodiments, the one or more substitutions consist of substitutions in each of the three categories (1), (2) and (3); (1), (2), and (4); (1), (3), and (4); or (2), (3), and (4). In some embodiments, the one or more substitutions include substitutions in each of the four categories.

Further, in some embodiments, additional substitution rules are followed wherein all or some of the one or more amino acid substitutions in the native amino acid sequence are to a small amino acid, e.g., arginine (R), alanine (A), serine(S), or glycine (G). In some embodiments, the one or more substitutions includes a substitution to R. In some embodiments, each of the one or more substitutions is a substitution to R. In some embodiments, the one or more substitutions includes a substitution to A. In some embodiments, each of the one or more substitutions is a substitution to A. In some embodiments, the one or more substitutions includes a substitution to S. In some embodiments, each of the one or more substitutions is a substitution to S. In some embodiments, the one or more substitutions includes a substitution to G. In some embodiments, each of the one or more substitutions is a substitution to G. In some embodiments, the one or more substitutions include substitutions to the two amino acids R and A, R and S, R and G, A and S, A and G, or S and G. In some embodiments, the one or more substitutions consist of substitutions that are each independently to one of the two amino acids R and A, R and S, R and G, A and S, A and G, or S and G. In some embodiments, the one or more substitutions consist of substitutions to each of the two amino acids R and A, R and S, R and G, A and S, A and G, or S and G. In some embodiments, the one or more substitutions include substitutions to the three amino acids R, A, and S; R, A, and G; R, S, and G; or A, S, and G. In some embodiments, the one or more substitutions consist of substitutions that are each independently to one of the three amino acids R, A, and S; R, A, and G; R, S, and G; or A, S, and G. In some embodiments, the one or more substitutions consist of substitutions to each of the three amino acids R, A, and S; R, A, and G; R, S, and G; or A, S, and G. In some embodiments, the one or more substitutions include substitutions to the four amino acids R, A, S, and G. In some embodiments, the one or more substitutions consist of substitutions that are each independently to one of the four amino acids R, A, S, and G. In some embodiments, the one or more substitutions consist of substitutions to each of the four amino acids R, A, S, and G.

In some embodiments, the native amino acid sequence includes a (D/E/K/N)X(R/F)(E/K)N (SEQ ID NO: 253) motif, and the modified amino acid sequence includes one or more substitutions at positions within or no more than 30 amino acids upstream or downstream of the motif. The modified amino acid sequence can include, for example, one, two, three, four, five, six, seven, eight, nine, ten, or more than ten substitutions within or no more than 30 amino acids upstream or downstream of the motif. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of the motif. In some embodiments, at least one of the one or more substitutions within or no more than 30 amino acids upstream or downstream of the motif is to an R, A, S, or G. In some embodiments, each of the one or more substitutions within or no more than 30 amino acids upstream or downstream of the motif is independently to an R, A, S, or G. In some embodiments, all of the substitutions to the native amino acid sequence are at positions within or no more than 30 amino acids upstream or downstream of the motif.

The one or more substitutions at positions within or no more than 30 amino acids upstream or downstream of the (D/E/K/N)X(R/F)(E/K)N (SEQ ID NO: 253) motif of the native amino acid sequence can include, for example, one or more substitutions at positions selected from positions 143, 147, 151, and 154 of the native amino acid sequence. In some embodiments, e.g., when the native amino acid sequence is the sequence of SEQ ID NO: 1, the one or more substitutions include substitutions are at one or more positions selected from D143, T147, E151, and K154. In some embodiments, e.g., when the native amino acid sequence is the sequence of SEQ ID NO: 1, the one or more substitutions include one or more substitutions selected from D143R, T147R, E151R, and K154R.

In some embodiments, the modified amino acid sequence includes one or more substitutions at or no more than 30 amino acids upstream or downstream of position 241 of the native amino acid sequence. The modified amino acid sequence can include, for example, one, two, three, four, five, six, seven, eight, nine, ten, or more than ten substitutions within or no more than 30 amino acids upstream or downstream of position 241. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of position 241. In some embodiments, at least one of the one or more substitutions within or no more than 30 amino acids upstream or downstream of position 241 is to an R, A, S, or G. In some embodiments, each of the one or more substitutions within or no more than 30 amino acids upstream or downstream of position 241 is independently to an R, A, S, or G. In some embodiments, all of the substitutions to the native amino acid sequence are at positions within or no more than 30 amino acids upstream or downstream of position 241.

The one or more substitutions at positions within or no more than 30 amino acids upstream or downstream of position 241 of the native amino acid sequence can include, for example, a substitution at positions 241 of the native amino acid sequence. In some embodiments, e.g., when the native amino acid sequence is the sequence of SEQ ID NO: 1, the one or more substitutions include a substitution at position E241. In some embodiments, e.g., when the native amino acid sequence is the sequence of SEQ ID NO: 1, the one or more substitutions include an E241 substitution.

In some embodiments, the modified amino acid sequence includes one or more substitutions at or no more than 30 amino acids upstream or downstream of position 516 of the native amino acid sequence. The modified amino acid sequence can include, for example, one, two, three, four, five, six, seven, eight, nine, ten, or more than ten substitutions within or no more than 30 amino acids upstream or downstream of position 516. At least one of the one or more substitutions to the native amino acid sequence can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of position 516. In some embodiments, at least one of the one or more substitutions within or no more than 30 amino acids upstream or downstream of position 516 is to an R, A, S, or G. In some embodiments, each of the one or more substitutions within or no more than 30 amino acids upstream or downstream of position 516 is independently to an R, A, S, or G. In some embodiments, all of the substitutions to the native amino acid sequence are at positions within or no more than 30 amino acids upstream or downstream of position 516.

The one or more substitutions at positions within or no more than 30 amino acids upstream or downstream of position 516 of the native amino acid sequence can include, for example, one or more substitutions at positions selected from positions 504, 507, 516, 519, 527, and 528 of the native amino acid sequence. In some embodiments, e.g., when the native amino acid sequence is the sequence of SEQ ID NO: 1, the one or more substitutions include substitutions are at one or more positions selected from N504, E507, N516, N519, E527, and E528. In some embodiments, e.g., when the native amino acid sequence is the sequence of SEQ ID NO: 1, the one or more substitutions include one or more substitutions selected from N504R, E507R, N516R, N519R, E527R, and E528R.

In some embodiments, the modified amino acid sequence includes one or more substitutions at positions of the native amino acid sequence having an electrically charged amino acid. The modified amino acid sequence can include, for example, substitutions at positions having an aspartic acid (D), glutamic acid (E), lysine (K), arginine (R), or histidine (H). In some embodiments, at least one of the one or more substitutions for an electrically charged amino acid is to an R, A, S, or G. In some embodiments, each of the one or more substitutions for an electrically charged amino acid is independently to an R, A, S, or G. In some embodiments, all of the substitutions to the native amino acid sequence are at positions having an electrically charged amino acid.

In some embodiments, e.g., when the native amino acid sequence is the sequence of SEQ ID NO: 1, the one or more substitutions at positions having an electrically charged amino include substitutions are at one or more positions selected from K11, K73, D143, E151, K154, E241, D318, K330, K457, E425, E462, E507, E527, and E528. In some embodiments, e.g., when the native amino acid sequence is the sequence of SEQ ID NO: 1, the one or more substitutions include one or more substitutions selected from K11R, K73R, D143R, E151R, K154R, E241R, D318R, K330R, E425N, K457R, E462R, E507R, E527R, and E528R. In some embodiments, the modified amino acid sequence includes a D143R substitution. In some embodiments, the only substitution in the modified amino acid sequence is D143R. In some embodiments, the modified amino acid sequence is the sequence of SEQ ID NO: 2.

In some embodiments, the modified amino acid sequence of the engineered Cas protein includes two substitutions in the native amino acid sequence. In some embodiments, the modified amino acid sequence has exactly two substitutions in the native amino acid sequence. In some embodiments, the modified amino acid sequence includes two substitutions at positions selected from positions 143, 147, 151, 154, 241, 330, 425, 504, 507, 516, 519, 527, and 528. In some embodiments, the modified amino acid sequence has exactly two substitutions, where the exactly two substitutions are at positions selected from positions 143, 147, 151, 154, 241, 330, 425, 504, 507, 516, 519, 527, and 528. In some embodiments, e.g., when the native amino acid sequence is the sequence of SEQ ID NO: 1, the modified amino acid sequence includes two substitutions at positions selected from D143, T147, E151, K154, E241, K330, E425, N504, E507, N516, N519, E527, and E528. In some embodiments, e.g., when the native amino acid sequence is the sequence of SEQ ID NO: 1, the modified amino acid sequence has exactly two substitutions, where the exactly two substitutions are at positions selected from D143, T147, E151, K154, E241, K330, E425, N504, E507, N516, N519, E527, and E528.

In some embodiments, the modified amino acid sequence includes a substitution at position 143 and a substitution at a position selected from positions 147, 151, 154, 241, 330, 425, 504, 507, 516, 519, 527, and 528. In some embodiments, the modified amino acid includes a substitution at position 143 and exactly one other substitution, where the exactly one other substitution is at a position selected from positions 147, 151, 154, 241, 330, 425, 504, 507, 516, 519, 527, and 528. In some embodiments, e.g., when the native amino acid sequence is the sequence of SEQ ID NO: 1, the modified amino acid sequence includes a substitution at position D143 and a substitution at a position selected from positions T147, E151, K154, E241, K330R, E425N, N504, E507, N516, N519, E527, and E528. In some embodiments, e.g., when the native amino acid sequence is the sequence of SEQ ID NO: 1, the modified amino acid includes a substitution at position D143 and exactly one other substitution, where the exactly one other substitution is at a position selected from positions T147, E151, K154, E241, K330R, E425N, N504, E507, N516, N519, E527, and E528.

In some embodiments, e.g., when the native amino acid sequence is the sequence of SEQ ID NO: 1, the modified amino acid includes two substitutions selected from D143R, T147R, E151R, E151A, K154R, E241R, N504R, E507R, N516R, N519R, E527R, and E528R. In some embodiments, e.g., when the native amino acid sequence is the sequence of SEQ ID NO: 1, the modified amino acid includes exactly two substitutions, where the two substitutions are selected from D143R, T147R, E151R, E151A, K154R, E241R, N504R, E507R, N516R, N519R, E527R, and E528R. In some embodiments, e.g., when the native amino acid sequence is the sequence of SEQ ID NO: 1, the modified amino acid includes two substitutions selected from D143R/T147R, D143R/E151R, D143R/E241R, D143R/E425N, D143R/E507R, D143R/N519R, D143R/E527R, D143R/E528R, D143R/R151S, D143/R151G, and D143R/E151A. In some embodiments, e.g., when the native amino acid sequence is the sequence of SEQ ID NO: 1, the modified amino acid includes exactly two substitutions, where the two substitutions are selected from D143R/T147R, D143R/E151R, D143R/E241R, D143R/E425N, D143R/E507R, D143R/N519R, D143R/E527R, D143R/E528R, D143R/R151S, D143/R151G, and D143R/E151A. In some embodiments, the modified amino acid sequence includes a D143R substitution and a T147R substitution. In some embodiments, the only substitutions in the modified amino acid sequence are a D143R substitution and a T147R substitution. In some embodiments, the modified amino acid sequence is the sequence of SEQ ID NO: 3.

In some embodiments, the modified amino acid sequence of the engineered Cas

protein includes three substitutions in the native amino acid sequence. In some embodiments, the modified amino acid sequence has exactly three substitutions in the native amino acid sequence. In some embodiments, the modified amino acid sequence includes three substitutions at positions selected from positions 143, 147, 151, 154, 241, 330, 425, 504, 507, 516, 519, 527, and 528. In some embodiments, the modified amino acid sequence has exactly three substitutions, where the exactly three substitutions are at positions selected from positions 143, 147, 151, 154, 241, 330, 425, 504, 507, 516, 519, 527, and 528. In some embodiments, e.g., when the native amino acid sequence is the sequence of SEQ ID NO: 1, the modified amino acid sequence includes three substitutions at positions selected from D143, T147, E151, K154, E241, K330, E425, N504, E507, N516, N519, E527, and E528. In some embodiments, e.g., when the native amino acid sequence is the sequence of SEQ ID NO: 1, the modified amino acid sequence has exactly three substitutions, where the exactly three substitutions are at positions selected from D143, T147, E151, K154, E241, K330, E425, N504, E507, N516, N519, E527, and E528.

In some embodiments, the modified amino acid sequence includes a substitution at position 143, a substitution at position 147, and a substitution at a position selected from positions 151, 154, 241, 330, 425, 504, 507, 516, 519, 527, and 528. In some embodiments, the modified amino acid includes a substitution at position 143, a substitution at position 147, and exactly one other substitution, where the exactly one other substitution is at a position selected from positions 151, 154, 241, 330, 425, 504, 507, 516, 519, 527, and 528. In some embodiments, e.g., when the native amino acid sequence is the sequence of SEQ ID NO: 1, the modified amino acid sequence includes a substitution at position D143, a substitution at position T147, and a substitution at a position selected from positions E151, K154, E241, K330, E425, N504, E507, N516, N519, E527, and E528. In some embodiments, e.g., when the native amino acid sequence is the sequence of SEQ ID NO: 1, the modified amino acid includes a substitution at position D143, a substitution at position T147, and exactly one other substitution, where the exactly one other substitution is at a position selected from positions E151, K154, E241, K330, E425, N504, E507, N516, N519, E527, and E528.

In some embodiments, e.g., when the native amino acid sequence is the sequence of SEQ ID NO: 1, the modified amino acid includes three substitutions selected from D143R, T147R, E151R, E151A, E151S, E151G, K154R, E241R, K330R, E425N, N504R, E507R, N516R, N519R, E527R, and E528R. In some embodiments, e.g., when the native amino acid sequence is the sequence of SEQ ID NO: 1, the modified amino acid includes exactly three substitutions, where the three substitutions are selected from D143R, T147R, E151R, E151A, E151S, E151G, K154R, E241R, K330R, E425N, N504R, E507R, N516R, N519R, E527R, and E528R. In some embodiments, e.g., when the native amino acid sequence is the sequence of SEQ ID NO: 1, the modified amino acid includes three substitutions selected from D143R/T147R/K330R, D143R/T147R/K154R, D143R/T147R/E241R, D143R/T147R/E507R, D143R/T147R/N519R, D143R/T147R/E527R, D143R/T147R/E528R, D143R/T147R/E151S, D143R/T147R/E151G, and D143R/T147R/E151A. In some embodiments, e.g., when the native amino acid sequence is the sequence of SEQ ID NO: 1, the modified amino acid includes exactly three substitutions, where the three substitutions are selected from D143R/T147R/K330R, D143R/T147R/K154R, D143R/T147R/E241R, D143R/T147R/E507R, D143R/T147R/N519R, D143R/T147R/E527R, D143R/T147R/E528R, D143R/T147R/E151S, D143R/T147R/E151G, and D143R/T147R/E151A. In some embodiments, the modified amino acid sequence includes a D143R substitution, a T147R substitution, and a K330R substitution. In some embodiments, the only substitutions in the modified amino acid sequence are a D143R substitution, a T147R substitution, and a K330R substitution. In some embodiments, the modified amino acid sequence is the sequence of SEQ ID NO: 4.

In some embodiments, the modified amino acid sequence of the engineered Cas protein includes four substitutions in the native amino acid sequence. In some embodiments, the modified amino acid sequence has exactly four substitutions in the native amino acid sequence. In some embodiments, the modified amino acid sequence includes four substitutions at positions selected from positions 143, 147, 151, 154, 241, 330, 425, 504, 507, 516, 519, 527, and 528. In some embodiments, the modified amino acid sequence has exactly four substitutions, where the exactly four substitutions are at positions selected from positions 143, 147, 151, 154, 241, 330, 425, 504, 507, 516, 519, 527, and 528. In some embodiments, e.g., when the native amino acid sequence is the sequence of SEQ ID NO: 1, the modified amino acid sequence includes four substitutions at positions selected from D143, T147, E151, K154, E241, K330, E425, N504, E507, N516, N519, E527, and E528. In some embodiments, e.g., when the native amino acid sequence is the sequence of SEQ ID NO: 1, the modified amino acid sequence has exactly four substitutions, where the exactly four substitutions are at positions selected from D143, T147, E151, K154, E241, K330, E425, N504, E507, N516, N519, E527, and E528.

In some embodiments, the modified amino acid sequence includes a substitution at position 143, a substitution at position 147, a substitution at position 330, and a substitution at a position selected from positions 151, 154, 241, 425, 504, 507, 516, 519, 527, and 528. In some embodiments, the modified amino acid includes a substitution at position 143, a substitution at position 147, a substitution at position 330, and exactly one other substitution, where the exactly one other substitution is at a position selected from positions 151, 154, 241, 425, 504, 507, 516, 519, 527, and 528. In some embodiments, e.g., when the native amino acid sequence is the sequence of SEQ ID NO: 1, the modified amino acid sequence includes a substitution at position D143, a substitution at position T147, a substitution at K330, and a substitution at a position selected from positions E151, K154, E241, E425, N504, E507, N516, N519, E527, and E528. In some embodiments, e.g., when the native amino acid sequence is the sequence of SEQ ID NO: 1, the modified amino acid includes a substitution at position D143, a substitution at position T147, a substitution at position K330, and exactly one other substitution, where the exactly one other substitution is at a position selected from positions E151, K154, E241, E425, N504, E507, N516, N519, E527, and E528.

In some embodiments, e.g., when the native amino acid sequence is the sequence of SEQ ID NO: 1, the modified amino acid includes four substitutions selected from D143R, T147R, E151R, E151A, E151S, E151G, K154R, E241R, K330R, E425N, N504R, E507R, N516R, N519R, E527R, and E528R. In some embodiments, e.g., when the native amino acid sequence is the sequence of SEQ ID NO: 1, the modified amino acid includes exactly four substitutions, where the four substitutions are selected from D143R, T147R, E151R, E151A, E151S, E151G, K154R, E241R, K330R, E425N, N504R, E507R, N516R, N519R, E527R, and E528R. In some embodiments, e.g., when the native amino acid sequence is the sequence of SEQ ID NO: 1, the modified amino acid includes four substitutions selected from D143R/T147R/K330R/E528R, D143R/T147R/K330R/E151A, and D143R/T147R/K330R/E527R. In some embodiments, e.g., when the native amino acid sequence is the sequence of SEQ ID NO: 1, the modified amino acid includes exactly four substitutions, where the four substitutions are selected from D143R/T147R/K330R/E528R, D143R/T147R/K330R/E151A, and D143R/T147R/K330R/E527R. In some embodiments, the modified amino acid sequence includes a D143R substitution, a T147R substitution, a K330R substitution, and an E528R substitution. In some embodiments, the only substitutions in the modified amino acid sequence are a D143R substitution, a T147R substitution, a K330R substitution, and an E528R substitution. In some embodiments, the modified amino acid sequence is the sequence of SEQ ID NO: 5.

In some embodiments, the modified amino acid sequence of the engineered Cas protein includes five substitutions in the native amino acid sequence. In some embodiments, the modified amino acid sequence has exactly five substitutions in the native amino acid sequence. In some embodiments, the modified amino acid sequence includes five substitutions at positions selected from positions 143, 147, 151, 154, 241, 330, 504, 507, 516, 519, 527, and 528. In some embodiments, the modified amino acid sequence has exactly five substitutions, where the exactly five substitutions are at positions selected from positions 143, 147, 151, 154, 241, 330, 504, 507, 516, 519, 527, and 528. In some embodiments, e.g., when the native amino acid sequence is the sequence of SEQ ID NO: 1, the modified amino acid sequence includes five substitutions at positions selected from D143, T147, E151, K154, E241, K330, N504, E507, N516, N519, E527, and E528. In some embodiments, e.g., when the native amino acid sequence is the sequence of SEQ ID NO: 1, the modified amino acid sequence has exactly five substitutions, where the exactly five substitutions are at positions selected from D143, T147, E151, K154, E241, K330, N504, E507, N516, N519, E527, and E528.

In some embodiments, the modified amino acid sequence includes a substitution at position 143, a substitution at position 147, a substitution at position 330, a substitution at position 151, and a substitution at a position selected from positions 154, 241, 504, 507, 516, 519, 527, and 528. In some embodiments, the modified amino acid includes a substitution at position 143, a substitution at position 147, a substitution at position 330, a substitution at position 151, and exactly one other substitution, where the exactly one other substitution is at a position selected from positions 151, 154, 241, 504, 507, 516, 519, 527, and 528. In some embodiments, e.g., when the native amino acid sequence is the sequence of SEQ ID NO: 1, the modified amino acid sequence includes a substitution at position D143, a substitution at position T147, a substitution at K330, a substitution at E151, and a substitution at a position selected from positions K154, E241, E425, N504, E507, N516, N519, E527, and E528. In some embodiments, e.g., when the native amino acid sequence is the sequence of SEQ ID NO: 1, the modified amino acid includes a substitution at position D143, a substitution at position T147, a substitution at position K330, a substitution at E151, and exactly one other substitution, where the exactly one other substitution is at a position selected from positions K154, E241, E425, N504, E507, N516, N519, E527, and E528.

In some embodiments, e.g., when the native amino acid sequence is the sequence of SEQ ID NO: 1, the modified amino acid includes five substitutions selected from D143R, T147R, E151R, E151A, E151S, E151G, K154R, E241R, K330R, N504R, E507R, N516R, N519R, E527R, and E528R. In some embodiments, e.g., when the native amino acid sequence is the sequence of SEQ ID NO: 1, the modified amino acid includes exactly five substitutions, where the five substitutions are selected from D143R, T147R, E151R, E151A, E151S, E151G, K154R, E241R, K330R, N504R, E507R, N516R, N519R, E527R, and E528R. In some embodiments, e.g., when the native amino acid sequence is the sequence of SEQ ID NO: 1, the modified amino acid includes five substitutions selected from D143R/T147R/K330R/E151A/E527R and D143R/T147R/K330R/E151A/E528R. In some embodiments, e.g., when the native amino acid sequence is the sequence of SEQ ID NO: 1, the modified amino acid includes exactly five substitutions, where the five substitutions are selected from D143R/T147R/K330R/E151A/E527R and D143R/T147R/K330R/E151A/E528R. In some embodiments, the modified amino acid sequence includes a D143R substitution, a T147R substitution, a K330R substitution, an E528R substitution, and an E151A substitution. In some embodiments, the only substitutions in the modified amino acid sequence are a D143R substitution, a T147R substitution, a K330R substitution, an E528R substitution, and an E151A substitution. In some embodiments, the modified amino acid sequence is the sequence of SEQ ID NO: 6.

In some embodiments, the engineered Cas protein is further modified such that the Cas protein is a fully or partially nuclease deactivated Cas (dCas) protein. Such alteration or modification includes altering one or more amino acids to inactivate the nuclease activity or the nuclease domain, as well as removing the polypeptide sequence or polypeptide sequences exhibiting nuclease activity, i.e., the nuclease domain, such that the polypeptide sequence or polypeptide sequences exhibiting nuclease activity, i.e. nuclease domain, are absent from the Cas14 protein. Thus, in some embodiments, a nuclease-null Cas14 protein (dCas14) includes polypeptide sequences modified to inactivate nuclease activity or removal of a polypeptide sequence or sequences to inactivate nuclease activity. The dCas14 protein retains the ability to bind to target nucleic acid even though the nuclease activity has been inactivated. Accordingly, the dCas14 protein includes the polypeptide sequence or sequences required for nucleic acid binding. In some embodiments, one or both of D326 and D510 are substituted with an amino acid that reduces, substantially eliminates, or eliminates nuclease activity. In some embodiments, one or both of D326 and D510 are substituted with alanine.

In some embodiments, the engineered Cas protein is attached to, bound to, or fused with an effector domain, such as a transcriptional regulatory domain or an epigenetic modifying domain. In some embodiments, the effector domain is fused to the C-terminus of the engineered Cas protein. In some embodiments, the effector domain is fused to the N-terminus of the engineered Cas protein. In some embodiments, the effector domain comprises a subcellular localization signal. In some embodiments, the subcellular localization signals is an organelle localization signal, such as a nuclear localization signal (NLS), nuclear export signal (NES), or mitochondrial localization signal. In some embodiments, the effector domain comprises a polypeptide that can (i) cleave a nucleic acid (e.g., DNA and/or RNA), (ii) affect RNA stability, (iii) edit a nucleotide, (iv) activate transcription, (v) repress transcription, (iv) activate translation, (v) repress translation, (vi) methylate a nucleic acid (e.g., DNA and/or RNA), (vii) demethylate a nucleic acid (e.g., DNA and/or RNA), (viii) affect RNA splicing, (ix) enable affinity purification or immunoprecipitation (e.g., FLAG, HA, biotin, or HALO tags), and/or (x) enable proximity-based protein labeling and identification.

### II. Guide RNA

In another aspect, a single-guide RNA (sgRNA) protein is provided. The sgRNA disclosed herein provides surprising improvements in its ability to function in eukaryotic cells. The sgRNA protein has an engineered CRISPR RNA (crRNA)/trans-activating CRISPR RNA (tracrRNA) fusion nucleotide sequence that is at least 60% identical to a wild-type crRNA/tracrRNA fusion nucleotide sequence. The engineered crRNA/tracrRNA fusion nucleotide sequence can be, for example, at least 60%, at least 64%, at least 68%, at least 72%, at least 76%, at least 80%, at least 82%, at least 84%, at least 86%, at least 88%, at least 90%, at least 91%, at least 92%, at least 93%, at least 94%, at least 95%, at least 96%, at least 97%, at least 98%, or at least 99% identical to a wild-type tracrRNA nucleotide sequence. In terms of ranges, in some embodiments the engineered crRNA/tracRNA fusion nucleotide sequence is between 60% and 100% identical to a wild-type crRNA/tracRNA fusion nucleotide sequence, e.g., between 60% and 82%, between 63% and 86%, between 66% and 90%, between 70% and 95%, or between 74% and 100%. In some embodiments, the wild-type crRNA/tracrRNA fusion nucleotide sequence is included in the sequence of SEQ ID NO: 7.

In some embodiments, the engineered crRNA/tracrRNA fusion nucleotide sequence includes one or more modifications to the wild-type crRNA/tracrRNA fusion nucleotide sequence, where the modifications follow one or more particular rules determined to have surprising advantages for the characteristics of the provided sgRNA. For example, the particular modification rules have been selected for their ability to produce sgRNA capable of functioning within eukaryotic cells. According to these particular rules, all or some of the modifications to the wild-type crRNA/tracrRNA fusion nucleotide sequence involve either (1) substitution of at least one U of a poly-U region of the wild-type nucleotide sequence, e.g., with a G, (2) deletion of at least a portion of a 3′ region of the wild-type sequence corresponding to an RNA stem-loop hairpin structure, or (3) deletion of at least a portion of a 5′ poly-G region of the wild-type nucleotide sequence.

In some embodiments, the one or more modifications to the wild-type crRNA/tracrRNA fusion nucleotide sequence include modifications in one of the above three categories. The one or more modifications can include a modification in one category that is either category (1), (2), or (3). The one or more modifications can consist of a modification in one category that is either category (1), (2), or (3). In some embodiments, the one or more modifications include modifications in each of the two categories (1) and (2), (1) and (3), or (2) and (3). In some embodiments, the one or more modifications consist of modifications in each of the two categories (1) and (2), (1) and (3), or (2) and (3). In some embodiments, the one or more modifications include modifications in each of the three categories (1), (2) and (3).

In some embodiments, the wild-type crRNA/tracrRNA fusion nucleotide sequence includes a poly-U region proximate to the 3′ end of the sequence. The poly-U region can include, for example, four uracil nucleotides, five uracil nucleotides, six uracil nucleotide, seven uracil nucleotides, eight uracil nucleotides, or more than eight uracil nucleotides. In some embodiments, the engineered crRNA/tracrRNA fusion nucleotide sequence of the provided sgRNA includes substitution of at least one U of the poly-U region, where each substitution is independently to a G. The engineered crRNA/tracrRNA fusion nucleotide sequence can include substitution of only one uracil nucleotide of the poly-U region to a G, only two uracil nucleotides of the poly-U region each to a G, only thee uracil nucleotides of the poly-U region each to a G, only four uracil nucleotides of the poly-U region each to a G, only five uracil nucleotides of the poly-U region each to a G, only six uracil nucleotides of the poly-U region each to a G, only seven uracil nucleotides of the poly-U region each to a G, only eight uracil nucleotides of the poly-U region each to a G, or more than eight uracil nucleotides of the poly-U region each to a G. In some embodiments, each uracil nucleotide of the poly-U region is substituted with a G. In some embodiments, the substitutions in the poly-U region are contiguous. In some embodiments, the substitutions in the poly-U region are noncontiguous. In some embodiments, the engineered crRNA/tracrRNA fusion nucleotide sequence includes the sequence of SEQ ID NO: 8.

In some embodiments, the wild-type crRNA/tracrRNA fusion nucleotide sequence includes a 3′ region corresponding to an RNA stem-loop hairpin structure, and the engineered crRNA/tracrRNA fusion nucleotide sequence of the provided sgRNA includes deletion of at least a portion of the 3′ region. The amount of the 3′ wild-type crRNA/tracrRNA region corresponding to an RNA hairpin structure that is deleted in creating the engineered crRNA/tracrRNA fusion nucleotide sequence can be, for example, from 1% to 100%, e.g., from 1% to 60%, from 10% to 70%, from 20% to 80%, from 30% to 90%, or from 40% to 100%. In terms of upper limits, the amount of the 3′ wild-type crRNA/tracrRNA region corresponding to an RNA hairpin structure that is deleted in creating the engineered crRNA/tracrRNA fusion nucleotide sequence can be less than 100%, e.g., less than 90%, less than 80%, less than 70%, less than 60%, less than 50%, less than 40%, less than 30%, less than 20%, or less than 10%. In terms of lower limits, the amount of the 3′ wild-type crRNA/tracrRNA region corresponding to an RNA hairpin structure that is deleted in creating the engineered crRNA/tracrRNA fusion nucleotide sequence can be, for example, at least 1%, e.g., at least 10%, at least 20%, at least 30%, at least 40%, at least 50%, at least 60%, at least 70%, at least 80%, or at least 90%. In some embodiments, the only modification to the wild-type crRNA/tracrRNA fusion nucleotide sequence is deletion of at least a portion of the 3′ region corresponding to an RNA hairpin structure. In some embodiments, the engineered crRNA/tracrRNA nucleotide sequence includes the sequence of SEQ ID NO: 9.

In some embodiments, the wild-type crRNA/tracrRNA fusion nucleotide sequence includes a poly-G region proximate to the 5′ end of the sequence. The poly-G region can include, for example, three guanine nucleotides, four guanine nucleotides, five guanine nucleotide, six guanine nucleotides, seven guanine nucleotides, or more than seven uracil nucleotides. In some embodiments, the engineered crRNA/tracrRNA fusion nucleotide sequence of the provided sgRNA includes deletion of at least one guanine of the poly-G region. The engineered crRNA/tracrRNA fusion nucleotide sequence can include deletion of only one guanine nucleotide of the poly-G region, only two guanine nucleotides of the poly-G region, only thee guanine nucleotides of the poly-G region, only four guanine nucleotides of the poly-G region, only five guanine nucleotides of the poly-G region, only six guanine nucleotides of the poly-G region, only seven guanine nucleotides of the poly-G region, or more than seven guanine nucleotides of the poly-G region. In some embodiments, each guanine nucleotide of the poly-G region is deleted. In some embodiments, the engineered crRNA/tracrRNA fusion nucleotide sequence includes the sequence of SEQ ID NO: 10.

In some embodiments, the provided sgRNA includes a spacer nucleotide sequence corresponding to a 5′ Protospacer Adjacent Motif (PAM) having a 5′-TTTR-3′ nucleotide sequence. In some embodiments, the target nucleic acid associated with the provided sgRNA is dsDNA. In such embodiments, dsDNA-targeting specificity is determined, at least in part, by two parameters: the sgRNA spacer targeting a protospacer in the target dsDNA (the sequence in the target dsDNA corresponding to the sgRNA spacer on the non-complementary DNA strand) and a short PAM sequence located immediately 5′ (upstream) of the protospacer on the non-complementary DNA strand. In some embodiments, the PAM is 5′-TTTG-3′ or 5′-TTTA-3′. In some embodiments, the PAM is 5′-TTTG-3′. In some embodiments, the PAM is 5′-TTTA-3′.

### III. Nucleic Acids

In another aspect, a nucleic acid sequence is provided, where the nucleic acid sequence encodes any of the engineered Cas proteins disclosed herein and described in further detail above, or any of the sgRNA molecules disclosed herein and described in further detail above. In some embodiments, the nucleic acid sequence includes or consists of DNA. In some embodiments, the nucleic acid sequence includes or consists of RNA, e.g., mRNA.

### IV. Vectors

In another aspect, a vector is provided, where the vector includes at least one nucleic acid sequence encoding any of the engineered Cas proteins disclosed herein, or at least one nucleic acid sequence encoding any of the sgRNA molecules disclosed herein. In some embodiments, the provided vector includes at least one nucleic acid sequence encoding any of the engineered Cas proteins disclosed herein, and at least one nucleic acid sequence encoding any of the sgRNA molecules disclosed herein.

In some embodiments, the provided vector includes one or more nuclear localization signals (NLS). In some embodiments, the one or more nuclear localization signals include an SV40 NLS. In some embodiments, the one or more nuclear localization signals include a c-Myc NLS. In some embodiments, the one or more nuclear localization signals include both an SV40 NLS and a c-Myc NLS. In some embodiments, the one or more nuclear localization signals include two or more copies of an SV40 NLS. In some embodiments, the one or more nuclear localization signals include two or more copies of a c-Myc NLS. In some embodiments, the one or more nuclear localization signals include both one or more copies, e.g., two or more copies, of an SV40 NLS and one or more copies, e.g., two or more copies, of a c-Myc NLS.

In some embodiments, the provided vector includes one or more additional nucleic acid sequences that each independently encode a transcriptional activator. Transcriptional activators suitable for use with the provided vectors include, but are not limited to, a VP64 transcriptional activator, a tripartite VP64-p65-Rta (VPR) transcriptional activator, a p300 transcriptional activator, a TET1 transcriptional activator, a TET2 transcriptional activator, an HSF1 transcriptional activator, an NFAT transcriptional activator, an NFkB transcriptional activator, a PRDM transcriptional activator, and combinations thereof.

In some embodiments, the provided vector includes one or more additional nucleic acid sequences that each independently encode a transcriptional repressor. Transcriptional repressors suitable for use with the provided vectors include, but are not limited to, a KRAB transcriptional repressor, a bipartite KRAB-DNMT3L (KL) transcriptional repressor, a tripartite KRAB-DNMT3A-DNMT3L (KAL) transcriptional repressor, a SID transcriptional repressor, an HP1 transcriptional repressor, an EZH2 transcriptional repressor, and combinations thereof.

In some embodiments, the provided vector includes one or more sgRNAs configured to enable the simultaneous activation or repression of two or more reporter genes or endogenous genes.

In some embodiments, the provided vector further includes a base editor. In some embodiments, the provided vector further includes a prime editor. In some embodiments, the provided vector further includes a fluorescent protein. In some embodiments, the provided vector is a viral vector. The provided vector can be, for example, an adeno-associated virus (AAV) vector, an adenovirus vector, a retrovirus vector, a lentivirus vector, or a herpes simplex virus (HSV) vector.

### V. Systems

In another aspect, a system, e.g., a ribonucleotide complex, including a Cas protein and an sgRNA is provided. In some embodiments, the Cas protein of the provided system is any of the engineered Cas proteins disclosed herein and described in further detail above. In some embodiments, the sgRNA of the provided system is any of the sgRNA molecules disclosed herein and described in further detail above. In some embodiments, the provided system includes both one or more, e.g., two or more, Cas proteins disclosed herein, and one or more, e.g., two or more, sgRNA molecules disclosed herein.

In another aspect, a system including a nucleic acid encoding a Cas protein and a nucleic acid encoding an sgRNA is provided. In some embodiments, the nucleic acid encoding a Cas protein is any of those disclosed herein and described in further detail above. In some embodiments, the nucleic acid encoding an sgRNA is any of those disclosed herein and described in further detail above. In some embodiments, the provided system includes both one or more, e.g., two or more, nucleic acids each independently encoding a Cas protein as disclosed herein, and one or more, e.g., two or more, nucleic acids each independently encoding a sgRNA molecule as disclosed herein.

In general, the provided systems are characterized by elements that promote the formation of a nucleic acid-targeting complex including a Cas protein and an sgRNA at the site of a target sequence, which can be present in a DNA molecule or an RNA molecule. As used herein, the term “target sequence” refers to a sequence to which a guide sequence (also referred to herein as a “spacer” or “spacer sequence”) in an sgRNA is designed to have complementarity, where hybridization between the target sequence and the sgRNA allows for localization of the Cas protein to the target sequence. Full complementarity is not necessarily required, provided there is sufficient complementarity to allow for hybridization between the target sequence and the sgRNA. In some embodiments, a target sequence is located in the nucleus or cytoplasm of a cell. In some embodiments, the target sequence may be within an organelle of a eukaryotic cell, for example, a mitochondrion or a chloroplast.

In some embodiments, the target nucleotide sequence is present in a gene sequence. In some embodiments, the target nucleotide sequence is present in a promoter region of the gene, and can be present in the sense strand or anti-sense strand of the gene. In some embodiments, the target nucleotide sequence is present in a 5′ UTR region of the gene, and can be present in the sense strand or anti-sense strand of the gene. In some embodiments, the target nucleotide sequence is present in a 5′ UTR/RBS region of the gene, and can be present in the sense strand or anti-sense strand of the gene. In some embodiments, the target nucleotide sequence is present in a coding region of the gene, and can be present in the sense strand or anti-sense strand of the gene.

In some embodiments, one or more vectors driving expression of one or more elements of the provided system, and are introduced into a host cell such that expression of the elements of the system directs formation of a nucleic acid-targeting complex at one or more target sequence sites in the host cell. For example, a Cas protein and an sgRNA could each be operably linked to separate regulatory elements on separate vectors. Alternatively, two or more of the elements expressed from the same or different regulatory elements can be combined in a single vector, with one or more additional vectors providing any components of the system not included in the first vector. System elements that are combined in a single vector can be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction. In some embodiments, a single promoter drives expression of a transcript encoding a Cas protein and an sgRNA. In some embodiments, the Cas protein and sgRNA are operably linked to and expressed from the same promoter.

### VI. Pharmaceutical Compositions

In another aspect, a pharmaceutical composition is provided. The provided pharmaceutical composition includes one or more, e.g., two or more of any of the engineered Cas proteins as disclosed herein and described in further detail above; one or more, e.g., two or more, of any of the sgRNA molecules disclosed herein and described in further detail above; one or more, e.g., two or more, of any of the nucleic acids disclosed herein and described in further detail above; one or more, e.g., two or more, of any of the vectors disclosed herein and described in further detail above; one or more, e.g., two or more, of any of the systems disclosed herein and described in further detail above; or any combination thereof.

In some embodiments, the pharmaceutical composition includes a therapeutically effective amount of a pharmaceutically acceptable excipient. In some embodiments, the pharmaceutical composition includes one or more of a diluent, adjuvant, or carrier in a formulation suitable for administration, e.g., administration to a mammal. Suitable diluents, adjuvants, or carriers can include, for example, lipids, e.g., liposomes, e.g., liposome dendrimers; liquids, such as water and oils, including those of petroleum, animal, vegetable or synthetic origin, such as peanut oil, soybean oil, mineral oil, sesame oil and the like; gum acacia; gelatin; starch paste; talc; keratin; colloidal silica; urea; and the like. Additional examples of suitable diluents include distilled water, buffered water, physiological saline, PBS, Ringer's solution, dextrose solution, and Hank's solution. The pharmaceutical compositions can also include additional substances to approximate physiological conditions, such as pH adjusting and buffering agents, toxicity adjusting agents, wetting agents and detergents. In addition, auxiliary, thickening, lubricating and coloring agents can alternatively or additionally be used. Pharmaceutical compositions can be formulated into preparations in solid, semisolid, liquid or gaseous forms, such as tablets, capsules, powders, granules, ointments, solutions, suppositories, injections, inhalants, gels, microspheres, and aerosols.

The provided pharmaceutical composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and/or enhance solubility or uptake). Examples of such modifications or complexing agents include sulfate, gluconate, citrate, and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

### VII. Methods of Nucleic Acid Modulation

In another aspect, a method of modulating one or more target nucleic acids in a cell is provided. The method includes contacting the cell with one or more, e.g., two or more of any of the engineered Cas proteins as disclosed herein and described in further detail above; one or more, e.g., two or more, of any of the sgRNA molecules disclosed herein and described in further detail above; one or more, e.g., two or more, of any of the nucleic acids disclosed herein and described in further detail above; one or more, e.g., two or more, of any of the vectors disclosed herein and described in further detail above; one or more, e.g., two or more, of any of the systems disclosed herein and described in further detail above; or any combination thereof.

In some embodiments, the provided method results in selective modulation of one or more target nucleic acids in a cell. For example, selective modulation can modulate one or more target nucleic acids by at least 10%, e.g., at least 20%, at least 30%, at least 40%, at least 50%, at least 60%, at least 70%, at least 80%, at least 90%, or greater than 90%, compared to the level of modulation of the target nucleic acids in the absence of the contacting, while not substantially modulating any non-target nucleic acids. In some embodiments, selective modulation includes modulating any non-target nucleic acids by, less than 10%, e.g., less than 9%, less than 8%, less than 7%, less than 6%, less than 5%, less than 4%, less than 3%, less than 2%, or less than 1%.

In some embodiments, the provided method results in modulation of the transcription of one or more target nucleic acids. In some embodiments, the method results in the increase or decrease of transcription of target DNA. In some embodiments, the method is used to control the transcription of a targeted gene-coding RNA (protein-encoding mRNA) and/or a targeted non-coding RNA (e.g., tRNA, rRNA, snoRNA, siRNA, miRNA, long ncRNA. etc.). In some embodiments, the method modifies a polypeptide associated with DNA (e.g., histone). In some embodiments, method involves enzymatic activity such as methyltransferase activity, demethylase activity, acetyltransferase activity, deacetylase activity, kinase activity, phosphatase activity, ubiquitin ligase activity (e.g., ubiquitination activity), deubiquitinating activity, adenylation activity, deadenylation activity, SUMOylating activity, deSUMOylating activity, ribosylation activity, deribosylation activity, myristoylation activity, demyristoylation activity glycosylation activity (e.g., from GlcNAc transferase) or deglycosylation activity. The enzymatic activities listed herein catalyze covalent modifications to proteins. Such modifications are known in the art to alter the stability or activity of the target protein (e.g., phosphorylation due to kinase activity can stimulate or silence protein activity depending on the target protein). Of particular interest as protein targets are histones. Histone proteins are known in the art to bind DNA and form complexes known as nucleosomes. Histones can be modified (e.g., by methylation, acetylation, ubiquitination, phosphorylation) to elicit structural changes in the surrounding DNA, thus controlling the accessibility of potentially large portions of DNA to interacting factors such as transcription factors, polymerases and the like. A single histone can be modified in many different ways and in many different combinations (e.g., trimethylation of lysine 27 of histone 3, H3K27, is associated with DNA regions of repressed transcription while trimethylation of lysine 4 of histone 3, H3K4, is associated with DNA regions of active transcription).

In some embodiments, the provided method results in modulation of the translation of one or more target nucleic acids. In some embodiments, the method modulate translation of one or more target RNA molecules.

In some embodiments, the one or more target nucleic acids are in a cell that is a eukaryotic cell. In some embodiments, the one or more target nucleic acids are in a cell that is an animal cell. In some embodiments, the one or more target nucleic acids are in a cell that is a mammalian cell. In some embodiments, the one or more target nucleic acids are in a cell that is a human cell. In some embodiments, the one or more target nucleic acids are in a cell that is a stem cell. In some embodiments, the one or more target nucleic acids are in a cell that is a blood cell. In some embodiments, the one or more target nucleic acids are in a cell that is an immune cell. In some embodiments, the one or more target nucleic acids are in a cell that is a plant cell. In some embodiments, the one or more target nucleic acids are in a cell that is in vivo. In some embodiments, the one or more target nucleic acids are in a cell that is part of an organoid.

In some embodiments, the modulating of the one or more target nucleic acids includes activating at least one of the one or more target nucleic acids. In some embodiments, the modulating of the one or more target nucleic acids includes repressing at least one of the one or more target nucleic acids. In some embodiments, the modulating of the one or more target nucleic acids includes editing at least one of the one or more target nucleic acids. In some embodiments, the modulating of the one or more target nucleic acids includes editing a primer of at least one of the one or more target nucleic acids. In some embodiments, the modulating of the one or more target nucleic acids includes nicking at least one of the one or more target nucleic acids. In some embodiments, the modulating of the one or more target nucleic acids includes labeling at least one of the one or more target nucleic acids. In some embodiments, the modulating of the one or more target nucleic acids includes altering the spatiotemporal positioning of at least one of the one or more target nucleic acids. In some embodiments, the modulating of the one or more target nucleic acids includes altering the methylation of at least one of the one or more target nucleic acids. In some embodiments, the modulating of the one or more target nucleic acids includes altering the acetylation of at least one of the one or more target nucleic acids. In some embodiments, the modulating of the one or more target nucleic acids includes altering the acetylation of at least one histone or nucleosome associated with at least one of the one or more target nucleic acids. In some embodiments, the modulating of the one or more target nucleic acids includes altering the methylation of at least one histone or nucleosome associated with at least one of the one or more target nucleic acids.

### VIII. Methods of Treatment

In another aspect, a method of preventing a disorder, e.g., a genetic disorder, in a subject is provided. The method includes administering to the subject an amount of any of the pharmaceutical compositions disclosed herein and described in further detail above, where the administered amount is sufficient to modulate one or more target nucleic acids associated with the disorder. In some embodiments, the administered amount is sufficient to correct one or more mutations in the one or more target nucleic acids.

Disorders suitable for treating with the provided method include, but are not limited to, X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation disease or disorders, inflammation, facioscapulohumeral muscular dystrophy, retinitis pigmentosa, Leber congenital amaurosis, glaucoma, immune-related diseases or disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases and disorders.

Also provided is a method of treating an infection in a subject. The method includes administering to the subject an amount of any of the pharmaceutical compositions disclosed herein and described in further detail above, where the administered amount is sufficient to modulate one or more target nucleic acids associated with the infection. In some embodiments, the administered amount is sufficient to cut or nick at least one of the one or more target nucleic acids. In some embodiments, the infectious agent is a virus.

In some embodiments, the administering of any of the provided methods is via a delivery system using a virus, a nanoparticle, a liposome, a micelle, a virosome, a nucleic acid complex, a protein-RNA conjugate, and combinations thereof. Administration can be achieved in various ways, including oral, buccal, rectal, parenteral, intraperitoneal, intradermal, transdermal, intra-tracheal, intraocular, etc., administration. The active agent can be systemic after administration or can be localized by the use of regional administration, intramural administration, or use of an implant that acts to retain the active dose at the site of implantation. The active agent can be formulated for immediate activity or it may be formulated for sustained release.

In some embodiments, the administering of any of the provided methods is via a single adeno-associated virus delivery system. In some embodiments, the administering is via a dual adeno-associated virus delivery system.

As used herein, the term “subject” generally refers to a vertebrate, preferably a mammal, more preferably a human. In some cases, a subject is a patient. Mammals include, but are not limited to, murines, simians, humans, farm animals, sport animals, and pets. In some embodiments, the subject of any of the provided methods is human.

### IX. Embodiments

The following embodiments are contemplated. All combinations of features and embodiment are contemplated.

Embodiment 1: An engineered Clustered Regularly Interspaced Short Palindromic Repeat (CRISPR)-associated (Cas) protein that is functional in eukaryotic cells, the Cas protein comprising a modified amino acid sequence that is at least 80% identical to a native amino acid sequence of a wild-type Cas protein, wherein: the native amino acid sequence has a length of less than 700 amino acids and comprises a (D/E/K/N)X(R/F)(E/K)N (SEQ ID NO: 253) motif; and the modified amino acid sequence comprises one or more substitutions in the native amino acid sequence, wherein at least one of the one or more substitutions is at a position either (1) within or no more than 30 amino acids upstream or downstream of the (D/E/K/N)X(R/F)(E/K)N (SEQ ID NO: 253) motif, (2) at or no more than 30 amino acids upstream or downstream of position 241 of the native amino acid sequence, (3) at or no more than 30 amino acids upstream or downstream of position 516 of the native amino acid sequence, or (4) having an electrically charged amino acid in the native amino acid sequence.

Embodiment 2: An embodiment of embodiment 1, wherein at least one of the one or more substitutions is to an amino acid selected from the group consisting of arginine (R), alanine (A), serine(S), and glycine (G).

Embodiment 3: An embodiment of embodiment 1, wherein the wild-type Cas protein is a type V Cas protein.

Embodiment 4: An embodiment of embodiment 1, wherein the wild-type Cas protein is a wild-type V-F (Cas14 or Cas 12f) protein or a wild-type V-J (Cas@ or Cas 12J) protein.

Embodiment 5: An embodiment of embodiment 1, wherein the native amino acid sequence is the sequence of SEQ ID NO: 1.

Embodiment 6: An embodiment of any of the embodiments of embodiment 1-5,wherein the one or more substitutions comprise a substitution at a position selected from the group consisting of D143, K11, K73, T147, E151, K154, E241, D318, K330, K457, E425, E462, N504, E507, N516, N519, E527, and E528.

Embodiment 7: An embodiment of embodiment 6, wherein the substitution is selected from the group consisting of D143R, K11R, K73R, T147R, E151R, K154R, E241R, D318R, K330R, E425N, K457R, E462R, N504R, E507R, N516R, N519R, E527R, and E528R.

Embodiment 8: An embodiment of embodiment 7, wherein the substitution is selected from the group consisting of D143R, T147R, E151R, and E241R.

Embodiment 9: An embodiment of any of the embodiments of embodiment 6-8, wherein the one or more substitutions comprise two substitutions at positions selected from the group consisting of D143, T147, E151, K154, E241, K330, E425, N504, E507, N516, N519, E527, and E528.

Embodiment 10: An embodiment of embodiment 9, wherein the two substitutions comprise a substitution at D143 and a substitution at a position selected from the group consisting of T147, E151, K154, E241, K330R, E425N, N504, E507, N516, N519, E527, and E528.

Embodiment 11: An embodiment of embodiment 9 or 10, wherein the two substitutions are selected from the group consisting of D143R, T147R, E151R, E151A, K154R, E241R, N504R, E507R, N516R, N519R, E527R, and E528R.

Embodiment 12: An embodiment of any of the embodiments of embodiment 11, wherein the two substitutions are selected from the group consisting of D143R/T147R, D143R/E151R, D143R/E241R, D143R/E425N, D143R/E507R, D143R/N519R, D143R/E527R, D143R/E528R, D143R/R151S, D143/R151G, and D143R/E151A.

Embodiment 13: An embodiment of any of the embodiments of embodiment 9-12, wherein the one or more substitutions comprise three substitutions at positions selected from the group consisting of D143, T147, E151, K154, E241, K330, E425, N504, E507, N516, N519, E527, and E528.

Embodiment 14: An embodiment of embodiment 13, wherein the three substitutions comprise substitutions at D143 and T147 and a substitution at a position selected from the group consisting of E151, K154, E241, K330, E425, N504, E507, N516, N519, E527, and E528.

Embodiment 15: An embodiment of embodiment 13 or 14, wherein the three substitutions are selected from the group consisting of D143R, T147R, E151R, E151A, E151S, E151G, K154R, E241R, K330R, E425N, N504R, E507R, N516R, N519R, E527R, and E528R.

Embodiment 16: An embodiment of embodiment 15, wherein the three substitutions are selected from the group consisting of D143R/T147R/K330R, D143R/T147R/K154R, D143R/T147R/E241R, D143R/T147R/E507R, D143R/T147R/N519R, D143R/T147R/E527R, D143R/T147R/E528R, D143R/T147R/E151S, D143R/T147R/E151G, and D143R/T147R/E151A.

Embodiment 17: An embodiment of any of the embodiments of embodiment 13-16, wherein the one or more substitutions comprise four substitutions at positions selected from the group consisting of D143, T147, E151, K154, E241, K330, E425, N504, E507, N516, N519, E527, and E528.

Embodiment 18: An embodiment of embodiment 17, wherein the four substitutions comprise substitutions at D143, T147, and K330 and a substitution at a position selected from the group consisting of E151, K154, E241, E425, N504, E507, N516, N519, E527, and E528.

Embodiment 19: An embodiment of embodiment 17 or 18, wherein the four substitutions are selected from the group consisting of D143R, T147R, E151R, E151A, E151S, E151G, K154R, E241R, K330R, E425N, N504R, E507R, N516R, N519R, E527R, and E528R.

Embodiment 20: An embodiment of embodiment 19, wherein the four substitutions are selected from the group consisting of D143R/T147R/K330R/E528R, D143R/T147R/K330R/E151A, and D143R/T147R/K330R/E527R.

Embodiment 21: An embodiment of any of the embodiments of embodiment 17-20, wherein the one or more substitutions comprise five substitutions at positions selected from the group consisting of D143, T147, E151, K154, E241, K330, N504, E507, N516, N519, E527, and E528.

Embodiment 22: An embodiment of embodiment 21, wherein the five substitutions comprise substitutions at D143, T147, K330, E151, and a substitution at a position selected from the group consisting of. K154, E241, N504, E507, N516, N519, E527, and E528.

Embodiment 23: An embodiment of embodiment 21 or 22, wherein the five substitutions are selected from the group consisting of D143R, T147R, E151R, E151A, K154R, E241R, K330R, N504R, E507R, N516R, N519R, E527R, and E528R.

Embodiment 24: An embodiment of embodiment 23, wherein the five substitutions are selected from the group consisting of D143R/T147R/K330R/E151A/E528R and D143R/T147R/K330R/E151A/E527R.

Embodiment 25: An embodiment of any of the embodiments of embodiment 1-24, wherein the Cas protein is a fully or partially nuclease deactivated Cas (dCas) protein.

Embodiment 26: An embodiment of embodiment 1, wherein the modified amino acid sequence comprises a sequence selected from the group consisting of SEQ ID NOS: 2, 3, 4, 5, and 6.

Embodiment 27: A single-guide RNA (sgRNA) comprising an engineered CRISPR RNA (crRNA)/trans-activating CRISPR RNA (tracrRNA) fusion nucleotide sequence that is at least 60% identical to a wild-type crRNA/tracrRNA fusion nucleotide sequence, wherein: the wild-type crRNA/tracrRNA fusion nucleotide sequence comprises (1) a 3′ region corresponding to an RNA stem-loop hairpin structure, (2) a poly-U region proximate to the 3′ region, and (3) a 5′ poly-G region; and the engineered crRNA/tracrRNA fusion nucleotide sequence comprises one or more modifications to the wild-type crRNA/tracrRNA fusion nucleotide sequence, the modifications selected from the group consisting of: substitution of at least one U of the poly-U region, deletion of at least a portion of the 3′ region; and deletion of at least a portion of the 5′ poly-G region.

Embodiment 28: An embodiment of embodiment 27, wherein the wild-type crRNA/tracrRNA fusion nucleotide sequence is the sequence of SEQ ID NO: 7.

Embodiment 29: An embodiment of embodiment 27 or 28, wherein the engineered crRNA/tracrRNA fusion nucleotide sequence comprises substitutions of at least one U of the poly-U region with a G.

Embodiment 30: An embodiment of embodiment 29, wherein the engineered crRNA/tracrRNA fusion nucleotide sequence comprises the sequence of SEQ ID NO: 8.

Embodiment 31: An embodiment of embodiment 29, wherein the engineered crRNA/tracrRNA fusion nucleotide sequence comprises deletion of at least a portion of the 3′ region.

Embodiment 32: An embodiment of embodiment 31, wherein the engineered crRNA/tracrRNA fusion nucleotide sequence comprises the sequence of SEQ ID NO: 9.

Embodiment 33: An embodiment of embodiment 31, wherein the engineered crRNA/tracrRNA fusion nucleotide sequence comprises deletion of at least a portion of the 5′ poly-G region.

Embodiment 34: An embodiment of embodiment 33, wherein the engineered crRNA/tracrRNA fusion nucleotide sequence comprises the sequence of SEQ ID NO: 10.

Embodiment 35: An embodiment of any of the embodiments of embodiment 27-34, further comprising a spacer nucleotide sequence corresponding to a 5′ Protospacer Adjacent Motif (PAM) having a 5′-TTTR-3′ nucleotide sequence.

Embodiment 36: A nucleic acid sequence encoding the engineered Cas protein of any of the embodiments of embodiment 1-26.

Embodiment 37: A nucleic acid sequence encoding the sgRNA of any of the embodiments of embodiment 27-35.

Embodiment 38: A vector comprising one or both of the nucleic acid of embodiment 36 and the nucleic acid of embodiment 37.

Embodiment 39: An embodiment of embodiment 38, further comprising one or more nuclear localization signals (NLS).

Embodiment 40: An embodiment of embodiment 39, wherein the one or more nuclear localization signals comprise one or both of an SV40 NLS and a c-Myc NLS.

Embodiment 41: An embodiment of any of the embodiments of embodiment 38-40, further comprising one or more additional nucleic acid sequences each independently encoding a transcriptional or epigenetically modifying activator.

Embodiment 42: An embodiment of embodiment 41, wherein the one or more transcriptional or epigenetically modifying activators comprise one or more of a VP64 transcriptional activator, a tripartite VP64-p65-Rta (VPR) transcriptional activator, a p300 transcriptional activator, a TET1 transcriptional activator, a TET2 transcriptional activator, an NFAT transcriptional activator, an NFkB transcriptional activator, and a PRDM transcriptional activator.

Embodiment 43: An embodiment of any of the embodiments of embodiment 38-42, further comprising one or more additional nucleic acid sequences each independently encoding a transcriptional or epigenetically modifying repressor.

Embodiment 44: An embodiment of embodiment 43, wherein the one or more transcriptional or epigenetically modifying repressors comprise one or more of a KRAB transcriptional repressor, a DNMT3A DNA methyltransferase, a DNMT3B DNA methyltransferase, a DNMT3L DNA methyltransferase, a bipartite KRAB-DNMT3A (KA) repressor, a bipartite KRAB-DNMT3L (KL) repressor, a tripartite KRAB-DNMT3A-DNMT3L (KAL) repressor, an SID transcriptional repressor, an EZH2 transcriptional repressor, an HP1 heterochromatin protein, a Gli3 transcriptional repressor, and a MBD3 transcriptional repressor.

Embodiment 45: An embodiment of any of the embodiments of embodiment 38-44, further comprising one or more additional nucleic acid sequences each independently encoding a base editor.

Embodiment 46: An embodiment of any of the embodiments of embodiment 38-45, further comprising one or more additional nucleic acid sequences each independently encoding a prime editor.

Embodiment 47: An embodiment of any of the embodiments of embodiment 38-46, further comprising one or more additional nucleic acid sequences each independently encoding a fluorescent protein.

Embodiment 48: An embodiment of any of the embodiments of embodiment 38-47, further comprising one or more additional nucleic acid sequences each independently encoding a retron element.

Embodiment 49: An embodiment of any of the embodiments of embodiment 38-48, wherein the vector is an adeno-associated virus (AAV) vector, an adenovirus vector, a retrovirus vector, a lentivirus vector, or a herpes simplex virus (HSV) vector.

Embodiment 50: A system comprising: the engineered Cas protein of any of the embodiments of embodiment 1-26; and an sgRNA.

Embodiment 51: A system comprising: a Cas protein; and the sgRNA of any of the embodiments of embodiment 27-35.

Embodiment 52: An embodiment of embodiment 51, wherein the Cas protein is the engineered Cas protein of any of the embodiments of embodiment 1-26.

Embodiment 53: A system comprising: the nucleic acid sequence of embodiment 36; and a nucleic acid sequence encoding an sgRNA.

Embodiment 54: A system comprising: a nucleic acid sequence encoding a Cas protein; and the nucleic acid sequence of embodiment 37.

Embodiment 55: An embodiment of embodiment 54, wherein the nucleic acid encoding a Cas protein is the nucleic acid sequence of embodiment 36.

Embodiment 56: A method of modulating one or more target nucleic acids in a cell, the method comprising contacting the cell with the engineered Cas protein of any of the embodiments of embodiment 1-26, the sgRNA of any of the embodiments of embodiment 27-35, the nucleic acid of any of the embodiments of embodiment 36 or 37, the vector any of the embodiments of embodiment 38-49, or the system of any of the embodiments of embodiment 50-55.

Embodiment 57: An embodiment of embodiment 56, wherein the cell is a eukaryotic cell.

Embodiment 58: An embodiment of embodiment 57, wherein the eukaryotic cell is an animal cell.

Embodiment 59: An embodiment of embodiment 58, wherein the animal cell is a mammalian cell.

Embodiment 60: An embodiment of embodiment 59, wherein the mammalian cell is a human cell.

Embodiment 61: An embodiment of any of the embodiments of embodiment 57-60, wherein the cell is a stem cell.

Embodiment 62: An embodiment of any of the embodiments of embodiment 57-61, wherein the cell is a blood cell.

Embodiment 63: An embodiment of any of the embodiments of embodiment 57-62, wherein the cell is an immune cell.

Embodiment 64: An embodiment of embodiment 57, wherein the cell is a plant cell.

Embodiment 65: An embodiment of any of the embodiments of embodiment 56-64, wherein the cell is in vivo.

Embodiment 66: An embodiment of any of the embodiments of embodiment 56-64, wherein the cell is part of an organoid.

Embodiment 67: An embodiment of any of the embodiments of embodiment 56-66, wherein the modulating comprises activating at least one of the one or more target nucleic acids.

Embodiment 68: An embodiment of any of the embodiments of embodiment 56-67, wherein the modulating comprises repressing at least one of the one or more target nucleic acids.

Embodiment 69: An embodiment of any of the embodiments of embodiment 56-68, wherein the modulating comprises editing at least one of the one or more target nucleic acids.

Embodiment 70: An embodiment of any of the embodiments of embodiment 56-69, wherein the modulating comprises editing a primer of at least one of the one or more target nucleic acids.

Embodiment 71: An embodiment of embodiment 69 or 70, wherein the editing comprises nicking the at least one target nucleic acid.

Embodiment 72: An embodiment of embodiment 69 or 70, wherein the editing comprises performing one or more gene knockouts.

Embodiment 73: An embodiment of embodiment 69 or 70, wherein the editing comprises performing one or more gene knock-ins.

Embodiment 74: An embodiment of embodiment 69 or 70, wherein the editing comprises performing one or more base substitutions.

Embodiment 75: An embodiment of any of the embodiments of embodiment 56-74, wherein the modulating comprises cutting genomic DNA.

Embodiment 76: An embodiment of embodiment 75, wherein the modulating further comprises mutating the genomic DNA.

Embodiment 77: An embodiment of embodiment 76, wherein the mutating comprises one or more insertions, one or more deletions, one or more substitutions, or a combination thereof.

Embodiment 78: An embodiment of any of the embodiments of embodiment 56-77, wherein the modulating comprises labeling at least one of the one or more target nucleic acids.

Embodiment 79: An embodiment of any of the embodiments of embodiment 56-78, wherein the modulating comprises altering the spatiotemporal positioning of at least one of the one or more target nucleic acids within the cell.

Embodiment 80: An embodiment of any of the embodiments of embodiment 56-79, wherein the modulating comprises altering the methylation of at least one of the one or more target nucleic acids.

Embodiment 81: An embodiment of any of the embodiments of embodiment 56-80, wherein the modulating comprises altering the acetylation of at least one of the one or more target nucleic acids.

Embodiment 82: An embodiment of any of the embodiments of embodiment 56-81, wherein the modulating comprises altering the acetylation of at least one histone or nucleosome associated with at least one of the one or more target nucleic acids.

Embodiment 83: An embodiment of any of the embodiments of embodiment 56-82, wherein the modulating comprises altering the methylation of at least one histone or nucleosome associated with at least one of the one or more target nucleic acids.

Embodiment 84: A pharmaceutical composition comprising the engineered Cas protein of any of the embodiments of embodiment 1-26, the sgRNA of any of the embodiments of embodiment 27-35, the nucleic acid of embodiment 36 or 37, the vector of any of the embodiments of embodiment 38-49, or the system of any of the embodiments of embodiment 50-55.

Embodiment 85: An embodiment of embodiment 84, further comprising a therapeutically effective amount of a pharmaceutically acceptable excipient.

Embodiment 86: A method of preventing or treating a genetic disorder in a subject, the method comprising administering to the subject an amount of the pharmaceutical composition of embodiment 84 or 85, wherein the amount is sufficient to modulate one or more target nucleic acids associated with the genetic disorder.

Embodiment 87: An embodiment of embodiment 86, wherein the amount is sufficient to correct one or more mutations in the one or more target nucleic acids.

Embodiment 88: An embodiment of embodiment 86 or 87, wherein the genetic disorder is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation disease or disorders, inflammation, facioscapulohumeral muscular dystrophy, retinitis pigmentosa, Leber congenital amaurosis, glaucoma, immune-related diseases or disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases and disorders.

Embodiment 89: An embodiment of any of the embodiments of embodiment 86-88, wherein the administering is via a delivery system selected from the group consisting of a virus, a nanoparticle, a liposome, a micelle, a virosome, a nucleic acid complex, a protein-RNA conjugate, and combinations thereof.

Embodiment 90: An embodiment of embodiment 89, wherein the administering is via a single adeno-associated virus delivery system.

Embodiment 91: An embodiment of embodiment 89, wherein the administering is via a dual adeno-associated virus delivery system.

Embodiment 92: An embodiment of any of the embodiments of embodiment 86-91, wherein the subject is human.

Embodiment 93: A method of treating an infection in a subject, the method comprising administering to the subject an amount of the pharmaceutical composition of embodiment 84 or 85, wherein the amount is sufficient to modulate one or more target nucleic acids associated with the infection.

Embodiment 94: An embodiment of embodiment 93, wherein the one or more target nucleic acids are nucleic acids of an infectious agent causing the infection.

Embodiment 95: An embodiment of embodiment 94, wherein the amount is sufficient to cut or nick at least one of the one or more target nucleic acids.

Embodiment 96: An embodiment of embodiment 94 or 95, wherein the infectious agent is a virus.

Embodiment 97: An embodiment of any of the embodiments of embodiment 93-96, wherein the administering is via a delivery system selected from the group consisting of a virus, a nanoparticle, a liposome, a micelle, a virosome, a nucleic acid complex, a protein-RNA conjugate, and combinations thereof.

Embodiment 98: An embodiment of embodiment 97, wherein the administering is via a single adeno-associated virus delivery system.

Embodiment 99: An embodiment of embodiment 97, wherein the administering is via a dual adeno-associated virus delivery system.

Embodiment 100: An embodiment of any of the embodiments of embodiment 93-99, wherein the subject is human.

### X. Examples

The present disclosure will be better understood in view of the following non-limiting examples. The following examples are intended for illustrative purposes only and do not limit in any way the scope of the present invention.

### Example 1. Functional Testing of Cas14 in Mammalian Cells

To test whether the naturally occurring Cas14 can function in mammalian cells, nuclease deactivated Cas14 was generated by introducing two mutations to the conserved active sites of Cas14 in the RuvC domains (L. B. Harrington et al., Science 362, (2018): 839-42; T. Karvelis et al., Nucleic Acids Res. 48, (2020): 5016-23). Specifically, the Cas14 sequence was amplified from plasmid Addgene #112500, and the dCas14 was generated by introducing two mutations (D326A and D510A) to the wild-type sequence. The resulting protein was then fused to a tripartite VPR activator (A. Chavez et al. Cell 155, (2016): 563-67) (FIG. 2). This Cas construct and others described in these Examples were cloned using InFusion and Stellar competent cells (Takara Bio).

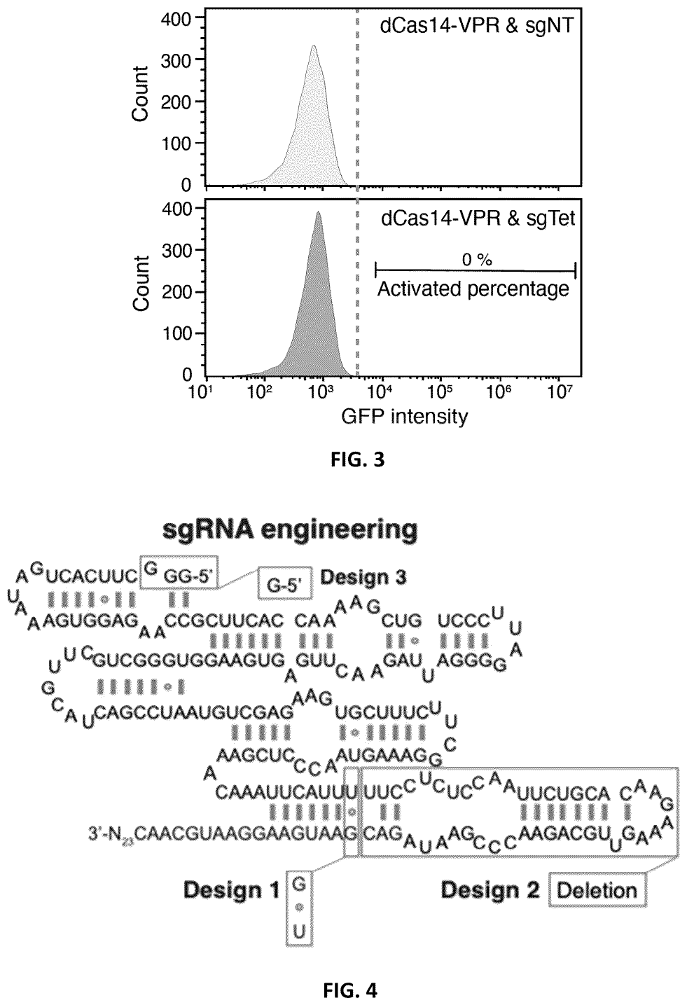

Using a doxycycline (Dox)-inducible TRE3G-EGFP HEK293T reporter cell line and a sgRNA targeting the promoter, activation efficiency of dCas14-VPR was measured. Wild-type HEK293T cells (ATCC) and the HEK293T TRE3G-GFP reporter line stably encoding EGFP under a Tet-On promoter (pTRE3G) (Y. Gao et al., Genome Res. 22, (2012): 1760-74) were cultured in DMEM with high glucose, sodium pyruvate and GlutaMAX (Thermo Fisher), additionally supplemented with 10% FBS (Sigma). Cells were grown at 37° C. and 5% CO₂ and maintained at confluency below 80%. All transfections were performed with TransIT-LTI transfection reagent (Mirus) at a ratio of 3 μL reagent per μg of plasmid. Cells were plated 1 day before at 1×10⁵ cells/mL. For this and other GFP activation assays described in these Examples, 500 ng of dCas constructs and 250 ng sgRNA or crRNA plasmids were transfected to HEK293T TRE3G-GFP cells in 24-well plates. Transfected cells were analyzed 2 days post-transfection for GFP activation. Here, no reporter activation was observed (FIG. 3), implying the natural Cas14 system fails to function in the human genome context.

### Example 2. Cas14 Guide RNA Engineering

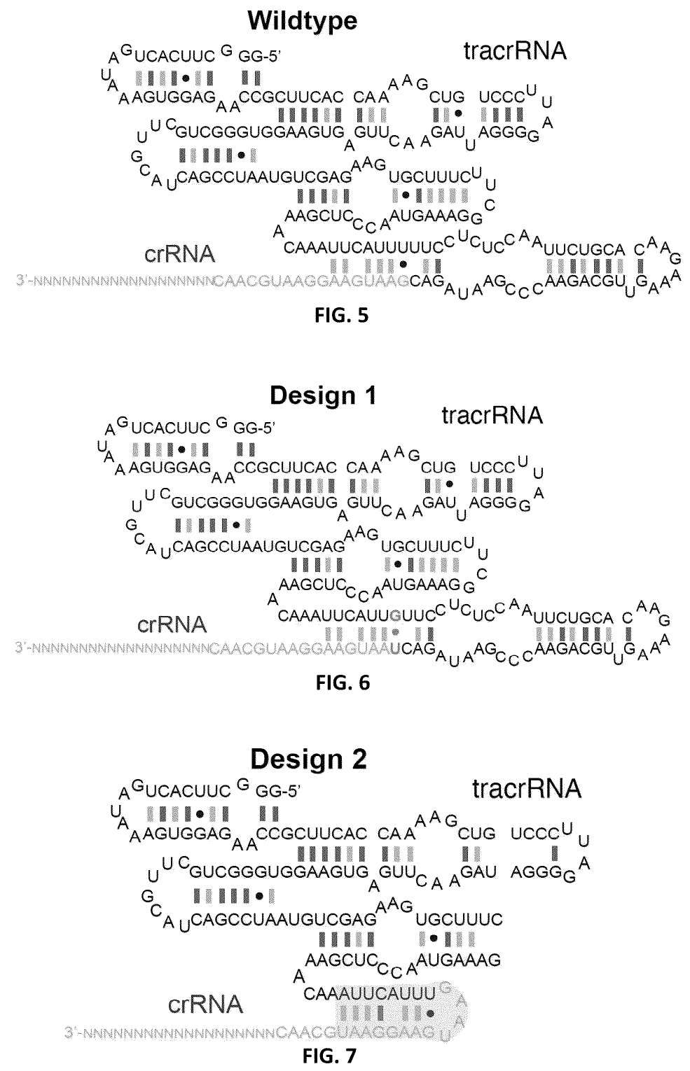

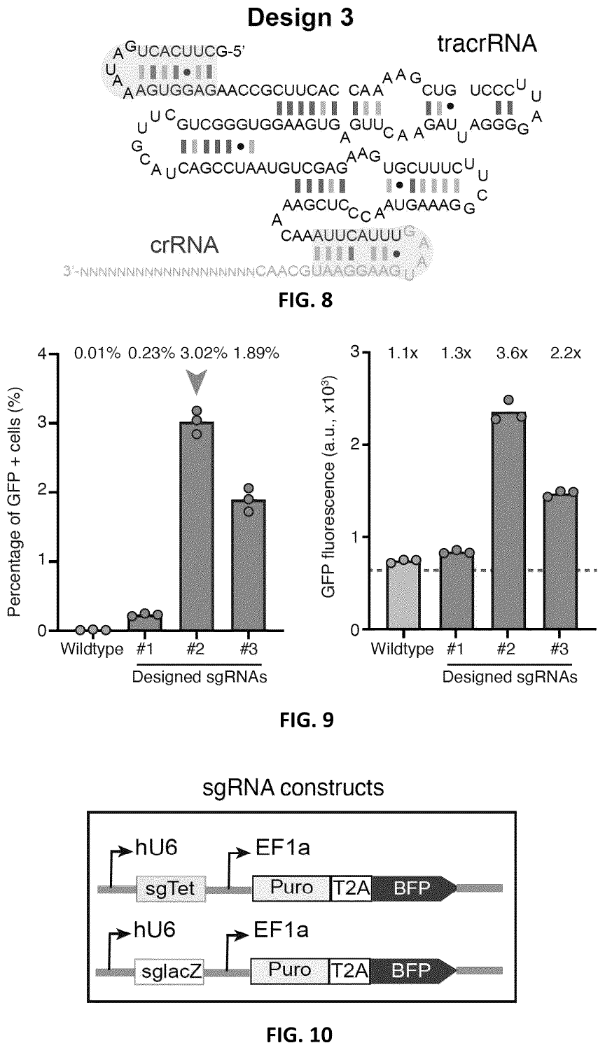

To test if the lack of Cas14 activity observed in Example 1 could be attributed to suboptimized design of sgRNA and/or the weak binding activity of Cas14 to the chromatin DNA, alternate guide RNA designs were tested (FIG. 4). These alternate designs were based on the natural tracrRNA sequence (FIG. 5), and included a G-U swap to disrupt the poly U sequence (Design 1 in FIG. 4, FIG. 6), RNA hairpin truncation (Design 2 in FIG. 4, FIG. 7), and poly G removal (Design 3 in FIG. 4, FIG. 8) (B. Chen et al., Cell 155, (2013): 1479-91).

The sgRNA backbone fragments were ordered via gBlocks from Integrated DNA Technologies (IDT). The sgRNA and/or crRNA plasmids described in this and other Examples were cloned using T4 DNA Ligase (New England Biolabs). To analyze fluorescent protein expression, cells were dissociated using 0.05% Trypsin EDTA (Life Technologies), resuspended in PBS with 5% FBS, and analyzed by flow cytometry on CytoFLEX S flow cytometer (Beckman Coulter). At least 10,000 cells containing constructs of interest of each sample were analyzed using FlowJo. The analyzed cells were gated for positive fluorescent protein expression based on the non-transfected control corresponding to construct expression.

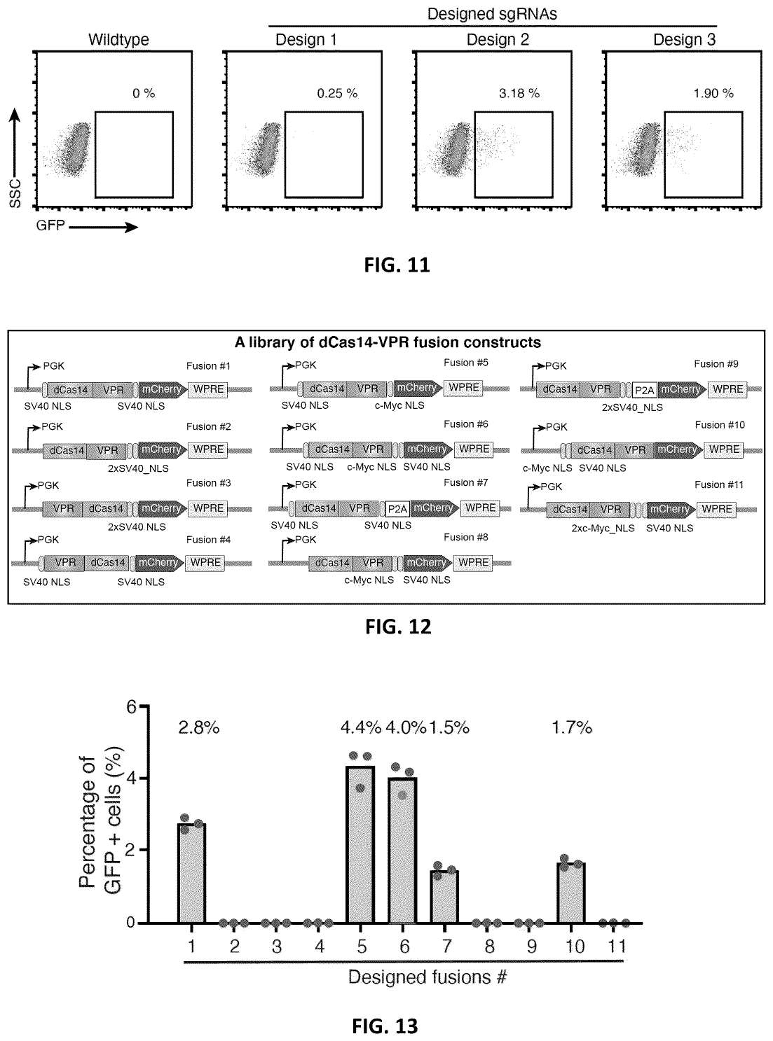

Interestingly, each of the tested sgRNA designs improved gene activation, with the Design 2 outperforming the others. While the wild-type sgRNA showed no activation, Design 2 sgRNA exhibited modest activation (3% of GFP+cells and 3.6-fold upregulation over the non-targeting sgRNA) (FIGS. 9-11). Design 2 was used for later Examples.

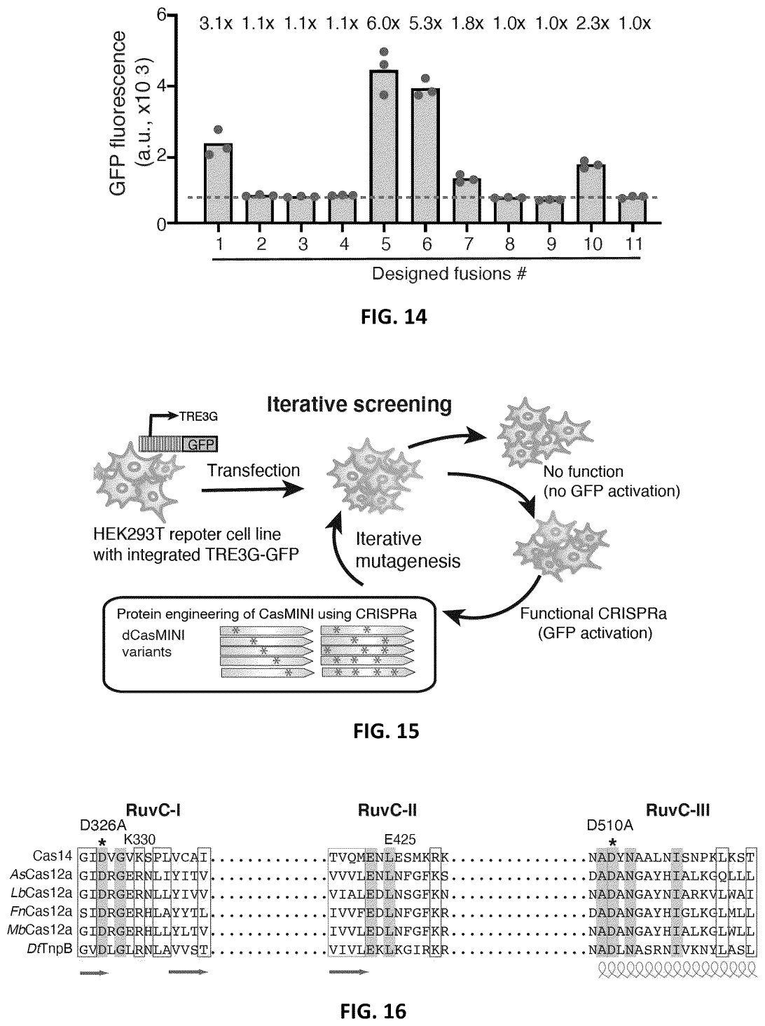

A library of fusions with different protein fusions, linkers, and nuclear localization signals was also tested. (FIG. 12). Using the sgRNA Design 2, we found one protein fusion outperformed other fusions (FIGS. 13 and 14), and was used for later Examples. Despite this optimization, however, other fusions showed only modestly lower reporter activation.

### Example 3. Cas14 Protein Engineering

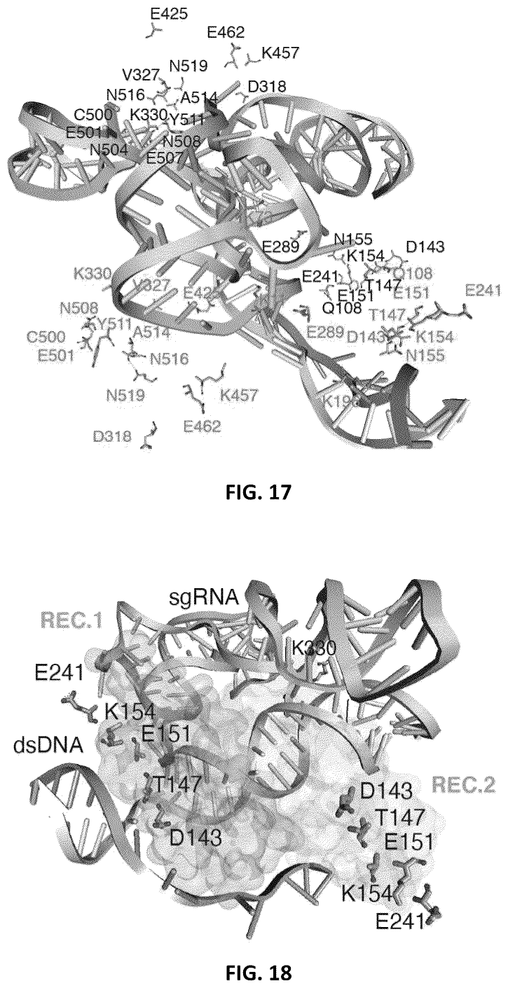

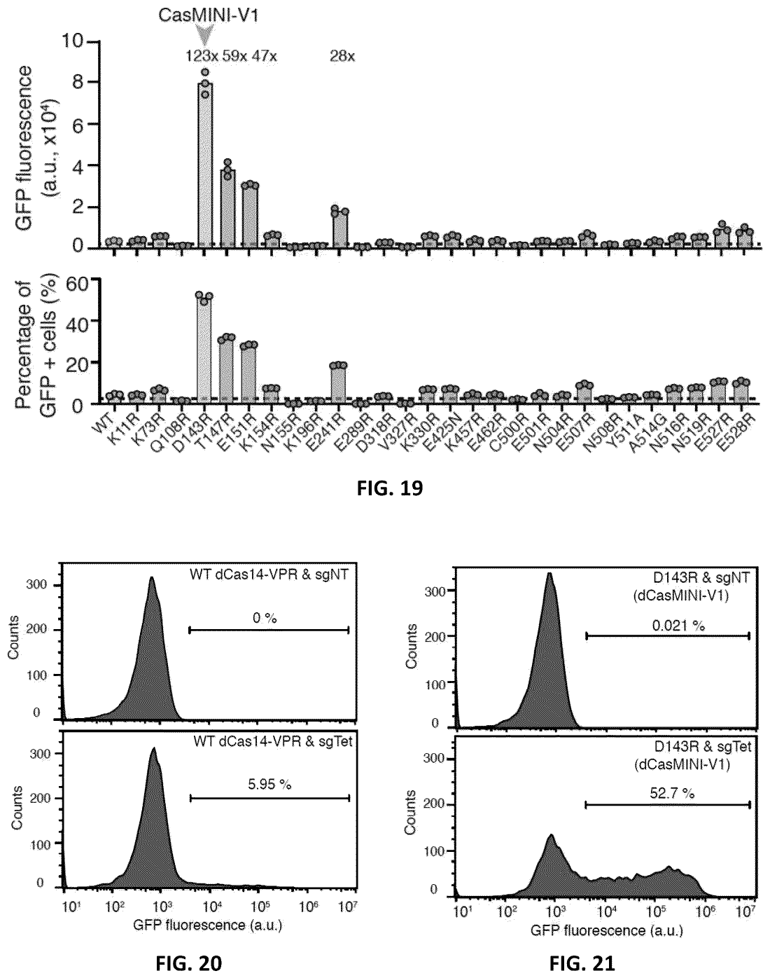

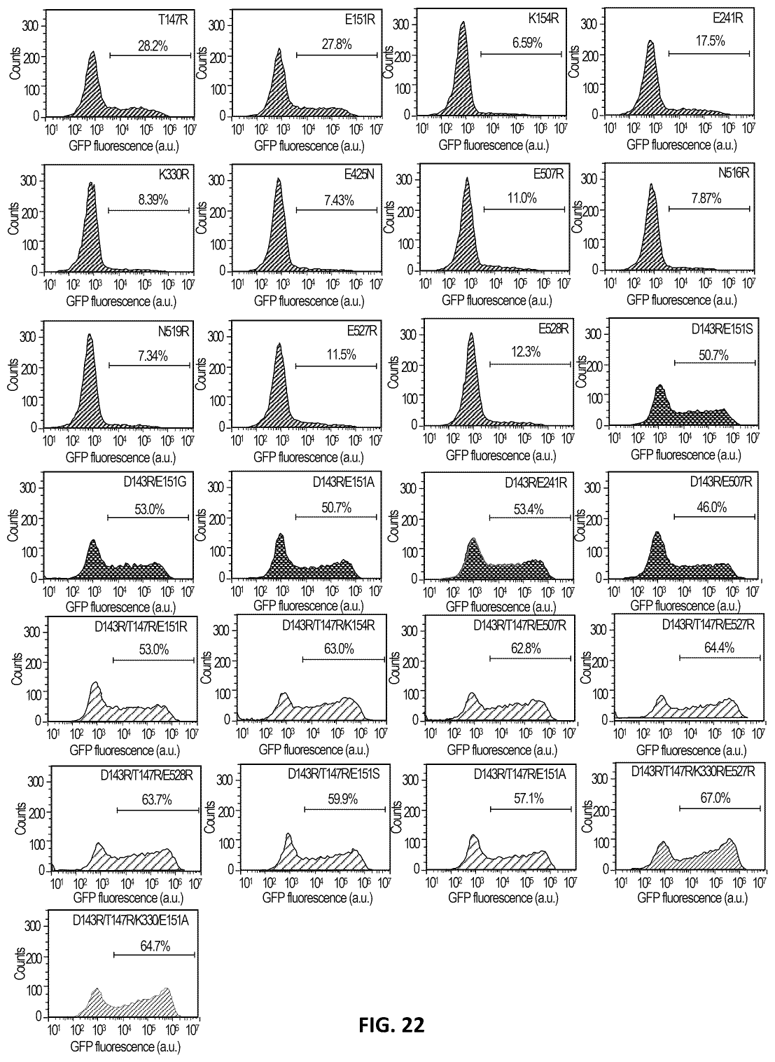

A guided iterative protein engineering strategy was next used to improve the performance of Cas14 variants as screened via a gene activation assay (FIG. 15). In this assay, variants showing enhanced activation of GFP in each cycle were used as the starting point of the next cycle. For initial mutations, the protein sequences of Cas14 was aligned to a family of Cas12a proteins for identification of predicted conserved motifs and residues in the target DNA binding pocket (FIGS. 16-18). Based on this analysis, 28 variants were engineered by substituting each identified and targeted amino acid to the positively charged arginine (R) that might enhance Cas14-DNA interaction, or by substituting selected individual residues to the conserved residues in Cas12a (FIG. 19). Surprisingly, while most variants showed no improvement of activation over the wild-type dCas14-VPR, a few variants (D143R, T147R, E151R, E241R) significantly enhanced activation (FIGS. 19 and 22). D143R (CasMINI-V1) in particular showed more than 24-fold of improvement over the wild-type (FIGS. 19-21).

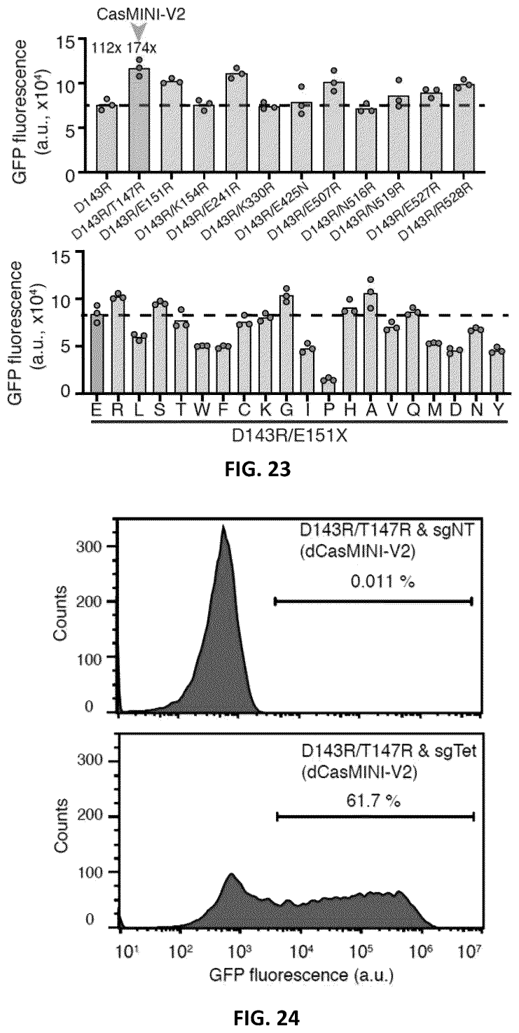

Two libraries with double mutations were generated for the second round of screening: one had 11 variants all containing D143R, and the other had 20 variants all containing D143R and a saturation mutation of E151 to all 19 non-glutamic acid (E) amino acids. From the first library, D143R/T147R, D143R/E151R, D143R/E241R, D143R/E507R were shown to exhibit improvement over the D143R variant. The D143R/T147R variant (CasMINI-V2) showed the best improvement (1.55-fold improvement over the best single variant) (FIG. 23). From the second library, in addition to substitutions with R, those with serine (S), glycine (G), or alanine (A) also improved activation (FIG. 23). These results suggest that replacement with small-size amino acids is important for enhanced activity.

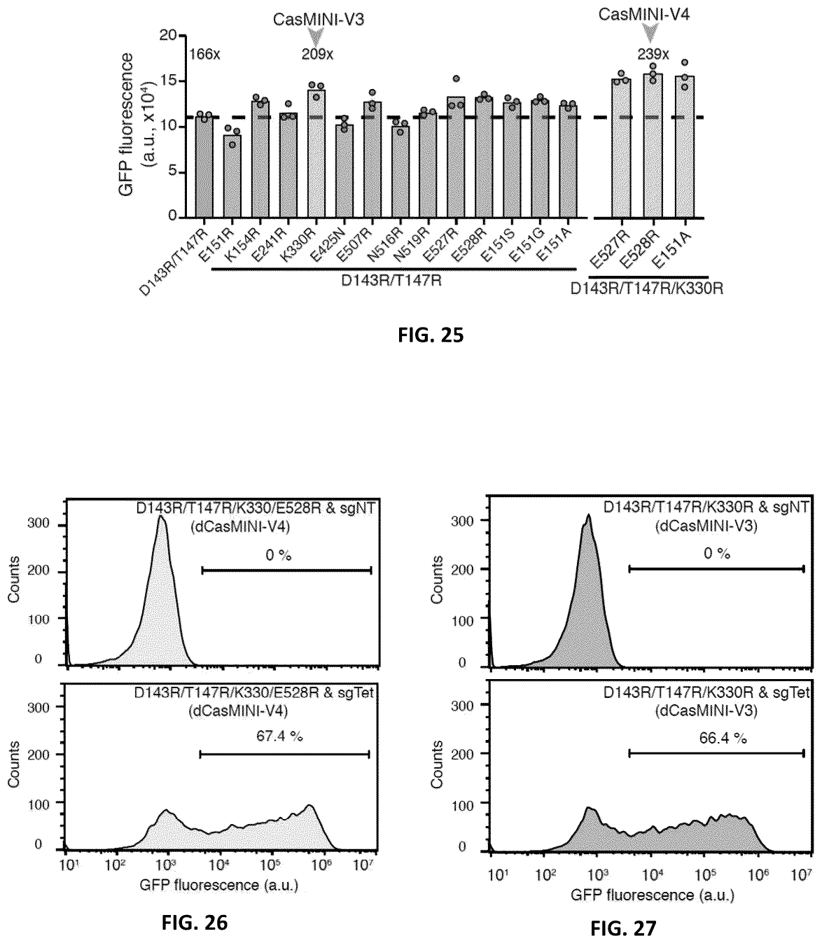

The third screening round of screen included 13 triple variants based on D143R/T147R. The D143R/T147R/K330R variant (CasMINI-V3) outperformed other variants (1.26-fold over the best double variant D143R/T147R, FIG. 25). The fourth round of screen testing a quadruple library based on D143R/T147R/K330R showed one variant D143R/T147R/K330R/E528R (CasMINI-V4) with 1.15-fold improvement over the best triple variant (FIG. 25). This variant was chosen as ‘CasMINI’ for further characterization.

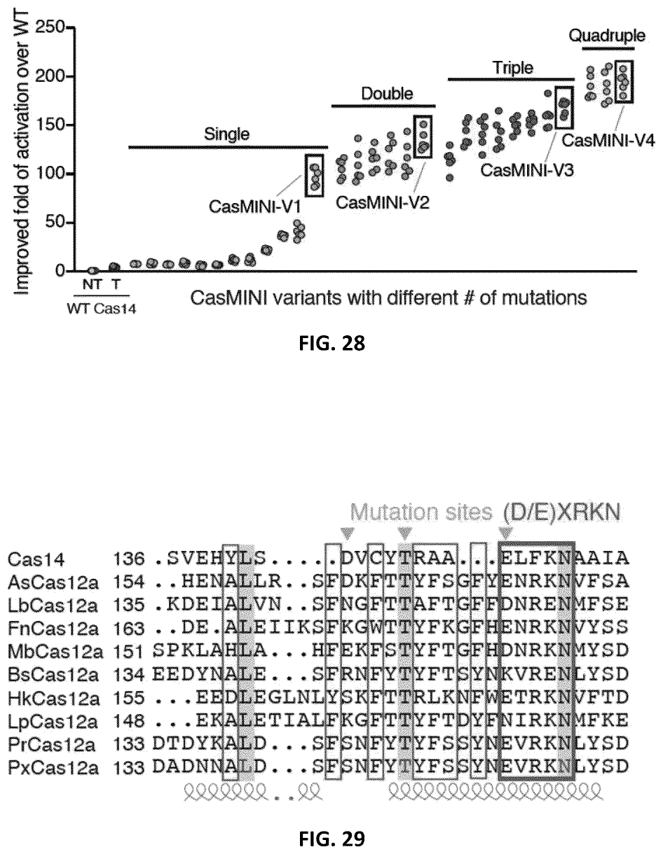

The iterative screening altogether yielded a gradually improved Cas14 variant library (FIG. 28). Interestingly, single mutations at D143, T147, and E151 showing improved activity are all at positions close to a (D/E)XRKN (SEQ ID NO: 253) motif that is highly conserved in the Cas12a family (FIG. 29). These results demonstrate that this domain is a likely hotspot for enhancing Cas14-DNA interaction.

### Example 4. CasMINI Activation of Endogenous Genes

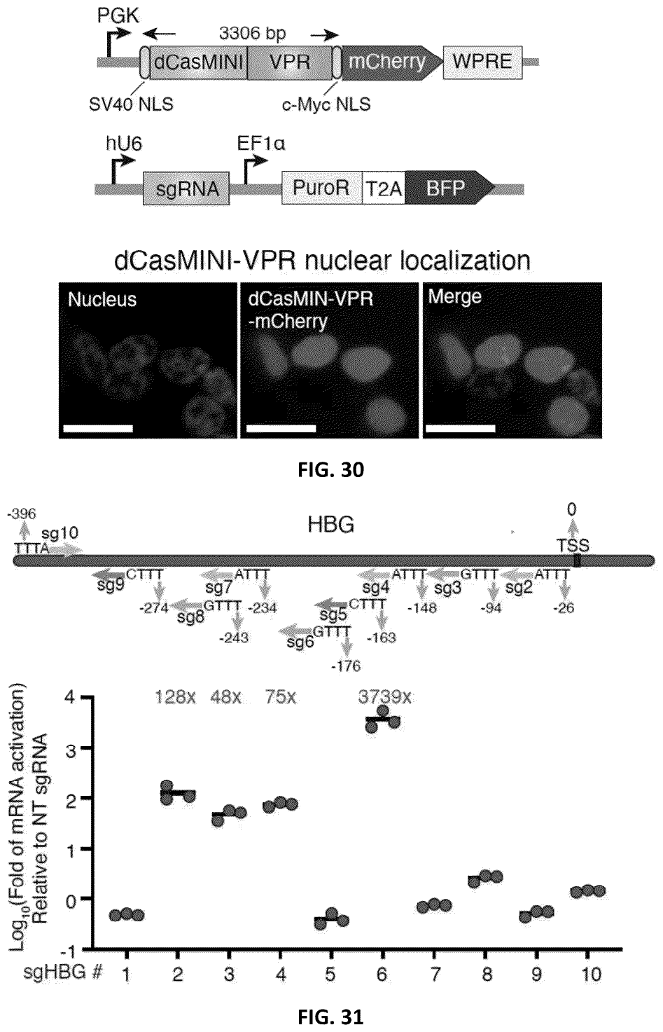

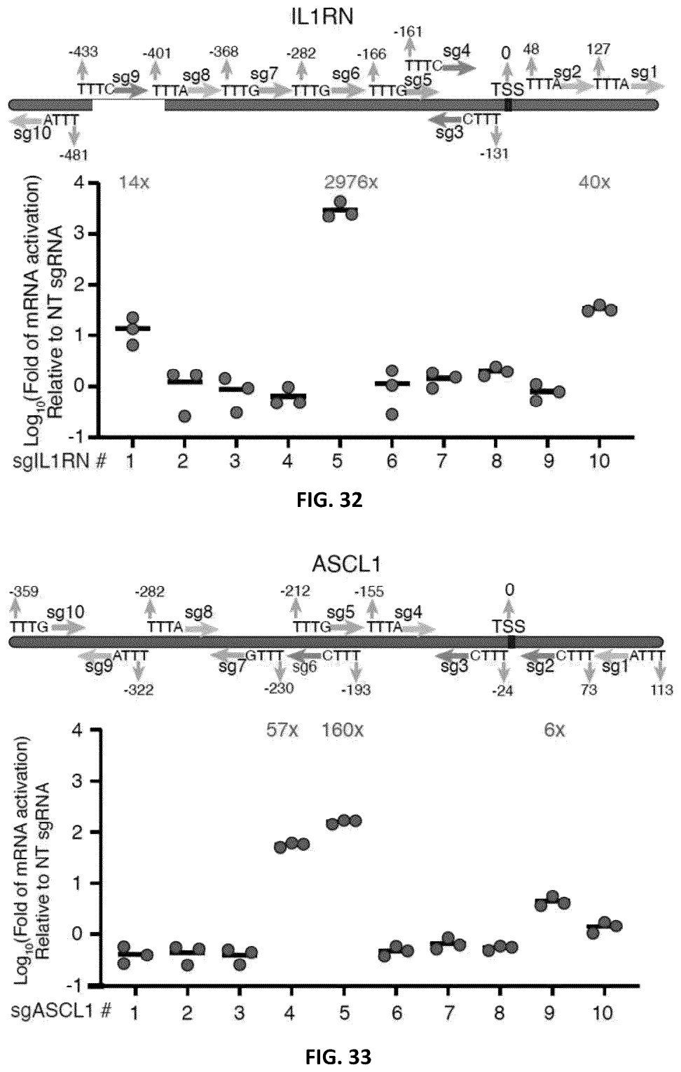

To test whether the engineered protein of Example 3 can activate endogenous genes, dCasMINI was fused to VPR, an N-terminal SV40 NLS, a C-terminal MYC NLS, and mCherry. Nuclear localization was confirmed via confocal microscope fluorescence imaging (FIG. 30). HEK293T cells transduced by dCasMINI-VPR lentivirus was seeded in a 96-well μ-plate (Ibidi, Inc.). Cells were stained with Hoechst 33342 (Thermo Fisher Scientific) to label nucleus at 37° C. for 10 min. Confocal microscopy was performed with a Nikon Spinning Disk Confocal microscope with TIRF. Activation of endogenous genes including HBG, IL1RN, and ASCL1 was tested in the HEK293T cells. For each gene, a panel of 10 sgRNAs was designed, each with different protospacer adjacent motifs (PAMs, TTTA, TTTC, or TTTG) and binding orientation, targeting within 500 bp upstream from the transcriptional start site (TSS) (T. Karvelis et al., Nucleic Acids Res. 48, (2020): 5016-23) (FIGS. 31-33). Oligos for targeting spacers were annealed and ligated into BsmBI digested backbone vectors.

Testing across these sgRNAs showed that gene activation was highly dependent on the sgRNA targeting site. For all three genes, approximately 20-40% of tested sgRNAs showed significant activation, with the best sgRNA activating the target gene by hundreds to thousands of fold improvement of activation. TTTG and TTTA PAMs worked best while TTTC PAM failed to show activation. The results therefore confirmed that TTTR PAM enabled highly efficient gene activation.

### Example 5. Comparison of CasMINI and Cas14

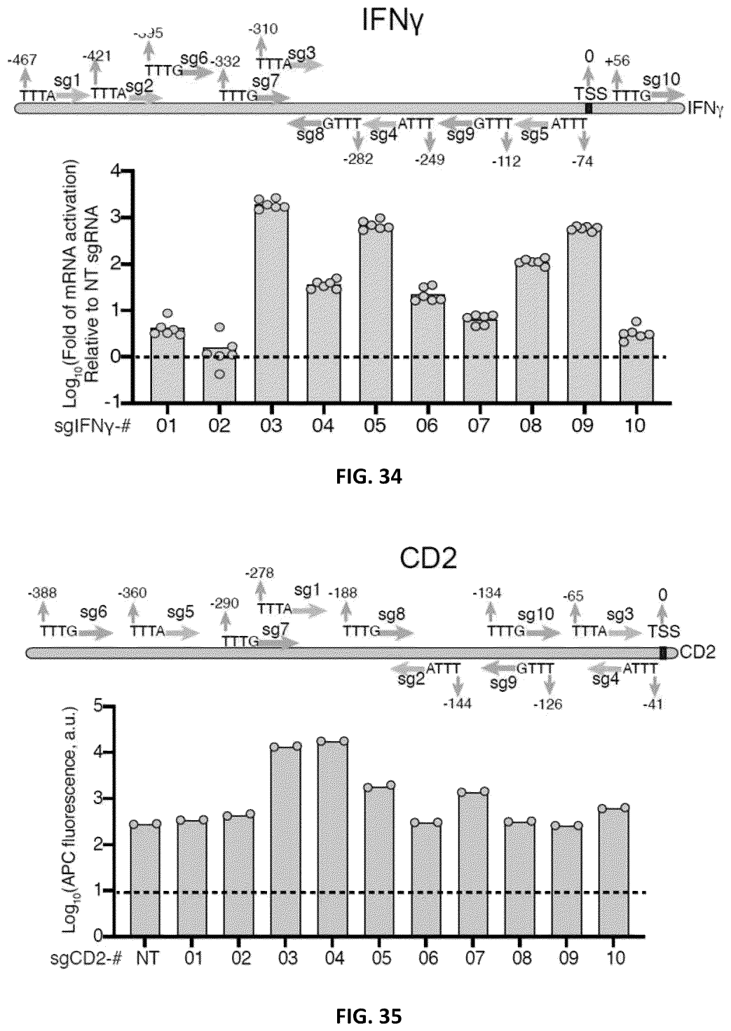

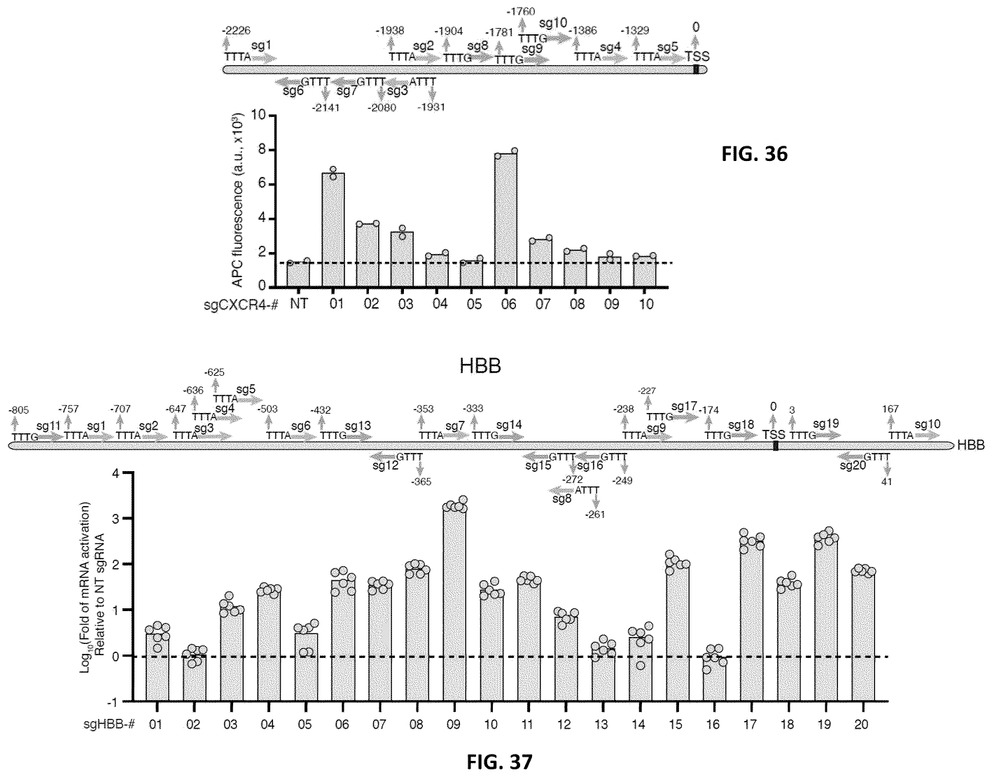

The performance of CasMINI relative to that of wild-type Cas14 for gene activation of a panel of endogenous genes, including IFNγ, HBB, CD2, and CXCR4, was next compared. For IFNγ, CD2, and CXCR4, 10 sgRNAs were designed, and for HBB 20 sgRNAs were designed (FIGS. 34-37). To test activation of the endogenous genes, 800 ng of dCas plasmids and 500 ng sgRNA or crRNA plasmids were transfected to HEK293T cells in 24-well plates. The transfected cells were analyzed 3 days post-transfection for endogenous gene activation. Using the TTTR PAM, it was observed that a large portion of sgRNAs activated the target genes efficiently. The fold improvement of activation on these genes was higher if the genes were silenced (IFNγ, CD2, HBB), which was consistent with what has been reported for dCas9-mediated activation (S. Konermann et al., Nature 517, (2015): 583-88).

Activation of IFNγ and HBB was observed using qPCR. The transfected cells as described above were harvested using Accutase (STEMCELL), and total RNA was extracted using RNeasy Plus Mini Kit (Qiagen). cDNA was prepared using iScript cDNA Synthesis kit (Bio-Rad) and stored at −80° C. qPCR reactions were prepared in 384-well plates with iTaq Universal SYBR Green Supermix (Bio-Rad) and run on a CFX384 Touch Real-Time PCR thermocycler (BioRad). All primers were purchased from IDT. Any Cq values over 35 were considered to be 35, as there were fluctuations for transcripts with weak expression level. Samples transfected with non-targeting sgRNA or crRNA plasmids were used as negative controls. The relative expression fold improvements were analyzed using the ΔΔCq method. The levels of activation fold improvement over negative controls were normalized to the expression of GAPDH.

Activation of IFNγ was also observed using ELISA. Supernatants from transfected cell cultures were harvest 3 days post-transfection, and stored at −80° C. The secreted protein was quantified using the ELISA MAX Deluxe kits for human IFNγ on a Synergy H1 plate reader (BioTek). Absorbance at 450 nm and 570 nm was measured and protein concentrations were determined by the standard curve fitted to a power law.

Activation of CD2 and CXCR4 was observed using immunostaining followed by flow cytometry. Cells were dissociated using Accutase (STEMCELL) and stained in 5% FBS in PBS at 4° C. for 30 min. Antibodies and relevant isotypes of CD2 and CXCR4 were purchase from BioLegend (#309224, #306510, #400122, #400220).

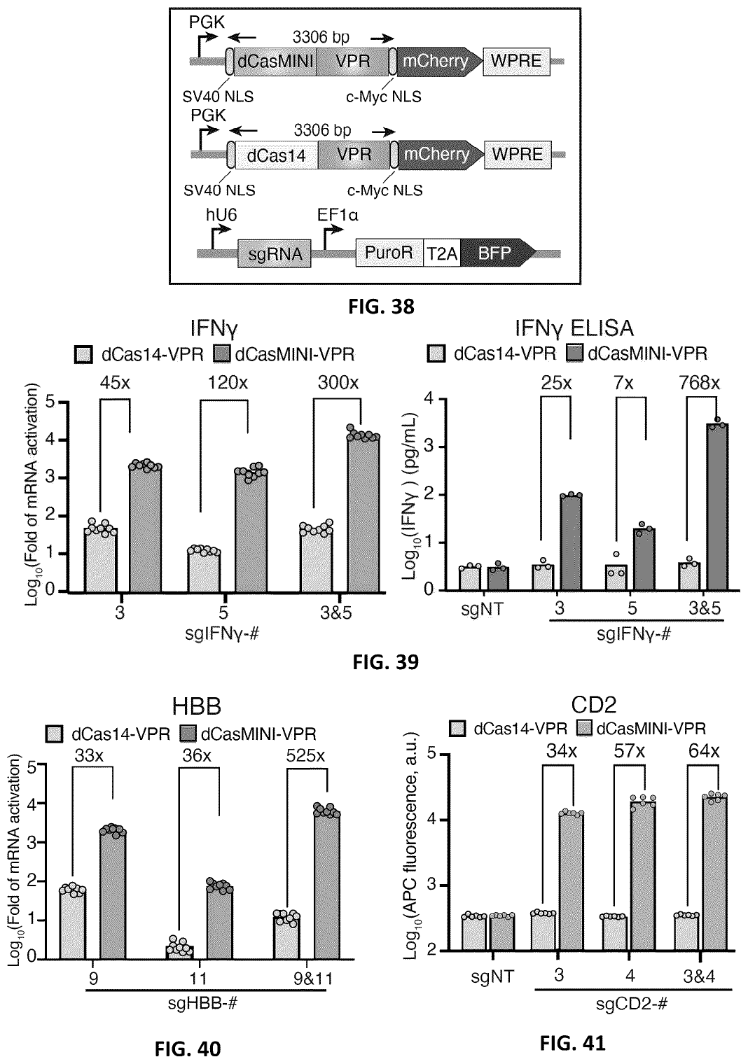

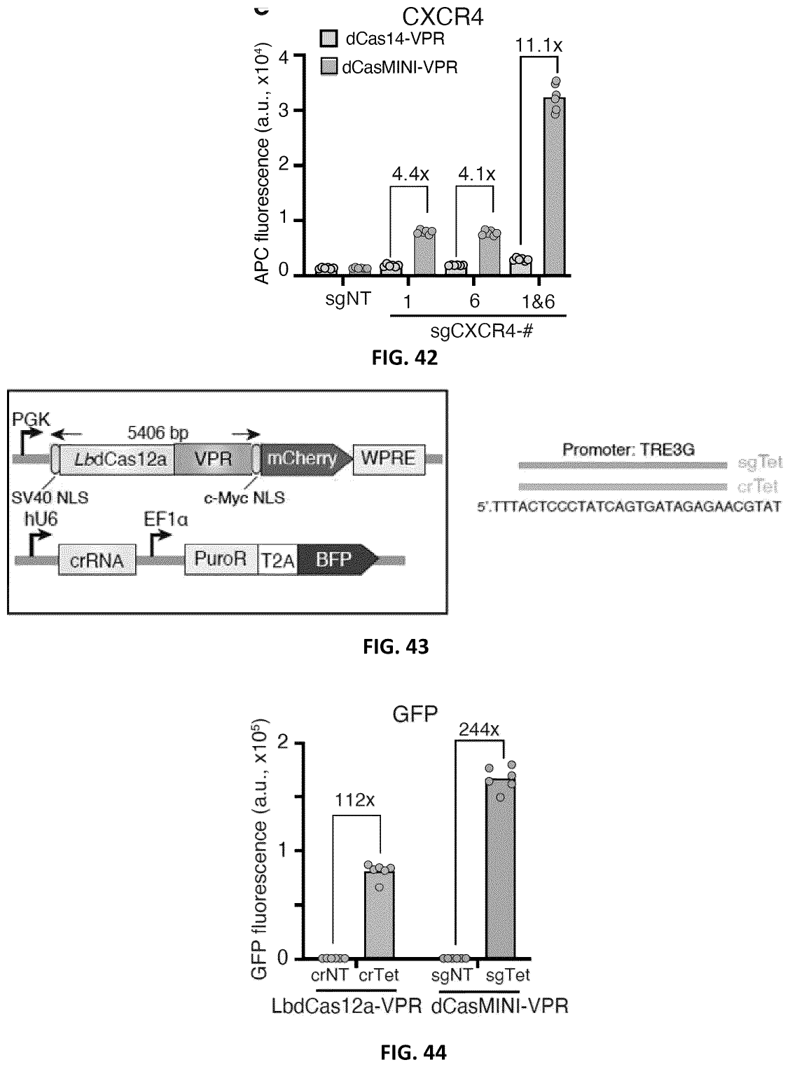

The top sgRNAs were selected for each gene and used to compare dCasMINI-VPR and dCas14-VPR side-by-side (FIG. 38). For all sgRNAs tested, consistent and greatly enhanced activation were observed for each gene. For example, comparing dCasMINI-VPR to dCas14-VPR 45- or 120-fold improvement was observed for IFNγ activation using two different sgRNAs measured by quantitative PCR (qPCR), and 25-fold or 7-fold improvement measured by enzyme-linked immunosorbent assay (ELISA) (FIG. 39). When co-delivering both sgRNAs, even better activation was observed, with improvements that were measured at 300-fold improvement by qPCR and 768-fold improvement by ELISA. Similar improvement was observed for HBB, CD2, and CXCR4 measured by qPCR or flow cytometry: dCasMINI-VPR showed up to 525-fold improvement of HBB activation, 64-fold improvement of CD2 activation, and 11-fold improvement of CXCR4 activation (FIGS. 40-42). The relative lower activation on CXCR4 was likely due to the already high expression level of CXCR4 in HEK293T cells.

### Example 6. Comparison of CasMINI and LbCas12

Further experiments tested whether CasMINI performed equally well to LbCas12a. The Cas12a system is a large Cas effector (1228 amino acids), which is more than twice the size of CasMINI (FIG. 43). The Cas12a effector was chosen as a comparator because it shares a similar PAM (TTTV) as dCasMINI (TTTR), making it fair to compare the performance of two systems side-by-side using the sgRNAs targeting the same genomic site. Four genes—GFP, IFNγ, HBB, and CXCR4—were chosen, and Cas12a crRNAs were designed for binding to the same target sites as those of the best performing sgRNA of CasMINI. Nuclease-deactivated LbdCas12a and its crRNA backbone were amplified as described previously (H. R. Kempton, L. E. Goudy, K. S. Love, & L. S. Qi, Mol. Cell 78, (2020): 184-91).

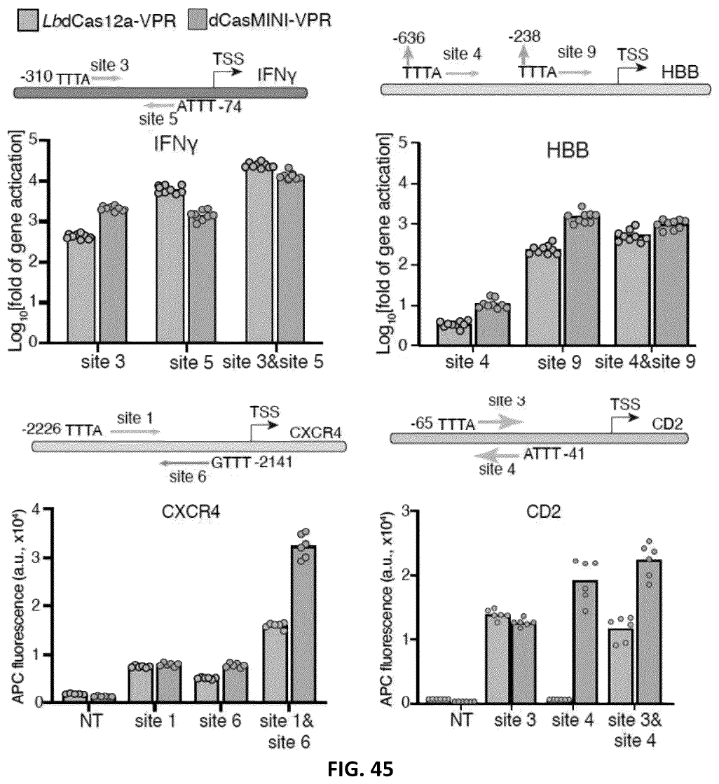

Results indicated that dCasMINI-VPR outperformed dCas12a-VPR for GFP activation by 2-fold (244-fold vs. 112-fold of activation, FIG. 44). Further, for most endogenous genes dCasMINI-VPR was observed to outperformed dCas12a-VPR, suggesting that the CasMINI system is comparable or better than Cas12a for gene activation (FIG. 45). Recent structural studies suggested that Cas14 forms a dimer when binding to the target DNA (S. N. Takeda et al., Mol. Cell 81, (2021): 558-70). It is theorized that for each target site a dimer of dCasMINI-VPR binds to the target site and enhances activation. These findings therefore demonstrate that CasMINI is a small effector comparable to or more effective than the Cas12a system (Y. E. Tak et al., Nat. Methods 14, (2017): 1163-66).

### Example 7. Evaluation of CasMINI Specificity in the Mammalian Genome

To test whether CasMINI is specific in the mammalian genome context, whole-transcriptome RNA sequencing (RNA-seq) in HEK293T cells. The TRE3G-GFP HEK293T reporter cell line was transfected with the dCas and relevant sgRNA or crRNA plasmids and purified based on the expression of fluorescence proteins (mCherry and BFP) 2 days post-transfection. Total RNA was isolated using RNeasy Plus Mini Kit (Qiagen). RNA sequencing library preparation and next-generation sequencing were conducted by Novogene Corporation (Chula Vista, CA). The libraries were sequenced on a NovoSeq6000 platform. Paired-end 150-bp reads were acquired and aligned to the hg38 genome with added GFP using STAR. Transcript abundances were estimated using STAR and htseq using the quantmode option. The counts were imported with tximport, and then normalized and statistically compared using DESeq2. hg38 annotations were downloaded from Gencode (J. Harrow et al., Genome Res. 22, (2012): 1760-74). Custom R Scripts were used to perform further tpm normalization and quality control. Downstream plots used the pheatmap, ggplot2, and tidyverse packages including a custom modified EnhancedVolcano function. The variation of LbdCas12a-VPR versus dCasMINI-VPR systems were represented in violin plots by considering the distribution of standard deviations for gene expression across the four replicates (two targeting and two non-targeting replicates). Linear models and correlation coefficients were obtained using QR decomposition and regression

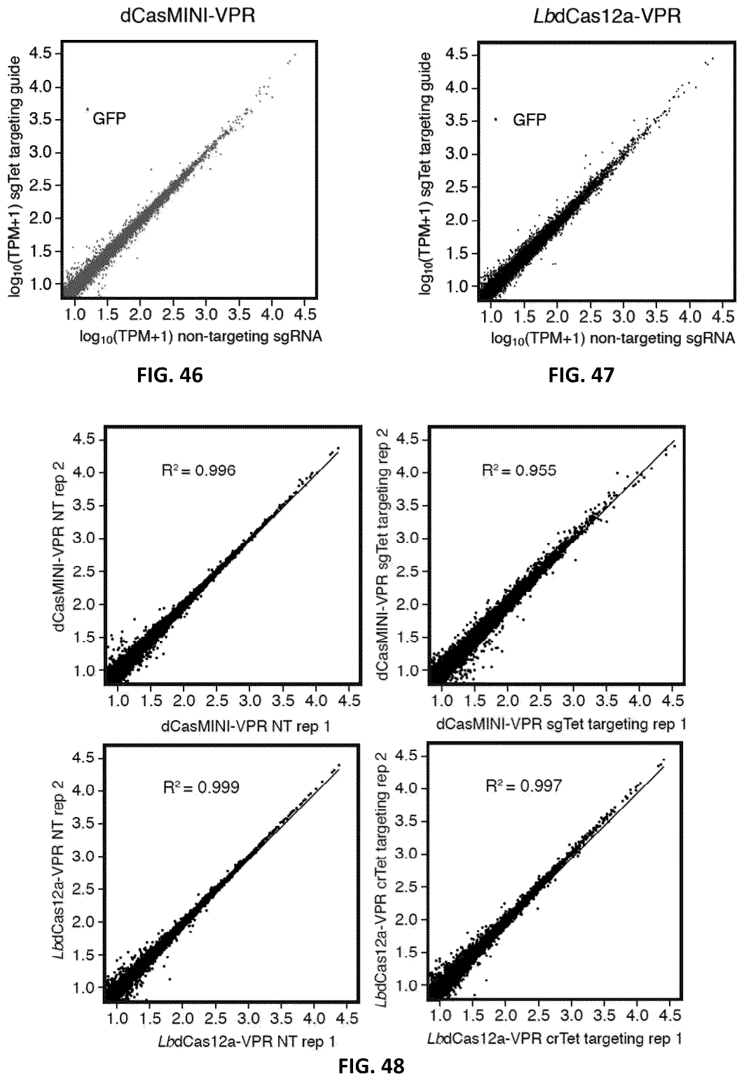

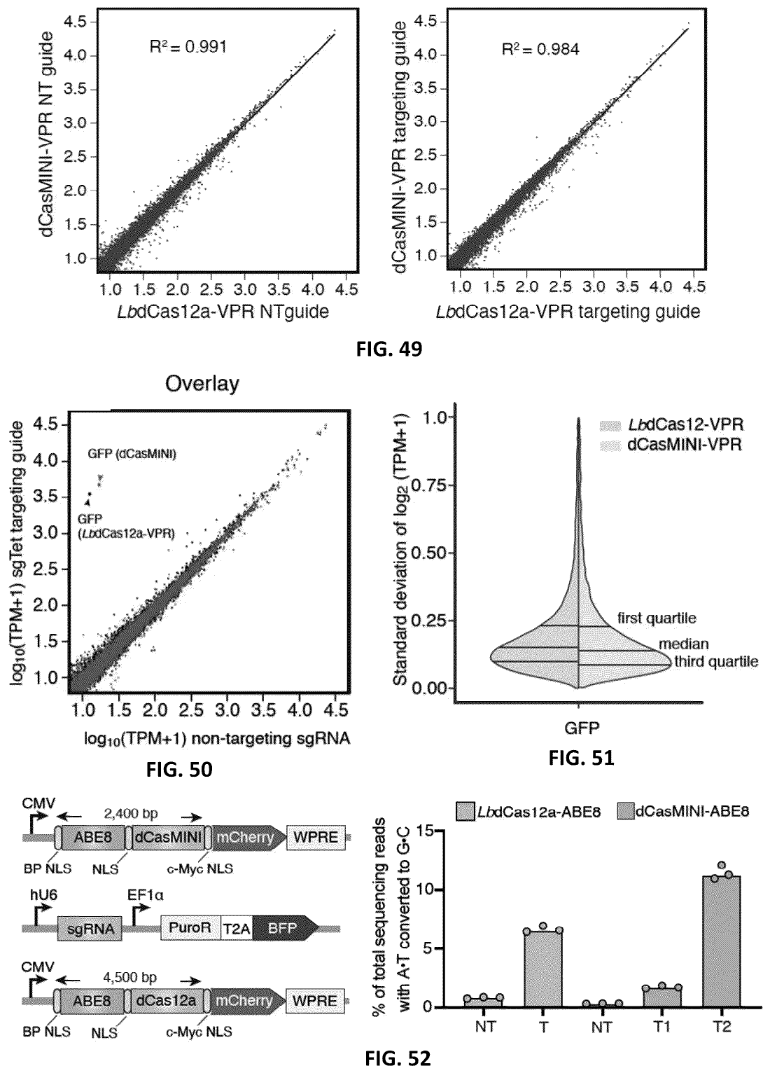

In these experiments, prepared HEK293T cells were transfected with dCasMINI-VPR with a targeting or non-targeting sgRNA, and dCas12a-VPR was used with a targeting and non-targeting sgRNA for comparison (FIGS. 46 and 47). The two biological replicates showed consistent RNA-seq profiling (FIGS. 48 and 49). For both dCasMINI-VPR and dCas12a-VPR, no significant off-target effects comparing the targeting sgRNA to the non-targeting sgRNA were detected. Overlaying the RNA-seq data of dCas12a and dCasMINI (two duplicates shown) demonstrated dCasMINI activated GFP with a better efficiency (FIG. 50). The violin plot of the both datasets also confirmed the two Cas effectors had similar off-target profiles for gene activation (D. Kim et al., Nat. Biotechnol. 34, (2016): 863-68) (FIG. 51). These data together demonstrate the high specificity of using CasMINI in mammalian cells.

### Example 8. Application of CasMINI to Base Editing in Mammalian Cells

Previously developed base editing systems derived by fusing Cas9 or Cas12 with base editors are too large to fit into the packaging capacity of AAV (˜4.5 kb) (M. F. Richter et al., Nat. Biotechnol. 38, (2020): 883-91; X. Li et al., Nat. Biotechnol. 36, (2018): 324-27). In contrast, the greatly reduced size of CasMINI, permits it to reasonably fit it into this size limit. A fusion of the dCasMINI and an adenine base editor (ABE8e) was constructed, resulting in a compact fusion protein (2.4 kb compared to 4.5 kb using dCas12a). Selected genomic sites for converting A⋅T to G⋅C were tested using both base editors. Deep sequencing of edited cells was used to measure the frequency of A⋅T to G⋅C conversion.

For base editing assays, cells were plated at 40,000 cells per well in 48-well plates and transfected using 750 ng of dCas plasmids and 250 ng of sgRNA or crRNA plasmids. The transfected cells were analyzed 3 days post-transfection for endogenous gene activation. The genomic DNA lysate was prepared as described previously (M. F. Richter et al., Nat. Biotechnol. 38, (2020): 883-91) and used as templates for high-throughput sequencing (HTS). Targeted genomic regions of interest were amplified with Q5 Hot Start High-Fidelity Mastermix, 2× (NEB, #M0494S) using two-round PCRs to add Illumina adaptors and unique barcodes for each sample. Libraries were sequenced with on an Illumina Mi-Seq as previously described (M. F. Richter et al., Nat. Biotechnol. 38, (2020): 883-91). CRISPResso2 was used to process fastq.gz files obtained from the Illumina sequencing run. The “min_average_read_quality” flag was set to 30 to filter out reads with average phred33 quality scores less than 30. For each sample, the Alleles_frequency_table.txt was used to quantify the substitution percentage using the following procedure. For each read:amplicon alignment, all columns with an insertion or deletion were removed, and the columns corresponding to the 5 base pairs at the 5′ and 3′ ends of the amplicon were also removed. The quantification region was defined as the columns in the alignment corresponding to the sgRNA as well as 10 columns extending from both ends of the guide. If the sgRNA was reverse complemented with respect to the amplicon, a true mutation is defined as a T-to-C mutation; else, a true mutation is defined as an A-to-G mutation. All other substitutions were defined as random mutations. The substitution percentage was defined as the percentage of remaining reads containing at least one true mutation inside the quantification region.

Results indicated that dCasMINI-ABE showed an editing efficiency comparable to that of dCas12a-ABE (FIG. 52). These findings confirmed that CasMINI can be used in broad genome engineering applications.

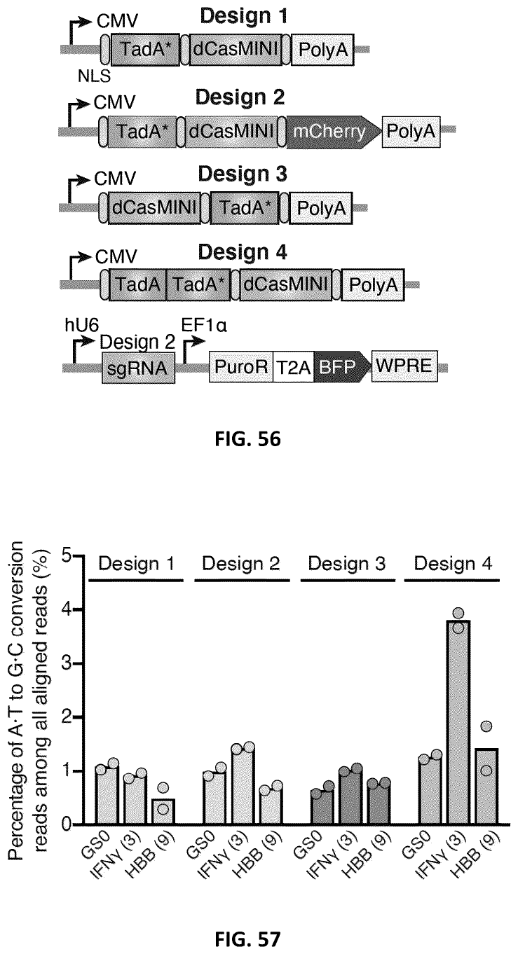

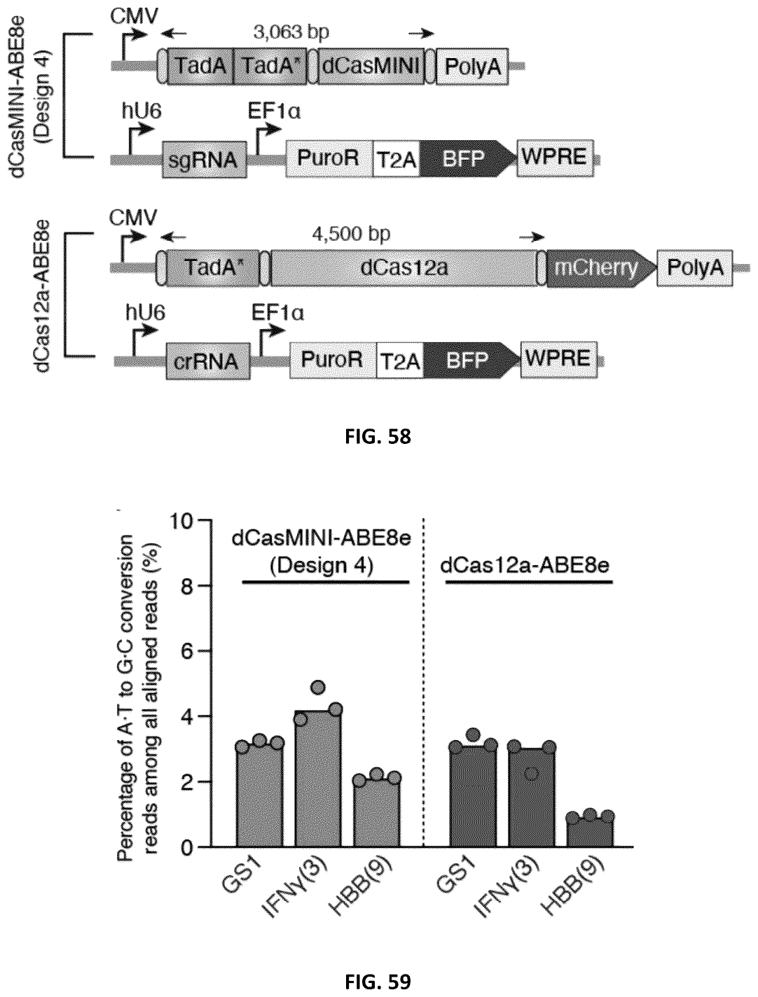

Next, different designs were generated by fusing dCasMINI to the deoxyadenosine deaminase TadA-8e (TadA*) domain or to a heterodimer TadA-TadA* (Design 1-4 in FIG. 56) (T. P. Huang, G. A. Newby, & D. R. Liu, Nat. Protoc. 16, (2021): 1089; M. F. Richter et al., Nat Biotechnol. 38, (2020): 883). The conversion efficiency of A⋅T to G⋅C was measured via high-throughput sequencing (HTS) using these designs at three genomic sites (FIG. 57). Among these protein designs, Design 4 with the TadA-TadA* fusion outperformed others. The frequency of A⋅T to G⋅C conversion using dCasMINI-ABE Design 4 (˜3.0 kb) and dCas12a-ABE (˜4.5 kb) was next compared side-by-side at the same genomic sites and the two systems were found to exhibit similar editing efficiency across these sites (FIGS. 58 and 59).

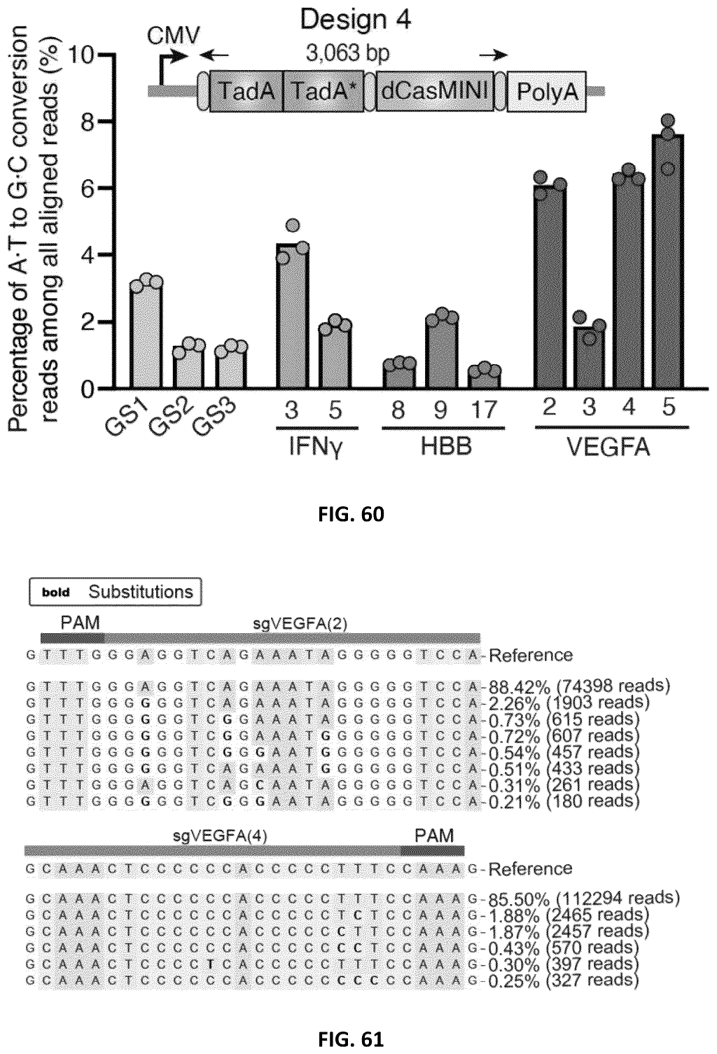

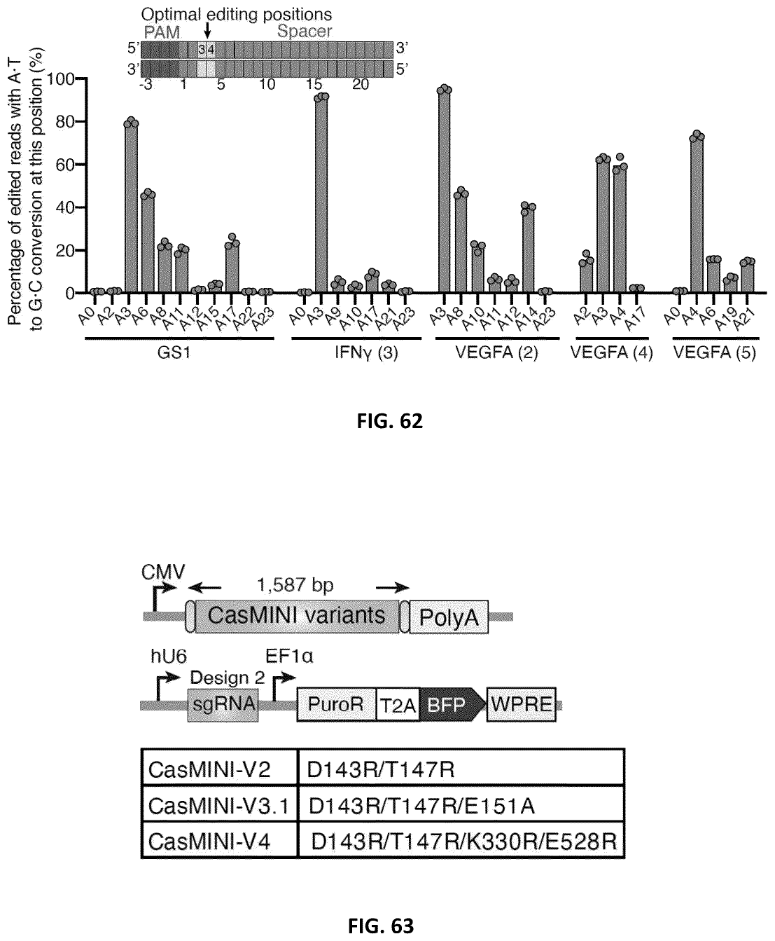

The performance of this fusion for A⋅T to G⋅C base editing was next characterized at a total of 12 genomic sites, including multiples sites in vicinity regions of IFNγ, HBB, and VEGFA loci. For many genomic sites, detectable A⋅T to G⋅C base conversion was observed (FIGS. 60 and 61). The base editing efficiency was dependent on the target site, and the pattern for A⋅T to G⋅C conversion was further analyzed. Interestingly, it was observed that most efficient A⋅T to G⋅C editing occurred in a narrow window A3-A4 (3-4 bp downstream of the PAM, the ‘R’ in the TTTR PAM is position ‘0’) (FIG. 62), suggesting that careful sgRNA target design is needed for efficient base editing.

### Example 9. Application of CasMINI to Transcriptional Repression in Mammalian Cells

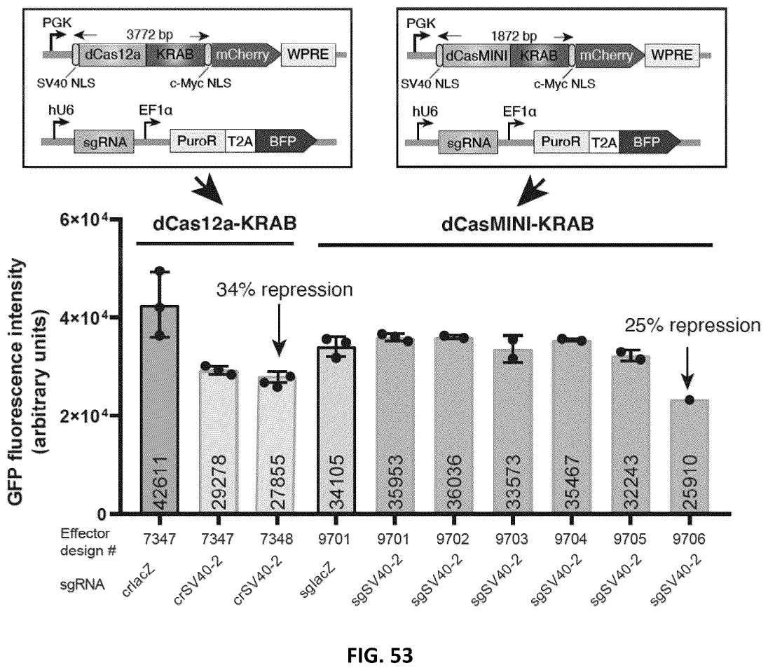

To evaluate the functionality of the provided CasMINI systems as transcriptional repressors, dCas12a and dCasMINI (having 4 mutations) were each independently fused to the transcriptional repressor domain KRAB. The resulting fusions were then tested using a single sgRNA targeting the same site on a synthetic reporter system (SV40 promoter driving EGFP, stably inserted into the genome) in HEK293T cells. Observation of the cells transfected with both dCas12a-KRAB+crRNA or dCasMINI-KRAB+sgRNA, resulted in detection of moderate but statistically significant repression of the target GFP gene. As shown in FIG. 53, dCas12a (yellow bars) exhibited up to 34% repression compared to a non-targeting crRNA (grey bar). dCasMINI (blue bars) exhibited up to 25% repression of the target GFP compared to its control group with the non-targeting sgRNA (light grey). These results demonstrate that the provided systems can be used for transcriptional or epigenetic repression of endogenous genes in the genome.

### Example 10. Application of CasMINI to Gene Editing in Mammalian Cells

To further evaluate the ability of the provided CasMINI systems to edit genes, three constructs were created. Two of these, CasMINI-V2_E2 and CasMINI-V2_E3, included a Cas protein variant having two substitutions in the Cas14 amino acid sequence, and one, CasMINI-V4_E4, included a Cas protein variant having four substitutions. The E2 construct included a 3xFLAG® tag, while the E3 and E4 constructs included a human influenza hemagglutinin (HA) tag. Additionally, 11 different sgRNAs were constructed as shown in Table 1 below. Each guide RNA targeted either TTTG or TTTA PAMs of template or non-template DNA strands, and the genes DNMT1 or VEGFA of the human genome. For each locus, a PCR primer was designed for performing deep sequencing to quantify the percentage of variants for each mutation, e.g., indels, including insertions and deletions.

TABLE 1
sgRNA constructs.

| | Relevant | | | |
|---|---|---|---|---|
| Number | gene | Guide name | Guide sequence | PAM |
| 1 | Human | sgDNMT1- | gctcagcaggcacctgcctcagc | TTTG |
| | DNMT1 | 02 | (SEQ ID NO: 162) | |
| 2 | Human | sgDNMT1- | tttcccttcagCTAAAATAAAGG | TTTA |
| | DNMT1 | 04 | (SEQ ID NO: 163) | |
| 3 | Human | sgDNMT1- | Gctgaagggaaataaaaggaaaa | TTTA* |
| | DNMT1 | 05 | (SEQ ID NO: 164) | |
| 4 | Human | sgDNMT1- | ACATGTCAATCTGTCCGTTCACA | TTTA |
| | DNMT1 | 07 | (SEQ ID NO: 165) | |
| 5 | Human | sgVEGFA- | ggaagtgtccagggatgcttccc | TTTG* |
| | VEGFA | 01 | (SEQ ID NO: 166) | |
| 6 | Human | sgVEGFA- | atgtctgcaggccagatgagggc | TTTG |
| | VEGFA | 02 | (SEQ ID NO: 167) | |
| 7 | Human | sgVEGFA- | ggactggagttgcttcatgtaca | TTTG* |
| | VEGFA | 03 | (SEQ ID NO: 168) | |
| 8 | Human | sgVEGFA- | ggaggtcagaaatagggggtcca | TTTG |
| | VEGFA | 04 | (SEQ ID NO: 169) | |
| 9 | Human | sgVEGFA- | ctcctggaccccctatttctgac | TTTG* |
| | VEGFA | 05 | (SEQ ID NO: 170) | |
| 10 | Human | sgVEGFA- | gaaagggggtggggggagtttgc | TTTG* |
| | VEGFA | 06 | (SEQ ID NO: 171) | |
| 11 | Human | sgVEGFA- | gccagagccggggtgtgcagacg | TTTA |
| | VEGFA | 08 | (SEQ ID NO: 172) | |

*non-template strand

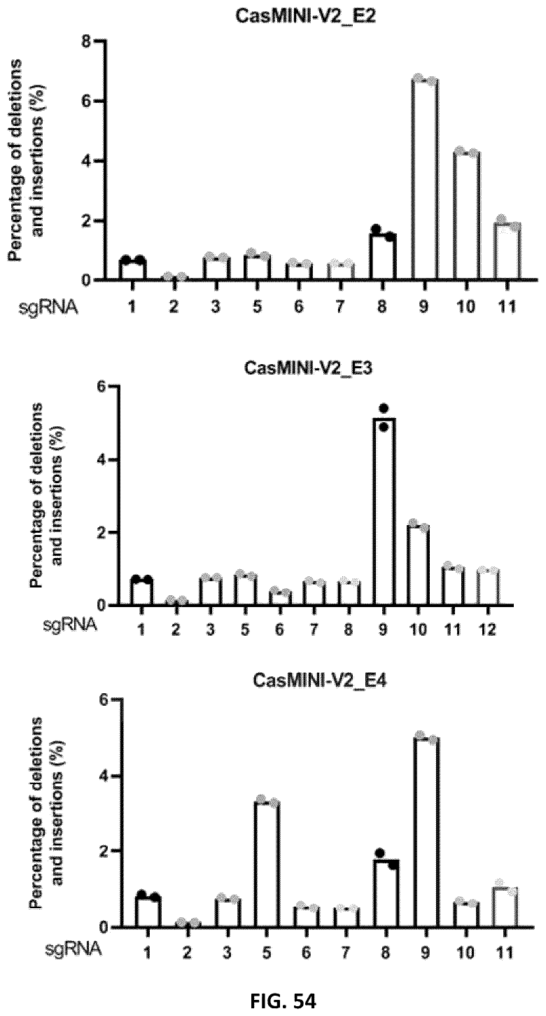

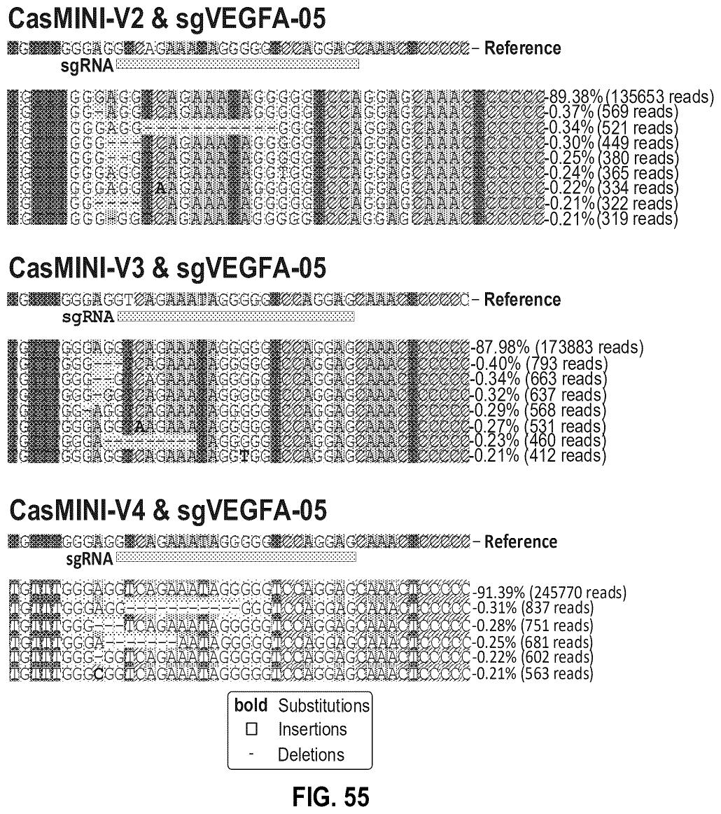

Gene editing results, quantified as the percentage of indels, including deletion, insertion, and substitution allelic mutations in the region within 50 base pairs of the sgRNA, are presented in FIG. 54. The data show that all tested CasMINI systems can induce indels, and thus, gene editing. Among the 11 tested sgRNAs, 4 could induce measurable indels above the background. Among the tested CasMINI constructs, E2 exhibited slightly better gene editing, with 7% of editing efficiency. These results suggests that the performance of gene editing can be dependent on the sgRNA targeting location. Furthermore, the representative sequences presented in FIG. 54 show that the modifications are biased towards the distal region of the target DNA PAM (protospacer adjacent motif, TTTR) by the sgRNA.

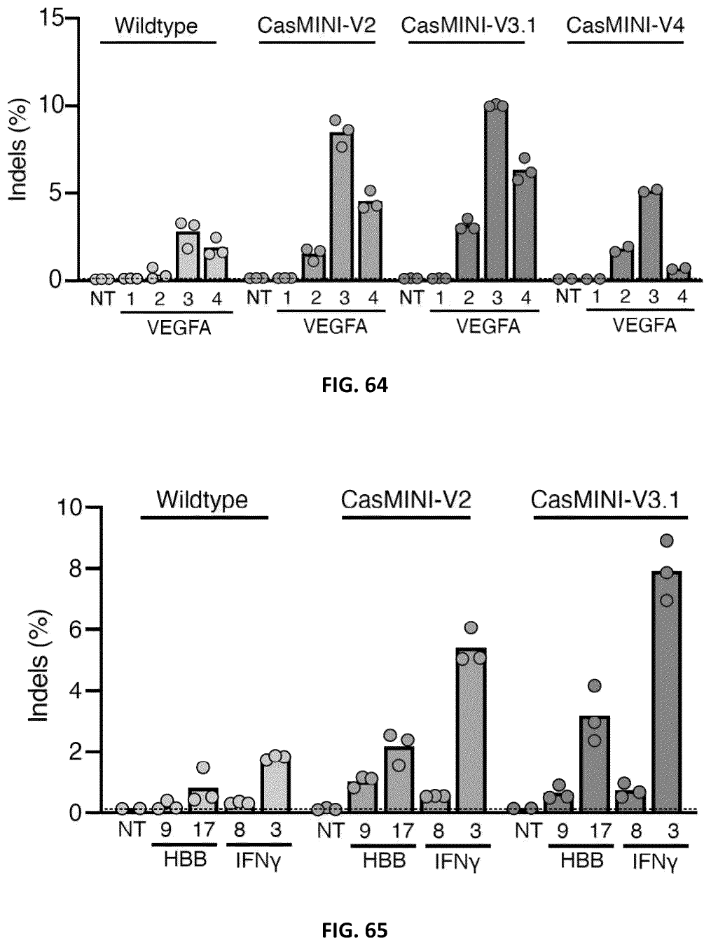

To further investigate whether nuclease-active versions of dCasMINI variants (CasMINI) could cut and edit genomic DNA in human cells, CasMINI-V2 (D143R/T147R), V3.1 (D143R/T147R/E151A), and V4 (D143R/T147R/K330R/E528R) were compared side-by-side with the wild type Cas12f (FIG. 63). The V2 and V3.1 variants were included due to suspicion that the proximity of the K330R and E528R mutations to the catalytic sites in the RuvC domains might negatively impact the DNA cleavage ability of CasMINI-V4. Using the sgRNA Design 2, all variants were tested by targeting four selected sites in the VEGFA genomic locus and measured indel (insertion/deletion) formation efficiency via deep sequencing. Interestingly, it was observed that CasMINI-V3.1 outperformed V2 and V4, which showed consistently higher indel formation across all tested sites (FIG. 64). The use of Design 2 sgRNA also enabled modest indel formation with the wild type Cas12f in mammalian cells, which has not been observed before.

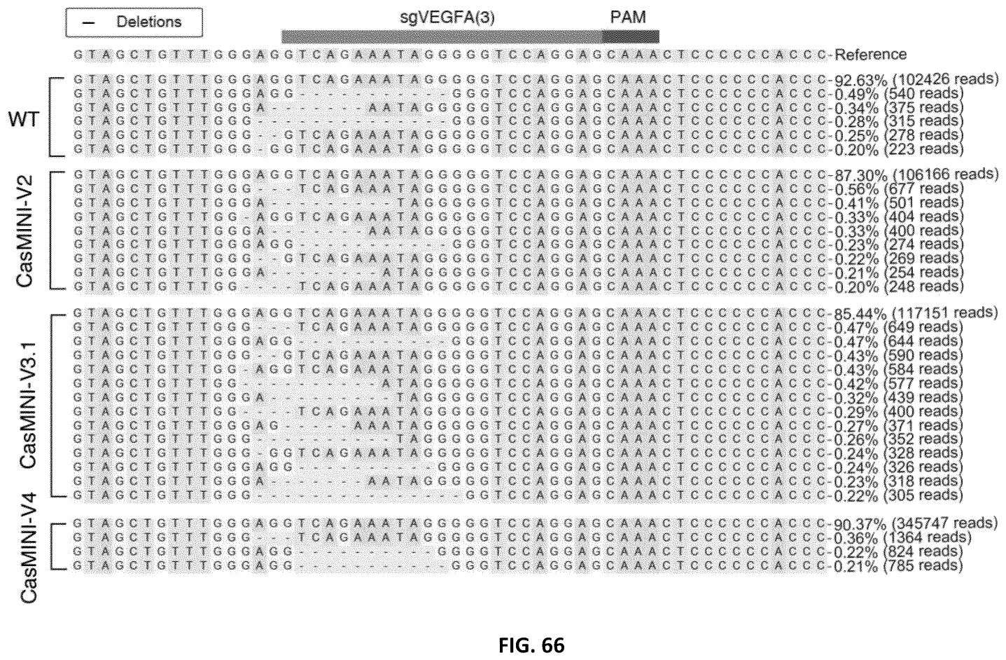

To further characterize gene editing using CasMINI-V3.1, the indel formation efficiency was quantified at four additional genomic sites in HBB or IFNγ (FIGS. 34 and 39). Robust gene editing was observed using CasMINI-V3.1 at these sites, which was more efficient than the wild type Cas12f or CasMINI-V2 (FIGS. 65 and 66). These data suggest that the CasMINI variants enabling optimal gene editing can be different from those used for best gene activation.

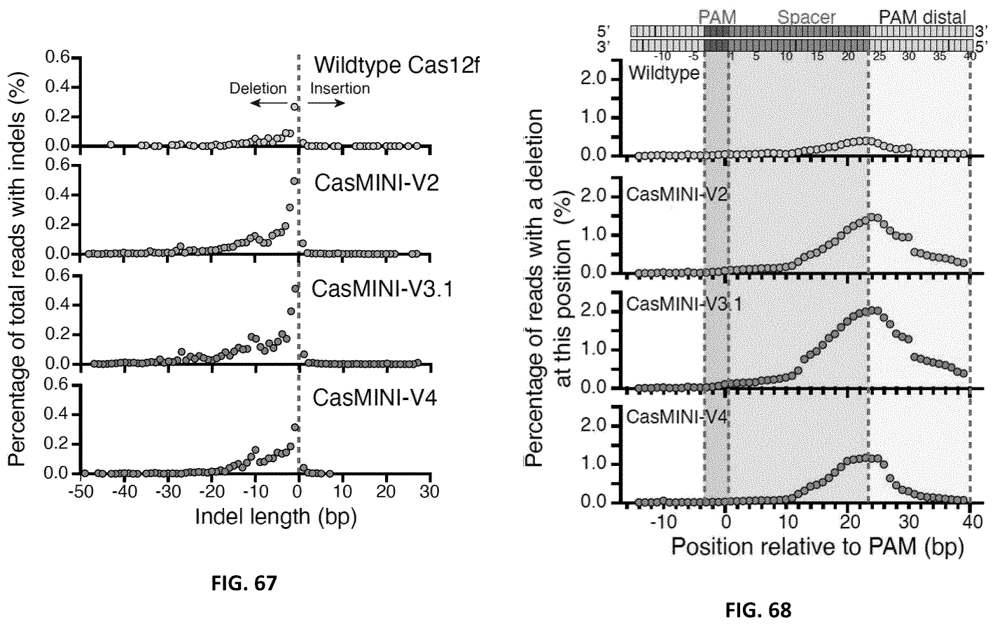

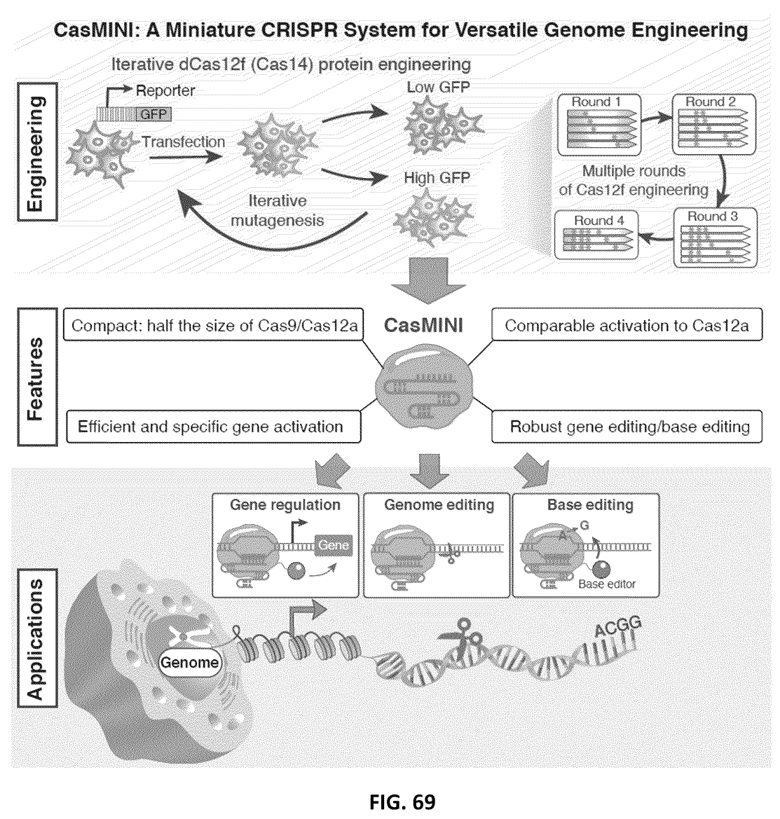

The indel patterns formed by the CasMINI variants were further analyzed by averaging the indel length at top genomic sites. Compared to the wild type Cas12f, CasMINI-V3.1 showed larger deletions (around 20 bp), which was also larger than was reported for Cas9 (FIG. 67) (B. P. Kleinstiver, Nat. Biotechnol. 37, (2019): 276; J. Strecker et al., Nat. Commun. 10, (2019): 212). Indel formation frequency at each nucleotide position was also evaluated. Interestingly, major indel editing was observed at the PAM-distal region spanning outside of the sgRNA-binding sequence (FIG. 68). Previous in vitro assays showed that Cas12f cleavage predominantly centered around positions 20-24 bp relative to the PAM sequence (T. Karvelis et al., Nucleic Acids Res. 48, (2020): 5016). Consistently, our results using CasMINI showed that in vivo gene editing also peaked around positions 20-30 bp relative to PAM (FIG. 68). We thus confirm that CasMINI can be used in broad genome editing applications in addition to gene activation.

### XI. Informal Sequence Listing

| SEQ | | |
|---|---|---|
| ID NO | Sequence | Description |
| 1 | MAKNTITKTLKLRIVRPYNSAEVEKIVADEKNNREKIALEKNKDKVKEACSKHLKVAAYCTT | Cas14 |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 2 | MAKNTITKTLKLRIVRPYNSAEVEKIVADEKNNREKIALEKNKDKVKEACSKHLKVAAYCTT | Cas14/D143R |
| | QVERNACLFCKARKLDDKFYQKLRGQFPDAVFWQEISEIFRQLQKQAAEIYNQSLIELYYEI | (CasMINI-V1) |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 3 | MAKNTITKTLKLRIVRPYNSAEVEKIVADEKNNREKIALEKNKDKVKEACSKHLKVAAYCTT | Cas14/D143R/T147R |
| | QVERNACLFCKARKLDDKFYQKLRGQFPDAVFWQEISEIFRQLQKQAAEIYNQSLIELYYEI | (CasMINI-V2) |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 4 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | K330R (Cas-MINI-V3) |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 5 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | K330R/E528R (Cas- |
| | FIKGKGIANASSVEHYLSRVCYRRAAELFKNAAIASGLRSKIKSNFRLKELKNMKSGLPTTKS | MINI-V4) |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKERP | |
| 6 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | K330R/E151A/E528R |
| | FIKGKGIANASSVEHYLSRVCYRRAAALFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKERP | |
| 7 | 5′GGGCTTCACTGATAAAGTGGAGAACCGCTTCACCAAAAGCTGTCCCTTAGGGGATT | Wild-type sgRNA |
| | AGAACTTGAGTGAAGGTGGGCTGCTTGCATCAGCCTAATGTCGAGAAGTGCTTTCTTC | |
| | GGAAAGTAACCCTCGAAACAAATTCATTTTTCCTCTCCAATTCTGCACAAGAAAGTTGC | |
| | AGAACCCGAATAGACGAATGAAGGAATGCAACNNNNNNNNNNNNNNNNNNNNNN | |
| | N3′ | |
| 8 | 5′GGGCTTCACTGATAAAGTGGAGAACCGCTTCACCAAAAGCTGTCCCTTAGGGGATT | sgRNA design 1 |
| | AGAACTTGAGTGAAGGTGGGCTGCTTGCATCAGCCTAATGTCGAGAAGTGCTTTCTTC | |
| | GGAAAGTAACCCTCGAAACAAATTCATTGTTCCTCTCCAATTCTGCACAAGAAAGTTGC | |
| | AGAACCCGAATAGACGAATGAAGGAATGCAACNNNNNNNNNNNNNNNNNNNNNN | |
| | N3′ | |
| 9 | 5′GGGCTTCACTGATAAAGTGGAGAACCGCTTCACCAAAAGCTGTCCCTTAGGGGATT | sgRNA design |
| | AGAACTTGAGTGAAGGTGGGCTGCTTGCATCAGCCTAATGTCGAGAAGTGCTTTCTTC | 2 |
| | GGAAAGTAACCCTCGAAACAAATTCATTTGAATGAAGGAATGCAACNNNNNNNNNN | |
| | NNNNNNNNNNNN3′ | |
| 10 | 5′GGCTTCACTGATAAAGTGGAGAACCGCTTCACCAAAAGCTGTCCCTTAGGGGATTA | sgRNA design 3 |
| | GAACTTGAGTGAAGGTGGGCTGCTTGCATCAGCCTAATGTCGAGAAGTGCTTTCTTCG | |
| | GAAAGTAACCCTCGAAACAAATTCATTTGAATGAAGGAATGCAACNNNNNNNNNNN | |
| | NNNNNNNNNNN3′ | |
| 11 | MAKNTITKTLRLRIVRPYNSAEVEKIVADEKNNREKIALEKNKDKVKEACSKHLKVAAYCTT | Cas14/K11R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 12 | MAKNTITKTLKLRIVRPYNSAEVEKIVADEKNNREKIALEKNKDKVKEACSKHLKVAAYCTT | Cas14/K73R |
| | QVERNACLFCRARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 13 | MAKNTITKTLKLRIVRPYNSAEVEKIVADEKNNREKIALEKNKDKVKEACSKHLKVAAYCTT | Cas14/Q108R |
| | QVERNACLFCKARKLDDKFYQKLRGQFPDAVFWQEISEIFRQLQKRAAEIYNQSLIELYYEIF | |
| | IKGKGIANASSVEHYLSDVCYTRAAELFKNAAIASGLRSKIKSNFRLKELKNMKSGLPTTKSD | |
| | NFPIPLVKQKGGQYTGFEISNHNSDFIIKIPFGRWQVKKEIDKYRPWEKFDFEQVQKSPKPI | |
| | SLLLSTQRRKRNKGWSKDEGTEAEIKKVMNGDYQTSYIEVKRGSKICEKSAWMLNLSIDVP | |
| | KIDKGVDPSIIGGIDVGVKSPLVCAINNAFSRYSISDNDLFHFNKKMFARRRILLKKNRHKRA | |
| | GHGAKNKLKPITILTEKSERFRKKLIERWACEIADFFIKNKVGTVQMENLESMKRKEDSYFNI | |
| | RLRGFWPYAEMQNKIEFKLKQYGIEIRKVAPNNTSKTCSKCGHLNNYFNFEYRKKNKFPHF | |
| | KCEKCNFKENADYNAALNISNPKLKSTKEEP | |
| 14 | MAKNTITKTLKLRIVRPYNSAEVEKIVADEKNNREKIALEKNKDKVKEACSKHLKVAAYCTT | Cas14/T147R |
| | QVERNACLFCKARKLDDKFYQKLRGQFPDAVFWQEISEIFRQLQKQAAEIYNQSLIELYYEI | |
| | FIKGKGIANASSVEHYLSDVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 15 | MAKNTITKTLKLRIVRPYNSAEVEKIVADEKNNREKIALEKNKDKVKEACSKHLKVAAYCTT | Cas14/E151R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAARLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 16 | MAKNTITKTLKLRIVRPYNSAEVEKIVADEKNNREKIALEKNKDKVKEACSKHLKVAAYCTT | Cas14/K154R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFRNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 17 | MAKNTITKTLKLRIVRPYNSAEVEKIVADEKNNREKIALEKNKDKVKEACSKHLKVAAYCTT | Cas14/N155R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKRAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 18 | MAKNTITKTLKLRIVRPYNSAEVEKIVADEKNNREKIALEKNKDKVKEACSKHLKVAAYCTT | Cas14/K196R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVRQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 19 | MAKNTITKTLKLRIVRPYNSAEVEKIVADEKNNREKIALEKNKDKVKEACSKHLKVAAYCTT | Cas14/E241R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFRQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 20 | MAKNTITKTLKLRIVRPYNSAEVEKIVADEKNNREKIALEKNKDKVKEACSKHLKVAAYCTT | Cas14/E289R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIRVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 21 | MAKNTITKTLKLRIVRPYNSAEVEKIVADEKNNREKIALEKNKDKVKEACSKHLKVAAYCTT | Cas14/D318R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVRPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 22 | MAKNTITKTLKLRIVRPYNSAEVEKIVADEKNNREKIALEKNKDKVKEACSKHLKVAAYCTT | Cas14/V327R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDRGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 23 | MAKNTITKTLKLRIVRPYNSAEVEKIVADEKNNREKIALEKNKDKVKEACSKHLKVAAYCTT | Cas14/K330R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 24 | MAKNTITKTLKLRIVRPYNSAEVEKIVADEKNNREKIALEKNKDKVKEACSKHLKVAAYCTT | Cas14/E425N |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLNSMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 25 | MAKNTITKTLKLRIVRPYNSAEVEKIVADEKNNREKIALEKNKDKVKEACSKHLKVAAYCTT | Cas14/K457R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLRQYGIEIRKVAPNNTSKTCSKCGHLNNYFNFEYRKKNKF | |
| | PHFKCEKCNFKENADYNAALNISNPKLKSTKEEP | |
| 26 | MAKNTITKTLKLRIVRPYNSAEVEKIVADEKNNREKIALEKNKDKVKEACSKHLKVAAYCTT | Cas14/E462R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIRIRKVAPNNTSKTCSKCGHLNNYFNFEYRKKNKF | |
| | PHFKCEKCNFKENADYNAALNISNPKLKSTKEEP | |
| 27 | MAKNTITKTLKLRIVRPYNSAEVEKIVADEKNNREKIALEKNKDKVKEACSKHLKVAAYCTT | Cas14/C500R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKREKCNFKENADYNAALNISNPKLKSTKEEP | |
| 28 | MAKNTITKTLKLRIVRPYNSAEVEKIVADEKNNREKIALEKNKDKVKEACSKHLKVAAYCTT | Cas14/K499R/C500R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFRREKCNFKENADYNAALNISNPKLKSTKEEP | |
| 29 | MAKNTITKTLKLRIVRPYNSAEVEKIVADEKNNREKIALEKNKDKVKEACSKHLKVAAYCTT | Cas14/E501R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCRKCNFKENADYNAALNISNPKLKSTKEEP | |
| 30 | MAKNTITKTLKLRIVRPYNSAEVEKIVADEKNNREKIALEKNKDKVKEACSKHLKVAAYCTT | Cas14/E504R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCRFKENADYNAALNISNPKLKSTKEEP | |
| 31 | MAKNTITKTLKLRIVRPYNSAEVEKIVADEKNNREKIALEKNKDKVKEACSKHLKVAAYCTT | Cas14/E507R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKRNADYNAALNISNPKLKSTKEEP | |
| 32 | MAKNTITKTLKLRIVRPYNSAEVEKIVADEKNNREKIALEKNKDKVKEACSKHLKVAAYCTT | Cas14/N508D |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKEDADYNAALNISNPKLKSTKEEP | |
| 33 | MAKNTITKTLKLRIVRPYNSAEVEKIVADEKNNREKIALEKNKDKVKEACSKHLKVAAYCTT | Cas14/Y511A |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADANAALNISNPKLKSTKEEP | |
| 34 | MAKNTITKTLKLRIVRPYNSAEVEKIVADEKNNREKIALEKNKDKVKEACSKHLKVAAYCTT | Cas14/A514G |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAGLNISNPKLKSTKEEP | |
| 35 | AKNTITKTLKLRIVRPYNSAEVEKIVADEKNNREKIALEKNKDKVKEACSKHLKVAAYCTTQV | Cas14/N516R |
| | ERNACLFCKARKLDDKFYQKLRGQFPDAVFWQEISEIFRQLQKQAAEIYNQSLIELYYEIFIK | |
| | GKGIANASSVEHYLSDVCYTRAAELFKNAAIASGLRSKIKSNFRLKELKNMKSGLPTTKSDNF | |
| | PIPLVKQKGGQYTGFEISNHNSDFIIKIPFGRWQVKKEIDKYRPWEKFDFEQVQKSPKPISLL | |
| | LSTQRRKRNKGWSKDEGTEAEIKKVMNGDYQTSYIEVKRGSKICEKSAWMLNLSIDVPKID | |
| | KGVDPSIIGGIDVGVKSPLVCAINNAFSRYSISDNDLFHFNKKMFARRRILLKKNRHKRAGH | |
| | GAKNKLKPITILTEKSERFRKKLIERWACEIADFFIKNKVGTVQMENLESMKRKEDSYFNIRL | |
| | RGFWPYAEMQNKIEFKLKQYGIEIRKVAPNNTSKTCSKCGHLNNYFNFEYRKKNKFPHFKC | |
| | EKCNFKENADYNAALRISNPKLKSTKEEP | |
| 36 | MAKNTITKTLKLRIVRPYNSAEVEKIVADEKNNREKIALEKNKDKVKEACSKHLKVAAYCTT | Cas14/N519R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISRPKLKSTKEEP | |
| 37 | MAKNTITKTLKLRIVRPYNSAEVEKIVADEKNNREKIALEKNKDKVKEACSKHLKVAAYCTT | Cas14/E527R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKREP | |
| 38 | MAKNTITKTLKLRIVRPYNSAEVEKIVADEKNNREKIALEKNKDKVKEACSKHLKVAAYCTT | Cas14/E528R |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKERP | |
| 39 | MAKNTITKTLKLRIVRPYNSAEVEKIVADEKNNREKIALEKNKDKVKEACSKHLKVAAYCTT | Cas14/D143R/E151R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAARLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 40 | MAKNTITKTLKLRIVRPYNSAEVEKIVADEKNNREKIALEKNKDKVKEACSKHLKVAAYCTT | Cas14/D143R/K154R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFRNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 41 | MAKNTITKTLKLRIVRPYNSAEVEKIVADEKNNREKIALEKNKDKVKEACSKHLKVAAYCTT | Cas14/D143R/E241R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFRQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 42 | MAKNTITKTLKLRIVRPYNSAEVEKIVADEKNNREKIALEKNKDKVKEACSKHLKVAAYCTT | Cas14/D143R/K330R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 43 | MAKNTITKTLKLRIVRPYNSAEVEKIVADEKNNREKIALEKNKDKVKEACSKHLKVAAYCTT | Cas14/D143R/E245N |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLNSMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 44 | MAKNTITKTLKLRIVRPYNSAEVEKIVADEKNNREKIALEKNKDKVKEACSKHLKVAAYCTT | Cas14/D143R/E507R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKRNADYNAALNISNPKLKSTKEEP | |
| 45 | MAKNTITKTLKLRIVRPYNSAEVEKIVADEKNNREKIALEKNKDKVKEACSKHLKVAAYCTT | Cas14/D143R/N516R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALRISNPKLKSTKEEP | |
| 46 | MAKNTITKTLKLRIVRPYNSAEVEKIVADEKNNREKIALEKNKDKVKEACSKHLKVAAYCTT | Cas14/D143R/N519R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISRPKLKSTKEEP | |
| 47 | MAKNTITKTLKLRIVRPYNSAEVEKIVADEKNNREKIALEKNKDKVKEACSKHLKVAAYCTT | Cas14/D143R/E527R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKREP | |
| 48 | MAKNTITKTLKLRIVRPYNSAEVEKIVADEKNNREKIALEKNKDKVKEACSKHLKVAAYCTT | Cas14/D143R/E528R |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKERP | |
| 49 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E151R |
| | FIKGKGIANASSVEHYLSRVCYRRAARLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 50 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | K154R |
| | FIKGKGIANASSVEHYLSRVCYRRAAELFRNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 51 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E241R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFRQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 52 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E425N |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLNSMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 53 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E507R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKRNADYNAALNISNPKLKSTKEEP | |
| 54 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | N516R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALRISNPKLKSTKEEP | |
| 55 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | N519R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISRPKLKSTKEEP | |
| 56 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E527R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKREP | |
| 57 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E528R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKERP | |
| 58 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E151S |
| | FIKGKGIANASSVEHYLSRVCYRRAASLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 59 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E151G |
| | FIKGKGIANASSVEHYLSRVCYRRAAGLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 60 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | E151A |
| | FIKGKGIANASSVEHYLSRVCYRRAAALFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 61 | MAKNTITKTLKLRIVRPYNSAEVEKIVADEKNNREKIALEKNKDKVKEACSKHLKVAAYCTT | Cas14/D143R/E151L |
| | QVERNACLFCKARKLDDKFYQKLRGQFPDAVFWQEISEIFRQLQKQAAEIYNQSLIELYYEI | |
| | FIKGKGIANASSVEHYLSRVCYTRAALLFKNAAIASGLRSKIKSNFRLKELKNMKSGLPTTKSD | |
| | NFPIPLVKQKGGQYTGFEISNHNSDFIIKIPFGRWQVKKEIDKYRPWEKFDFEQVQKSPKPI | |
| | SLLLSTQRRKRNKGWSKDEGTEAEIKKVMNGDYQTSYIEVKRGSKICEKSAWMLNLSIDVP | |
| | KIDKGVDPSIIGGIDVGVKSPLVCAINNAFSRYSISDNDLFHFNKKMFARRRILLKKNRHKRA | |
| | GHGAKNKLKPITILTEKSERFRKKLIERWACEIADFFIKNKVGTVQMENLESMKRKEDSYFNI | |
| | RLRGFWPYAEMQNKIEFKLKQYGIEIRKVAPNNTSKTCSKCGHLNNYFNFEYRKKNKFPHF | |
| | KCEKCNFKENADYNAALNISNPKLKSTKEEP | |
| 62 | MAKNTITKTLKLRIVRPYNSAEVEKIVADEKNNREKIALEKNKDKVKEACSKHLKVAAYCTT | Cas14/D143R/E151S |
| | QVERNACLFCKARKLDDKFYQKLRGQFPDAVFWQEISEIFRQLQKQAAEIYNQSLIELYYEI | |
| | FIKGKGIANASSVEHYLSRVCYTRAASLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 63 | MAKNTITKTLKLRIVRPYNSAEVEKIVADEKNNREKIALEKNKDKVKEACSKHLKVAAYCTT | Cas14/D143R/E151T |
| | QVERNACLFCKARKLDDKFYQKLRGQFPDAVFWQEISEIFRQLQKQAAEIYNQSLIELYYEI | |
| | FIKGKGIANASSVEHYLSRVCYTRAATLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 64 | MAKNTITKTLKLRIVRPYNSAEVEKIVADEKNNREKIALEKNKDKVKEACSKHLKVAAYCTT | Cas14/D143R/E151W |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAWLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 65 | MAKNTITKTLKLRIVRPYNSAEVEKIVADEKNNREKIALEKNKDKVKEACSKHLKVAAYCTT | Cas14/D143R/E151F |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAFLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 66 | MAKNTITKTLKLRIVRPYNSAEVEKIVADEKNNREKIALEKNKDKVKEACSKHLKVAAYCTT | Cas14/D143R/E151C |
| | QVERNACLFCKARKLDDKFYQKLRGQFPDAVFWQEISEIFRQLQKQAAEIYNQSLIELYYEI | |
| | FIKGKGIANASSVEHYLSRVCYTRAACLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 67 | MAKNTITKTLKLRIVRPYNSAEVEKIVADEKNNREKIALEKNKDKVKEACSKHLKVAAYCTT | Cas14/D143R/E151K |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAKLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 68 | MAKNTITKTLKLRIVRPYNSAEVEKIVADEKNNREKIALEKNKDKVKEACSKHLKVAAYCTT | Cas14/D143R/E151G |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAGLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 69 | MAKNTITKTLKLRIVRPYNSAEVEKIVADEKNNREKIALEKNKDKVKEACSKHLKVAAYCTT | Cas14/D143R/E151I |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAILFKNAAIASGLRSKIKSNFRLKELKNMKSGLPTTKSD | |
| | NFPIPLVKQKGGQYTGFEISNHNSDFIIKIPFGRWQVKKEIDKYRPWEKFDFEQVQKSPKPI | |
| | SLLLSTQRRKRNKGWSKDEGTEAEIKKVMNGDYQTSYIEVKRGSKICEKSAWMLNLSIDVP | |
| | KIDKGVDPSIIGGIDVGVKSPLVCAINNAFSRYSISDNDLFHFNKKMFARRRILLKKNRHKRA | |
| | GHGAKNKLKPITILTEKSERFRKKLIERWACEIADFFIKNKVGTVQMENLESMKRKEDSYFNI | |
| | RLRGFWPYAEMQNKIEFKLKQYGIEIRKVAPNNTSKTCSKCGHLNNYFNFEYRKKNKFPHF | |
| | KCEKCNFKENADYNAALNISNPKLKSTKEEP | |
| 70 | MAKNTITKTLKLRIVRPYNSAEVEKIVADEKNNREKIALEKNKDKVKEACSKHLKVAAYCTT | Cas14/D143R/E151P |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAPLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 71 | MAKNTITKTLKLRIVRPYNSAEVEKIVADEKNNREKIALEKNKDKVKEACSKHLKVAAYCTT | Cas14/D143R/E151H |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAHLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 72 | MAKNTITKTLKLRIVRPYNSAEVEKIVADEKNNREKIALEKNKDKVKEACSKHLKVAAYCTT | Cas14/D143R/E151A |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAALFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 73 | MAKNTITKTLKLRIVRPYNSAEVEKIVADEKNNREKIALEKNKDKVKEACSKHLKVAAYCTT | Cas14/D143R/E151V |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAVLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 74 | MAKNTITKTLKLRIVRPYNSAEVEKIVADEKNNREKIALEKNKDKVKEACSKHLKVAAYCTT | Cas14/D143R/E151Q |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAQLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 75 | MAKNTITKTLKLRIVRPYNSAEVEKIVADEKNNREKIALEKNKDKVKEACSKHLKVAAYCTT | Cas14/D143R/E151M |
| | QVERNACLFCKARKLDDKFYQKLRGQFPDAVFWQEISEIFRQLQKQAAEIYNQSLIELYYEI | |
| | FIKGKGIANASSVEHYLSRVCYTRAAMLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 76 | MAKNTITKTLKLRIVRPYNSAEVEKIVADEKNNREKIALEKNKDKVKEACSKHLKVAAYCTT | Cas14/D143R/E151D |
| | QVERNACLFCKARKLDDKFYQKLRGQFPDAVFWQEISEIFRQLQKQAAEIYNQSLIELYYEI | |
| | FIKGKGIANASSVEHYLSRVCYTRAADLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 77 | MAKNTITKTLKLRIVRPYNSAEVEKIVADEKNNREKIALEKNKDKVKEACSKHLKVAAYCTT | Cas14/D143R/E151N |
| | QVERNACLFCKARKLDDKFYQKLRGQFPDAVFWQEISEIFRQLQKQAAEIYNQSLIELYYEI | |
| | FIKGKGIANASSVEHYLSRVCYTRAANLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 78 | MAKNTITKTLKLRIVRPYNSAEVEKIVADEKNNREKIALEKNKDKVKEACSKHLKVAAYCTT | Cas14/D143R/E151Y |
| | QVERNACLFCKARKLDDKFYQKLRGQFPDAVFWQEISEIFRQLQKQAAEIYNQSLIELYYEI | |
| | FIKGKGIANASSVEHYLSRVCYRRAAYLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKEEP | |
| 79 | MAKNTITKTLKLRIVRPYNSAEVEKIVADEKNNREKIALEKNKDKVKEACSKHLKVAAYCTT | Cas14/D143R/T147R/ |
| | QVERNACLFCKARKLDDKFYQKLRGQFPDAVFWQEISEIFRQLQKQAAEIYNQSLIELYYEI | K330R/E151A/E527R |
| | FIKGKGIANASSVEHYLSRVCYRRAAALFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIDVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENADYNAALNISNPKLKSTKREP | |
| 80 | MAKNTITKTLKLRIVRPYNSAEVEKIVADEKNNREKIALEKNKDKVKEACSKHLKVAAYCTT | Cas14/D326A/D510A |
| | QVERNACLFCKARKLDDKFYQKLRGQFPDAVFWQEISEIFRQLQKQAAEIYNQSLIELYYEI | |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 81 | MAKNTITKTLRLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | K11R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 82 | MAKNTITKTLKLRIVRPYNSAEVEKIVADEKNNREKIALEKNKDKVKEACSKHLKVAAYCTT | Cas14/D326A/D510A/ |
| | QVERNACLFCRARKLDDKFYQKLRGQFPDAVFWQEISEIFRQLQKQAAEIYNQSLIELYYEI | K73R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 83 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKRAAEIYNQSLIELYYEIF | Q108R |
| | IKGKGIANASSVEHYLSDVCYTRAAELFKNAAIASGLRSKIKSNFRLKELKNMKSGLPTTKSD | |
| | NFPIPLVKQKGGQYTGFEISNHNSDFIIKIPFGRWQVKKEIDKYRPWEKFDFEQVQKSPKPI | |
| | SLLLSTQRRKRNKGWSKDEGTEAEIKKVMNGDYQTSYIEVKRGSKICEKSAWMLNLSIDVP | |
| | KIDKGVDPSIIGGIAVGVKSPLVCAINNAFSRYSISDNDLFHFNKKMFARRRILLKKNRHKRA | |
| | GHGAKNKLKPITILTEKSERFRKKLIERWACEIADFFIKNKVGTVQMENLESMKRKEDSYENI | |
| | RLRGFWPYAEMQNKIEFKLKQYGIEIRKVAPNNTSKTCSKCGHLNNYFNFEYRKKNKFPHF | |
| | KCEKCNFKENAAYNAALNISNPKLKSTKEEP | |
| 84 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 85 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | T147R |
| | FIKGKGIANASSVEHYLSDVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 86 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E151R |
| | FIKGKGIANASSVEHYLSDVCYTRAARLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 87 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | K154R |
| | FIKGKGIANASSVEHYLSDVCYTRAAELFRNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 88 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | N155R |
| | FIKGKGIANASSVEHYLSDVCYTRAAELFKRAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 89 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | K196R |
| | FIKGKGIANASSVEHYLSDVCYTRAAELFKNAAIASGLRSKIKSNFRLKELKNMKSGLPTTKS | |
| | DNFPIPLVRQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 90 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E241R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFRQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 91 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E289R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIRVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 92 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D318R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVRPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 93 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | V327R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIARGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 94 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | K330R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 95 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E425N |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLNSMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 96 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | K457R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLRQYGIEIRKVAPNNTSKTCSKCGHLNNYFNFEYRKKNKF | |
| | PHFKCEKCNFKENAAYNAALNISNPKLKSTKEEP | |
| 97 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E462R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIRIRKVAPNNTSKTCSKCGHLNNYFNFEYRKKNKF | |
| | PHFKCEKCNFKENAAYNAALNISNPKLKSTKEEP | |
| 98 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | C500R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKREKCNFKENAAYNAALNISNPKLKSTKEEP | |
| 99 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | K499R/C500R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFRREKCNFKENAAYNAALNISNPKLKSTKEEP | |
| 100 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E501R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCRKCNFKENAAYNAALNISNPKLKSTKEEP | |
| 101 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E504R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCRFKENAAYNAALNISNPKLKSTKEEP | |
| 102 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E507R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKRNAAYNAALNISNPKLKSTKEEP | |
| 103 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | N508D |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKEDAAYNAALNISNPKLKSTKEEP | |
| 104 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | Y511A |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAANAALNISNPKLKSTKEEP | |
| 105 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | A514G |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAGLNISNPKLKSTKEEP | |
| 106 | AKNTITKTLKLRIVRPYNSAEVEKIVADEKNNREKIALEKNKDKVKEACSKHLKVAAYCTTQV | Cas14/D326A/D510A/ |
| | ERNACLFCKARKLDDKFYQKLRGQFPDAVFWQEISEIFRQLQKQAAEIYNQSLIELYYEIFIK | IN516R |
| | GKGIANASSVEHYLSDVCYTRAAELFKNAAIASGLRSKIKSNFRLKELKNMKSGLPTTKSDNF | |
| | PIPLVKQKGGQYTGFEISNHNSDFIIKIPFGRWQVKKEIDKYRPWEKFDFEQVQKSPKPISLL | |
| | LSTQRRKRNKGWSKDEGTEAEIKKVMNGDYQTSYIEVKRGSKICEKSAWMLNLSIDVPKID | |
| | KGVDPSIIGGIAVGVKSPLVCAINNAFSRYSISDNDLFHFNKKMFARRRILLKKNRHKRAGH | |
| | GAKNKLKPITILTEKSERFRKKLIERWACEIADFFIKNKVGTVQMENLESMKRKEDSYFNIRL | |
| | RGFWPYAEMQNKIEFKLKQYGIEIRKVAPNNTSKTCSKCGHLNNYFNFEYRKKNKFPHFKC | |
| | EKCNFKENAAYNAALRISNPKLKSTKEEP | |
| 107 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | N519R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISRPKLKSTKEEP | |
| 108 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E527R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKREP | |
| 109 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | E528R |
| | FIKGKGIANASSVEHYLSDVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKERP | |
| 110 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 111 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151R |
| | FIKGKGIANASSVEHYLSRVCYTRAARLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 112 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/K154R |
| | FIKGKGIANASSVEHYLSRVCYTRAAELFRNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 113 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E241R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFRQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 114 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/K330R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 115 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E245N |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLNSMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 116 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E507R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKRNAAYNAALNISNPKLKSTKEEP | |
| 117 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/N516R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALRISNPKLKSTKEEP | |
| 118 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/N519R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISRPKLKSTKEEP | |
| 119 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E527R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKREP | |
| 120 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E528R |
| | FIKGKGIANASSVEHYLSRVCYTRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKERP | |
| 121 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E151R |
| | FIKGKGIANASSVEHYLSRVCYRRAARLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 122 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/K154R |
| | FIKGKGIANASSVEHYLSRVCYRRAAELFRNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 123 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E241R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFRQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 124 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/K330R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 125 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E425N |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLNSMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 126 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E507R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKRNAAYNAALNISNPKLKSTKEEP | |
| 127 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/N516R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALRISNPKLKSTKEEP | |
| 128 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/N519R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISRPKLKSTKEEP | |
| 129 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E527R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKREP | |
| 130 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E528R |
| | FIKGKGIANASSVEHYLSRVCYRRAAELFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKERP | |
| 131 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E151S |
| | FIKGKGIANASSVEHYLSRVCYRRAASLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 132 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E151G |
| | FIKGKGIANASSVEHYLSRVCYRRAAGLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 133 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/E151A |
| | FIKGKGIANASSVEHYLSRVCYRRAAALFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 134 | MAKNTITKTLKLRIVRPYNSAEVEKIVADEKNNREKIALEKNKDKVKEACSKHLKVAAYCTT | dCasMINI |
| | QVERNACLFCKARKLDDKFYQKLRGQFPDAVFWQEISEIFRQLQKQAAEIYNQSLIELYYEI | (Cas14/D326A/D510A/ |
| | FIKGKGIANASSVEHYLSRVCYTRAALLFKNAAIASGLRSKIKSNFRLKELKNMKSGLPTTKSD | D143R/E151L) |
| | NFPIPLVKQKGGQYTGFEISNHNSDFIIKIPFGRWQVKKEIDKYRPWEKFDFEQVQKSPKPI | |
| | SLLLSTQRRKRNKGWSKDEGTEAEIKKVMNGDYQTSYIEVKRGSKICEKSAWMLNLSIDVP | |
| | KIDKGVDPSIIGGIAVGVKSPLVCAINNAFSRYSISDNDLFHFNKKMFARRRILLKKNRHKRA | |
| | GHGAKNKLKPITILTEKSERFRKKLIERWACEIADFFIKNKVGTVQMENLESMKRKEDSYFNI | |
| | RLRGFWPYAEMQNKIEFKLKQYGIEIRKVAPNNTSKTCSKCGHLNNYFNFEYRKKNKFPHF | |
| | KCEKCNFKENAAYNAALNISNPKLKSTKEEP | |
| 135 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151S |
| | FIKGKGIANASSVEHYLSRVCYTRAASLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 136 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151T |
| | FIKGKGIANASSVEHYLSRVCYTRAATLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 137 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151W |
| | FIKGKGIANASSVEHYLSRVCYTRAAWLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 138 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151F |
| | FIKGKGIANASSVEHYLSRVCYTRAAFLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 139 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151C |
| | FIKGKGIANASSVEHYLSRVCYTRAACLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 140 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151K |
| | FIKGKGIANASSVEHYLSRVCYTRAAKLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 141 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151G |
| | FIKGKGIANASSVEHYLSRVCYTRAAGLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 142 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151I |
| | FIKGKGIANASSVEHYLSRVCYTRAAILFKNAAIASGLRSKIKSNFRLKELKNMKSGLPTTKSD | |
| | NFPIPLVKQKGGQYTGFEISNHNSDFIIKIPFGRWQVKKEIDKYRPWEKFDFEQVQKSPKPI | |
| | SLLLSTQRRKRNKGWSKDEGTEAEIKKVMNGDYQTSYIEVKRGSKICEKSAWMLNLSIDVP | |
| | KIDKGVDPSIIGGIAVGVKSPLVCAINNAFSRYSISDNDLFHFNKKMFARRRILLKKNRHKRA | |
| | GHGAKNKLKPITILTEKSERFRKKLIERWACEIADFFIKNKVGTVQMENLESMKRKEDSYFNI | |
| | RLRGFWPYAEMQNKIEFKLKQYGIEIRKVAPNNTSKTCSKCGHLNNYFNFEYRKKNKFPHF | |
| | KCEKCNFKENAAYNAALNISNPKLKSTKEEP | |
| 143 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151P |
| | FIKGKGIANASSVEHYLSRVCYTRAAPLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 144 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151H |
| | FIKGKGIANASSVEHYLSRVCYTRAAHLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 145 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151A |
| | FIKGKGIANASSVEHYLSRVCYTRAAALFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 146 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151V |
| | FIKGKGIANASSVEHYLSRVCYTRAAVLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 147 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151Q |
| | FIKGKGIANASSVEHYLSRVCYTRAAQLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 148 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151M |
| | FIKGKGIANASSVEHYLSRVCYTRAAMLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 149 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151D |
| | FIKGKGIANASSVEHYLSRVCYTRAADLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 150 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151N |
| | FIKGKGIANASSVEHYLSRVCYTRAANLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 151 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/E151Y |
| | FIKGKGIANASSVEHYLSRVCYRRAAYLFKNAAIASGLRSKIKSNFRLKELKNMKSGLPTTKS | |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVKSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKEEP | |
| 152 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/K330R/ |
| | FIKGKGIANASSVEHYLSRVCYRRAAALFKNAAIASGLRSKIKSNFRLKELKNMKSGLPTTKS | E151A/E527R |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKREP | |
| 153 | MAKNTITKTLKLRIVRPYNSAEVEKIVADEKNNREKIALEKNKDKVKEACSKHLKVAAYCTT | Cas14/D326A/D510A/ |
| | QVERNACLFCKARKLDDKFYQKLRGQFPDAVFWQEISEIFRQLQKQAAEIYNQSLIELYYEI | D143R/T147R/K330R/ |
| | FIKGKGIANASSVEHYLSRVCYRRAAALFKNAAIASGLRSKIKSNFRLKELKNMKSGLPTTKS | E151A/E528R |
| | DNFPIPLVKQKGGQYTGFEISNHNSDFIIKIPFGRWQVKKEIDKYRPWEKFDFEQVQKSPK | |
| | PISLLLSTQRRKRNKGWSKDEGTEAEIKKVMNGDYQTSYIEVKRGSKICEKSAWMLNLSID | |
| | VPKIDKGVDPSIIGGIAVGVRSPLVCAINNAFSRYSISDNDLFHFNKKMFARRRILLKKNRHK | |
| | RAGHGAKNKLKPITILTEKSERFRKKLIERWACEIADFFIKNKVGTVQMENLESMKRKEDSY | |
| | FNIRLRGFWPYAEMQNKIEFKLKQYGIEIRKVAPNNTSKTCSKCGHLNNYFNFEYRKKNKF | |
| | PHFKCEKCNFKENAAYNAALNISNPKLKSTKERP | |
| 154 | PKKKRKVGSGSSKLEKFTNCYSLSKTLRFKAIPVGKTQENIDNKRLLVEDEKRAEDYKGVKKL | pSLQ7347 |
| | LDRYYLSFINDVLHSIKLKNLNNYISLFRKKTRTEKENKELENLEINLRKEIAKAFKGNEGYKSL | pHR-PGK-SV40_NLS- |
| | FKKDIIETILPEFLDDKDEIALVNSFNGFTTAFTGFFDNRENMFSEEAKSTSIAFRCINENLTRY | dLbCpfl-KOX1_KRAB-c- |
| | ISNMDIFEKVDAIFDKHEVQEIKEKILNSDYDVEDFFEGEFFNFVLTQEGIDVYNAIIGGFVTE | Myc_NLS-P2A- |
| | SGEKIKGLNEYINLYNQKTKQKLPKFKPLYKQVLSDRESLSFYGEGYTSDEEVLEVFRNTLNK | mCherry-WPRE |
| | NSEIFSSIKKLEKLFKNFDEYSSAGIFVKNGPAISTISKDIFGEWNVIRDKWNAEYDDIHLKKK | SV40_NLS-dLbCpfl- |
| | AVVTEKYEDDRRKSFKKIGSFSLEQLQEYADADLSVVEKLKEIIIQKVDEIYKVYGSSEKLFDA | KOX1_KRAB-c- |
| | DFVLEKSLKKNDAVVAIMKDLLDSVKSFENYIKAFFGEGKETNRDESFYGDFVLAYDILLKVD | Myc_NLS-P2A-mCherry |
| | HIYDAIRNYVTQKPYSKDKFKLYFQNPQFMGGWDKDKETDYRATILRYGSKYYLAIMDKKY | |
| | AKCLQKIDKDDVNGNYEKINYKLLPGPNKMLPKVFFSKKWMAYYNPSEDIQKIYKNGTFKK | |
| | GDMFNLNDCHKLIDFFKDSISRYPKWSNAYDFNFSETEKYKDIAGFYREVEEQGYKVSFES | |
| | ASKKEVDKLVEEGKLYMFQIYNKDFSDKSHGTPNLHTMYFKLLFDENNHGQIRLSGGAELF | |
| | MRRASLKKEELVVHPANSPIANKNPDNPKKTTTLSYDVYKDKRFSEDQYELHIPIAINKCPK | |
| | NIFKINTEVRVLLKHDDNPYVIGIARGERNLLYIVVVDGKGNIVEQYSLNEIINNENGIRIKTD | |
| | YHSLLDKKEKERFEARQNWTSIENIKELKAGYISQVVHKICELVEKYDAVIALEDLNSGFKNS | |
| | RVKVEKQVYQKFEKMLIDKLNYMVDKKSNPCATGGALKGYQITNKFESFKSMSTQNGFIF | |
| | YIPAWLTSKIDPSTGFVNLLKTKYTSIADSKKFISSFDRIMYVPEEDLFEFALDYKNFSRTDAD | |
| | YIKKWKLYSYGNRIRIFRNPKKNNVFDWEEVCLTSAYKELFNKYGINYQQGDIRALLCEQSD | |
| | KAFYSSFMALMSLMLQMRNSITGRTDVDFLISPVKNSDGIFYDSRNYEAQENAILPKNAD | |
| | ANGAYNIARKVLWAIGQFKKAEDEKLDKVKIAISNKEWLEYAQTSVKHGSYPYDVPDYASL | |
| | GSGDGIGSGSNGSSLMDAKSLTAWSRTLVTFKDVFVDFTREEWKLLDTAQQIVYRNVML | |
| | ENYKNLVSLGYQLTKPDVILRLEKGEEPHMGGGSGEDPAAKRVKLDMGSGATNFSLLKQA | |
| | GDVEENPGPVSKGEEDNMAIIKEFMRFKVHMEGSVNGHEFEIEGEGEGRPYEGTQTAKL | |
| | KVTKGGPLPFAWDILSPQFMYGSKAYVKHPADIPDYLKLSFPEGFKWERVMNFEDGGVV | |
| | TVTQDSSLQDGEFIYKVKLRGTNFPSDGPVMQKKTMGWEASSERMYPEDGALKGEIKQR | |
| | LKLKDGGHYDAEVKTTYKAKKPVQLPGAYNVNIKLDITSHNEDYTIVEQYERAEGRHSTGG | |
| | MDELYK* | |
| 155 | PKKKRKVGSGSSKLEKFTNCYSLSKTLRFKAIPVGKTQENIDNKRLLVEDEKRAEDYKGVKKL | pSLQ7348 |
| | LDRYYLSFINDVLHSIKLKNLNNYISLFRKKTRTEKENKELENLEINLRKEIAKAFKGNEGYKSL | pHR-PGK-SV40_NLS- |
| | FKKDIIETILPEFLDDKDEIALVNSFNGFTTAFTGFFDNRENMFSEEAKSTSIAFRCINENLTRY | dLbCpfl-ZIM3_KRAB-c- |
| | ISNMDIFEKVDAIFDKHEVQEIKEKILNSDYDVEDFFEGEFFNFVLTQEGIDVYNAIIGGFVTE | Myc_NLS-P2A- |
| | SGEKIKGLNEYINLYNQKTKQKLPKFKPLYKQVLSDRESLSFYGEGYTSDEEVLEVFRNTLNK | mCherry-WPRE |
| | NSEIFSSIKKLEKLFKNFDEYSSAGIFVKNGPAISTISKDIFGEWNVIRDKWNAEYDDIHLKKK | SV40_NLS-dLbCpfl- |
| | AVVTEKYEDDRRKSFKKIGSFSLEQLQEYADADLSVVEKLKEIIIQKVDEIYKVYGSSEKLFDA | ZIM3_KRAB-c- |
| | DFVLEKSLKKNDAVVAIMKDLLDSVKSFENYIKAFFGEGKETNRDESFYGDFVLAYDILLKVD | Myc_NLS-P2A-mCherry |
| | HIYDAIRNYVTQKPYSKDKFKLYFQNPQFMGGWDKDKETDYRATILRYGSKYYLAIMDKKY | |
| | AKCLQKIDKDDVNGNYEKINYKLLPGPNKMLPKVFFSKKWMAYYNPSEDIQKIYKNGTFKK | |
| | GDMFNLNDCHKLIDFFKDSISRYPKWSNAYDFNFSETEKYKDIAGFYREVEEQGYKVSFES | |
| | ASKKEVDKLVEEGKLYMFQIYNKDFSDKSHGTPNLHTMYFKLLFDENNHGQIRLSGGAELF | |
| | MRRASLKKEELVVHPANSPIANKNPDNPKKTTTLSYDVYKDKRFSEDQYELHIPIAINKCPK | |
| | NIFKINTEVRVLLKHDDNPYVIGIARGERNLLYIVVVDGKGNIVEQYSLNEIINNFNGIRIKTD | |
| | YHSLLDKKEKERFEARQNWTSIENIKELKAGYISQVVHKICELVEKYDAVIALEDLNSGFKNS | |
| | RVKVEKQVYQKFEKMLIDKLNYMVDKKSNPCATGGALKGYQITNKFESFKSMSTQNGFIF | |
| | YIPAWLTSKIDPSTGFVNLLKTKYTSIADSKKFISSFDRIMYVPEEDLFEFALDYKNFSRTDAD | |
| | YIKKWKLYSYGNRIRIFRNPKKNNVFDWEEVCLTSAYKELFNKYGINYQQGDIRALLCEQSD | |
| | KAFYSSFMALMSLMLQMRNSITGRTDVDFLISPVKNSDGIFYDSRNYEAQENAILPKNAD | |
| | ANGAYNIARKVLWAIGQFKKAEDEKLDKVKIAISNKEWLEYAQTSVKHGSYPYDVPDYASL | |
| | GSGDGIGSGSNGSSLMNNSQGRVTFEDVTVNFTQGEWQRLNPEQRNLYRDVMLENYS | |
| | NLVSVGQGETTKPDVILRLEQGKEPWLEEEEVLGSGRAEKNGDIGGQIWKPKDVKESLAR | |
| | EVPSINKEHMGGGSGEDPAAKRVKLDMGSGATNFSLLKQAGDVEENPGPVSKGEEDNM | |
| | AIIKEFMRFKVHMEGSVNGHEFEIEGEGEGRPYEGTQTAKLKVTKGGPLPFAWDILSPQF | |
| | MYGSKAYVKHPADIPDYLKLSFPEGFKWERVMNFEDGGVVTVTQDSSLQDGEFIYKVKLR | |
| | GTNFPSDGPVMQKKTMGWEASSERMYPEDGALKGEIKQRLKLKDGGHYDAEVKTTYKA | |
| | KKPVQLPGAYNVNIKLDITSHNEDYTIVEQYERAEGRHSTGGMDELYK* | |
| 156 | PKKKRKVGSGSAKNTITKTLKLRIVRPYNSAEVEKIVADEKNNREKIALEKNKDKVKEACSKH | pSL9701 |
| | LKVAAYCTTQVERNACLFCKARKLDDKFYQKLRGQFPDAVFWQEISEIFRQLQKQAAEIYN | pHR-PGK-SV40_NLS- |
| | QSLIELYYEIFIKGKGIANASSVEHYLSDVCYTRAAELFKNAAIASGLRSKIKSNFRLKELKNMK | Cas14/D326A/D510A- |
| | SGLPTTKSDNFPIPLVKQKGGQYTGFEISNHNSDFIIKIPFGRWQVKKEIDKYRPWEKFDFE | KOX1_KRAB-c- |
| | QVQKSPKPISLLLSTQRRKRNKGWSKDEGTEAEIKKVMNGDYQTSYIEVKRGSKICEKSAW | Myc_NLS-mCherry- |
| | MLNLSIDVPKIDKGVDPSIIGGIAVGVKSPLVCAINNAFSRYSISDNDLFHFNKKMFARRRIL | WPRE |
| | LKKNRHKRAGHGAKNKLKPITILTEKSERFRKKLIERWACEIADFFIKNKVGTVQMENLESM | SV40_NLS- |
| | KRKEDSYFNIRLRGFWPYAEMQNKIEFKLKQYGIEIRKVAPNNTSKTCSKCGHLNNYFNFEY | Cas14/D326A/D510A- |
| | RKKNKFPHFKCEKCNFKENAAYNAALNISNPKLKSTKEEPAYPYDVPDYASLGSGDGIGSGS | KOX1_KRAB-c- |
| | NGSSLMDAKSLTAWSRTLVTFKDVFVDFTREEWKLLDTAQQIVYRNVMLENYKNLVSLGY | Myc_NLS-mCherry |
| | QLTKPDVILRLEKGEEPHMGGGSGEDPAAKRVKLDMGSGVSKGEEDNMAIIKEFMRFKV | |
| | HMEGSVNGHEFEIEGEGEGRPYEGTQTAKLKVTKGGPLPFAWDILSPQFMYGSKAYVKH | |
| | PADIPDYLKLSFPEGFKWERVMNFEDGGVVTVTQDSSLQDGEFIYKVKLRGTNFPSDGPV | |
| | MQKKTMGWEASSERMYPEDGALKGEIKQRLKLKDGGHYDAEVKTTYKAKKPVQLPGAY | |
| | NVNIKLDITSHNEDYTIVEQYERAEGRHSTGGMDELYK* | |
| 157 | PKKKRKVGSGSAKNTITKTLKLRIVRPYNSAEVEKIVADEKNNREKIALEKNKDKVKEACSKH | pSL9702 |
| | LKVAAYCTTQVERNACLFCKARKLDDKFYQKLRGQFPDAVFWQEISEIFRQLQKQAAEIYN | pHR-PGK-SV40_NLS- |
| | QSLIELYYEIFIKGKGIANASSVEHYLSDVCYTRAAELFKNAAIASGLRSKIKSNFRLKELKNMK | Cas14/D326A/D510A- |
| | SGLPTTKSDNFPIPLVKQKGGQYTGFEISNHNSDFIIKIPFGRWQVKKEIDKYRPWEKFDFE | ZIM3_KRAB-c- |
| | QVQKSPKPISLLLSTQRRKRNKGWSKDEGTEAEIKKVMNGDYQTSYIEVKRGSKICEKSAW | Myc_NLS-mCherry- |
| | MLNLSIDVPKIDKGVDPSIIGGIAVGVKSPLVCAINNAFSRYSISDNDLFHFNKKMFARRRIL | WPRE |
| | LKKNRHKRAGHGAKNKLKPITILTEKSERFRKKLIERWACEIADFFIKNKVGTVQMENLESM | SV40_NLS- |
| | KRKEDSYFNIRLRGFWPYAEMQNKIEFKLKQYGIEIRKVAPNNTSKTCSKCGHLNNYENFEY | Cas14/D326A/D510A- |
| | RKKNKFPHFKCEKCNFKENAAYNAALNISNPKLKSTKEEPAYPYDVPDYASLGSGDGIGSGS | ZIM3_KRAB-c- |
| | NGSSLMNNSQGRVTFEDVTVNFTQGEWQRLNPEQRNLYRDVMLENYSNLVSVGQGET | Myc_NLS-mCherry |
| | TKPDVILRLEQGKEPWLEEEEVLGSGRAEKNGDIGGQIWKPKDVKESLAREVPSINKEHM | |
| | GGGSGEDPAAKRVKLDMGSGVSKGEEDNMAIIKEFMRFKVHMEGSVNGHEFEIEGEGE | |
| | GRPYEGTQTAKLKVTKGGPLPFAWDILSPQFMYGSKAYVKHPADIPDYLKLSFPEGFKWE | |
| | RVMNFEDGGVVTVTQDSSLQDGEFIYKVKLRGTNFPSDGPVMQKKTMGWEASSERMY | |
| | PEDGALKGEIKQRLKLKDGGHYDAEVKTTYKAKKPVQLPGAYNVNIKLDITSHNEDYTIVEQ | |
| | YERAEGRHSTGGMDELYK* | |
| 158 | PKKKRKVGSGSAKNTITKTLKLRIVRPYNSAEVEKIVADEKNNREKIALEKNKDKVKEACSKH | pSL9703 |
| | LKVAAYCTTQVERNACLFCKARKLDDKFYQKLRGQFPDAVFWQEISEIFRQLQKQAAEIYN | pHR-PGK-SV40_NLS- |
| | QSLIELYYEIFIKGKGIANASSVEHYLSRVCYTRAAELFKNAAIASGLRSKIKSNFRLKELKNMK | Cas14/D143R/D326A/ |
| | SGLPTTKSDNFPIPLVKQKGGQYTGFEISNHNSDFIIKIPFGRWQVKKEIDKYRPWEKFDFE | D510A-KOX1_KRAB-c- |
| | QVQKSPKPISLLLSTQRRKRNKGWSKDEGTEAEIKKVMNGDYQTSYIEVKRGSKICEKSAW | Myc_NLS-mCherry- |
| | MLNLSIDVPKIDKGVDPSIIGGIAVGVKSPLVCAINNAFSRYSISDNDLFHFNKKMFARRRIL | WPRE |
| | LKKNRHKRAGHGAKNKLKPITILTEKSERFRKKLIERWACEIADFFIKNKVGTVQMENLESM | SV40_NLS- |
| | KRKEDSYFNIRLRGFWPYAEMQNKIEFKLKQYGIEIRKVAPNNTSKTCSKCGHLNNYFNFEY | Cas14/D143R/D326A/ |
| | RKKNKFPHFKCEKCNFKENAAYNAALNISNPKLKSTKEEPAYPYDVPDYASLGSGDGIGSGS | D510A-KOX1_KRAB-c- |
| | NGSSLMDAKSLTAWSRTLVTFKDVFVDFTREEWKLLDTAQQIVYRNVMLENYKNLVSLGY | Myc_NLS-mCherry |
| | QLTKPDVILRLEKGEEPHMGGGSGEDPAAKRVKLDMGSGVSKGEEDNMAIIKEFMRFKV | |
| | HMEGSVNGHEFEIEGEGEGRPYEGTQTAKLKVTKGGPLPFAWDILSPQFMYGSKAYVKH | |
| | PADIPDYLKLSFPEGFKWERVMNFEDGGVVTVTQDSSLQDGEFIYKVKLRGTNFPSDGPV | |
| | MQKKTMGWEASSERMYPEDGALKGEIKQRLKLKDGGHYDAEVKTTYKAKKPVQLPGAY | |
| | NVNIKLDITSHNEDYTIVEQYERAEGRHSTGGMDELYK* | |
| 159 | PKKKRKVGSGSAKNTITKTLKLRIVRPYNSAEVEKIVADEKNNREKIALEKNKDKVKEACSKH | pSL9704 |
| | LKVAAYCTTQVERNACLFCKARKLDDKFYQKLRGQFPDAVFWQEISEIFRQLQKQAAEIYN | pHR-PGK-SV40_NLS- |
| | QSLIELYYEIFIKGKGIANASSVEHYLSRVCYTRAAELFKNAAIASGLRSKIKSNFRLKELKNMK | Cas14a1/D143R/D326A/ |
| | SGLPTTKSDNFPIPLVKQKGGQYTGFEISNHNSDFIIKIPFGRWQVKKEIDKYRPWEKFDFE | D510A-ZIM3_KRAB-c- |
| | QVQKSPKPISLLLSTQRRKRNKGWSKDEGTEAEIKKVMNGDYQTSYIEVKRGSKICEKSAW | Myc_NLS-mCherry- |
| | MLNLSIDVPKIDKGVDPSIIGGIAVGVKSPLVCAINNAFSRYSISDNDLFHFNKKMFARRRIL | WPRE |
| | LKKNRHKRAGHGAKNKLKPITILTEKSERFRKKLIERWACEIADFFIKNKVGTVQMENLESM | SV40_NLS- |
| | KRKEDSYFNIRLRGFWPYAEMQNKIEFKLKQYGIEIRKVAPNNTSKTCSKCGHLNNYENFEY | Cas14a1/D143R/D326A/ |
| | RKKNKFPHFKCEKCNFKENAAYNAALNISNPKLKSTKEEPAYPYDVPDYASLGSGDGIGSGS | D510A-ZIM3_KRAB-c- |
| | NGSSLMNNSQGRVTFEDVTVNFTQGEWQRLNPEQRNLYRDVMLENYSNLVSVGQGET | Myc_NLS-mCherry |
| | TKPDVILRLEQGKEPWLEEEEVLGSGRAEKNGDIGGQIWKPKDVKESLAREVPSINKEHM | |
| | GGGSGEDPAAKRVKLDMGSGVSKGEEDNMAIIKEFMRFKVHMEGSVNGHEFEIEGEGE | |
| | GRPYEGTQTAKLKVTKGGPLPFAWDILSPQFMYGSKAYVKHPADIPDYLKLSFPEGFKWE | |
| | RVMNFEDGGVVTVTQDSSLQDGEFIYKVKLRGTNFPSDGPVMQKKTMGWEASSERMY | |
| | PEDGALKGEIKQRLKLKDGGHYDAEVKTTYKAKKPVQLPGAYNVNIKLDITSHNEDYTIVEQ | |
| | YERAEGRHSTGGMDELYK* | |
| 160 | PKKKRKVGSGSAKNTITKTLKLRIVRPYNSAEVEKIVADEKNNREKIALEKNKDKVKEACSKH | pSL9705 |
| | LKVAAYCTTQVERNACLFCKARKLDDKFYQKLRGQFPDAVFWQEISEIFRQLQKQAAEIYN | pHR-PGK-SV40_NLS- |
| | QSLIELYYEIFIKGKGIANASSVEHYLSRVCYRRAAELFKNAAIASGLRSKIKSNFRLKELKNMK | Cas14a1/D143R/T147R/ |
| | SGLPTTKSDNFPIPLVKQKGGQYTGFEISNHNSDFIIKIPFGRWQVKKEIDKYRPWEKFDFE | D326A/D510A- |
| | QVQKSPKPISLLLSTQRRKRNKGWSKDEGTEAEIKKVMNGDYQTSYIEVKRGSKICEKSAW | KOX1_ |
| | MLNLSIDVPKIDKGVDPSIIGGIAVGVKSPLVCAINNAFSRYSISDNDLFHFNKKMFARRRIL | KRAB-c- |
| | LKKNRHKRAGHGAKNKLKPITILTEKSERFRKKLIERWACEIADFFIKNKVGTVQMENLESM | Myc_NLS-mCherry- |
| | KRKEDSYFNIRLRGFWPYAEMQNKIEFKLKQYGIEIRKVAPNNTSKTCSKCGHLNNYFNFEY | WPRE |
| | RKKNKFPHFKCEKCNFKENAAYNAALNISNPKLKSTKEEPAYPYDVPDYASLGSGDGIGSGS | SV40_NLS- |
| | NGSSLMDAKSLTAWSRTLVTFKDVFVDFTREEWKLLDTAQQIVYRNVMLENYKNLVSLGY | Cas14a1/D143R/T147R/ |
| | QLTKPDVILRLEKGEEPHMGGGSGEDPAAKRVKLDMGSGVSKGEEDNMAIIKEFMRFKV | D326A/D510A- |
| | HMEGSVNGHEFEIEGEGEGRPYEGTQTAKLKVTKGGPLPFAWDILSPQFMYGSKAYVKH | KOX1_KRAB-c- |
| | PADIPDYLKLSFPEGFKWERVMNFEDGGVVTVTQDSSLQDGEFIYKVKLRGTNFPSDGPV | Myc_NLS-mCherry |
| | MQKKTMGWEASSERMYPEDGALKGEIKQRLKLKDGGHYDAEVKTTYKAKKPVQLPGAY | |
| | NVNIKLDITSHNEDYTIVEQYERAEGRHSTGGMDELYK* | |
| 161 | PKKKRKVGSGSAKNTITKTLKLRIVRPYNSAEVEKIVADEKNNREKIALEKNKDKVKEACSKH | pSL9706 |
| | LKVAAYCTTQVERNACLFCKARKLDDKFYQKLRGQFPDAVFWQEISEIFRQLQKQAAEIYN | pHR-PGK-SV40_NLS- |
| | QSLIELYYEIFIKGKGIANASSVEHYLSRVCYRRAAELFKNAAIASGLRSKIKSNFRLKELKNMK | Cas14a1/D143R/T147R/ |
| | SGLPTTKSDNFPIPLVKQKGGQYTGFEISNHNSDFIIKIPFGRWQVKKEIDKYRPWEKFDFE | D326A/D510A- |
| | QVQKSPKPISLLLSTQRRKRNKGWSKDEGTEAEIKKVMNGDYQTSYIEVKRGSKICEKSAW | ZIM3_KRAB-c- |
| | MLNLSIDVPKIDKGVDPSIIGGIAVGVKSPLVCAINNAFSRYSISDNDLFHFNKKMFARRRIL | Myc_NLS-mCherry- |
| | LKKNRHKRAGHGAKNKLKPITILTEKSERFRKKLIERWACEIADFFIKNKVGTVQMENLESM | WPRE |
| | KRKEDSYFNIRLRGFWPYAEMQNKIEFKLKQYGIEIRKVAPNNTSKTCSKCGHLNNYFNFEY | SV40_NLS- |
| | RKKNKFPHFKCEKCNFKENAAYNAALNISNPKLKSTKEEPAYPYDVPDYASLGSGDGIGSGS | Cas14a1/D143R/T147R/ |
| | NGSSLMNNSQGRVTFEDVTVNFTQGEWQRLNPEQRNLYRDVMLENYSNLVSVGQGET | D326A/D510A- |
| | TKPDVILRLEQGKEPWLEEEEVLGSGRAEKNGDIGGQIWKPKDVKESLAREVPSINKEHM | ZIM3_KRAB-c- |
| | GGGSGEDPAAKRVKLDMGSGVSKGEEDNMAIIKEFMRFKVHMEGSVNGHEFEIEGEGE | Myc_NLS-mCherry |
| | GRPYEGTQTAKLKVTKGGPLPFAWDILSPQFMYGSKAYVKHPADIPDYLKLSFPEGFKWE | |
| | RVMNFEDGGVVTVTQDSSLQDGEFIYKVKLRGTNFPSDGPVMQKKTMGWEASSERMY | |
| | PEDGALKGEIKQRLKLKDGGHYDAEVKTTYKAKKPVQLPGAYNVNIKLDITSHNEDYTIVEQ | |
| | YERAEGRHSTGGMDELYK* | |

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A single-guide RNA (sgRNA) comprising an engineered CRISPR RNA (crRNA)/trans-activating CRISPR RNA (tracrRNA) fusion nucleotide sequence that is at least 80% identical to a wild-type crRNA/tracrRNA fusion nucleotide sequence, wherein:
the wild-type crRNA/tracrRNA fusion nucleotide sequence comprises (1) a 3′ region corresponding to an RNA stem-loop hairpin structure, (2) a poly-U region proximate to the 3′ region, and (3) a 5′ poly-G region; and
the engineered crRNA/tracrRNA fusion nucleotide sequence comprises one or more modifications to the wild-type crRNA/tracrRNA fusion nucleotide sequence, the modifications selected from the group consisting of:
substitution of at least one U of the poly-U region,
deletion of at least a portion of the 3′ region; and
deletion of at least a portion of the 5′ poly-G region.

2. The sgRNA of claim 1, wherein the wild-type crRNA/tracrRNA fusion nucleotide sequence is the sequence of SEQ ID NO: 7.

3. The sgRNA of claim 1, wherein the engineered crRNA/tracrRNA fusion nucleotide sequence comprises substitutions of at least one U of the poly-U region with a G.

4. The sgRNA of claim 3, wherein the engineered crRNA/tracrRNA fusion nucleotide sequence comprises the sequence of SEQ ID NO: 8.

5. The sgRNA of claim 3, wherein the engineered crRNA/tracrRNA fusion nucleotide sequence comprises deletion of at least a portion of the 3′ region.

6. The sgRNA of claim 5, wherein the engineered crRNA/tracrRNA fusion nucleotide sequence comprises the sequence of SEQ ID NO: 9.

7. The sgRNA of claim 5, wherein the engineered crRNA/tracrRNA fusion nucleotide sequence comprises deletion of at least a portion of the 5′ poly-G region.

8. The sgRNA of claim 7, wherein the engineered crRNA/tracrRNA fusion nucleotide sequence comprises the sequence of SEQ ID NO: 10.

9. The sgRNA of claim 1, further comprising a spacer nucleotide sequence corresponding to a 5′ Protospacer Adjacent Motif (PAM) having a 5′-TTTR-3′ nucleotide sequence.

10. A nucleic acid encoding the sgRNA of claim 1.

11. A vector comprising the nucleic acid of claim 10.

12. A system comprising:
a Cas protein; and
the sgRNA of claim 1.

13. The system of claim 12, wherein the Cas protein is an engineered Cas protein that is capable of modulating a target nucleic acid in eukaryotic cells, the Cas protein comprising an amino acid sequence of a native amino acid sequence of SEQ ID NO: 1 with one or more substitutions thereto, wherein the amino acid sequence is at least 95% identical to SEQ ID NO: 1, and wherein the one or more substitutions comprises at least one substitution at a position selected from the group consisting of: D143, K11, K73, T147, E151, K154, E241, D318, K330, K457, E425, E462, N504, E507, N516, N519, E527, and E528.

14. The system of claim 13, wherein the Cas protein has the amino acid sequence of SEQ ID NO: 5.

15. A system comprising:
a nucleic acid sequence encoding a Cas protein; and
the nucleic acid sequence of claim 10.

16. The system of claim 15, wherein the Cas protein is an engineered Cas protein that is capable of modulating a target nucleic acid in eukaryotic cells, the Cas protein comprising an amino acid sequence of a native amino acid sequence of SEQ ID NO: 1 with one or more substitutions thereto, wherein the amino acid sequence is at least 95% identical to SEQ ID NO: 1, and wherein the one or more substitutions comprises at least one substitution at a position selected from the group consisting of: D143, K11, K73, T147, E151, K154, E241, D318, K330, K457, E425, E462, N504, E507, N516, N519, E527, and E528.

17. A method of modulating one or more target nucleic acids in a cell, the method comprising contacting the cell with the sgRNA of claim 1.

18. A pharmaceutical composition comprising the sgRNA of claim 1.

19. A method of preventing or treating a genetic disorder in a subject, the method comprising administering to the subject an amount of the pharmaceutical composition of claim 18, wherein the amount is sufficient to modulate one or more target nucleic acids associated with the genetic disorder.

20. A method of treating an infection in a subject, the method comprising administering to the subject an amount of the pharmaceutical composition of claim 18, wherein the amount is sufficient to modulate one or more target nucleic acids associated with the infection.
